SEQUENCE LISTING (5 sequences)

SEQ ID NO: 1 - protein, 568 aa, synthetic construct
MVSKGEELFTGVVPILVELDGDVNGHKFSVSGEGEGDATYGKLTLKFICTTGKLPVPWPTLVTTLSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKLQGSFMKKNQNTEIGSFEMASKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGAALAFHYSYEHQDKIKAIVHAESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 2 - protein, 873 aa, synthetic construct
MRGSHHHHHHGMASMTGGQQMGRDLYDDDDKDRWGSEFMVSKGEELFTGVVPILVELDGDVNGHKFSVSGEGEGDATYGKLTLKFICTTGKLPVPWPTLVTTLSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKLQMQILWKKYVKENFEMNVDECGIEQGIPGLGYNYEVLKNAVIHYVTKGYGTFKFNGKVYNLKQGDIFILLKGMQVEYVASIDDPWEYYWIGFSGSNANEYLNRTSITNSCVANCEENSKIPQIILNMCEISKTYNPSRSDDILLLKELYSLLYALIEEFPKPFEYKDKELHTYIQDALNFINSNYMHSITVQEIADYVNLSRSYLYKMFIKNLGISPQRYLINLRMYKATLLLKSTKLPIGEVASSVGYSDSLLFSKTFSKHFSMSPLNYRNNQVNKPSIFELQMASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 3 - protein, 873 aa, synthetic construct
MRGSHHHHHHGMASMTGGQQMGRDLYDDDDKDRWGSEFMVSKGEELFTGVVPILVELDGDVNGHKFSVSGEGEGDATYGKLTLKFICTTGKLPVPWPTLVTTLSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKLQMQILWKKYVKENFEMNVDECGIEQGIPGLGYNYEVLKNAVIHYVTKGYGTFKFNGKVYNLKQGDIFILLKGMQVEYVASIDDPWEYYWIGFSGSNANEYLNRTSITNSCVANCEENSKIPQIILNMCEISKTYNPSRSDAALLLKELYSLLYALIEEFPKPFEYKDKELHTYIQDALNFINSNYMHSITVQEIADYVNLSRSYLYKMFIKNLGISPQRYLINLRMYKATLLLKSTKLPIGEVASSVGYSDSLLFSKTFSKHFSMSPLNYRNNQVNKPSIFELQMASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 4 - protein, 873 aa, synthetic construct
MRGSHHHHHHGMASMTGGQQMGRDLYDDDDKDRWGSEFMVSKGEELFTGVVPILVELDGDVNGHKFSVSGEGEGDATYGKLTLKFICTTGKLPVPWPTLVTTLSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKLQMQILWKKYVKESFEMNVDECGIEQGIPGLGYNYEVLKNAVIHYVTKGYGTFKFNGKVYNLKQGDIFILLKGMQVEYVASIDDPWEYYWIGFSGSNANEYLNRTSITNSCVANCEENSKIPQIILNMCEISKTYNPSRSDSALLLKELYSLLYALIEEFPKPFEYKDKELHTYIQDALNFINSNYMHSITVQEIADYVNLSRSYLYKMFIKNLGISPQRYLINLRMYKATLLLKSTKLPIGEVASSVGYSDSLLFSKTFSKHFSMSPLNYRNNQVNKPSIFELQMASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 5 - protein, 873 aa, synthetic construct
MRGSHHHHHHGMASMTGGQQMGRDLYDDDDKDRWGSEFMVSKGEELFTGVVPILVELDGDVNGHKFSVSGEGEGDATYGKLTLKFICTTGKLPVPWPTLVTTLSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKLQMQILWKKYVKENFEMNVDECGIEQGIPGLGYNYEVLKNAVIHYVTKGYGTFKFNGKVYNLKQGDIFILLKGMQVEYVASIDDPWEYYWIGFSGSNANEYLNRTSITNSCVANCEENSKIPQIILNMCEISKTYNPSRSDSAALAKELYSLLYALIEEFPKPFEYKDKELHTYIQDALNFINSNYMHSITVQEIADYVNLSRSYLYKMFIKNLGISPQRYLINLRMYKATLLLKSTKLPIGEVASSVGYSDSLLFSKTFSKHFSMSPLNYRNNQVNKPSIFELQMASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

(19) United States
(12) Patent Application Publication
(10) Pub. No.: US 2026/0337658 A1
(43) Pub. Date: Oct. 29, 2026

(54) METHOD OF DETECTING AN ANALYTE IN A SAMPLE

(71) Applicant: PPB TECHNOLOGY PTY LTD, Australian Capital Territory (AU)

(72) Inventors: Stephen TROWELL, Canberra (AU); Felix WEIHS, Canberra (AU)

(21) Appl. No.: 19/122,874

(22) PCT Filed: Oct. 20, 2023
(86) PCT No.: PCT/AU2023/051047

§ 371 Date: Apr. 21, 2025

(30) Foreign Application Priority Data
Oct. 21, 2022 (AU) 2022903116

(51) Int. Cl. C12Q 1/44; C12Q 1/37; G01N 21/76; G01N 33/04

(52) U.S. Cl. CPC C12Q 1/44; C12Q 1/37; G01N 21/763; G01N 33/04

(57) ABSTRACT

The invention provides a method of detecting an extracellular analyte in a sample. In some examples, there is provided a method of detecting an extracellular analyte in a sample, comprising: providing a biosensor system selected from (i) an extracellular bioluminescent sensor molecule comprising a donor lumiphore, an analyte-sensitive domain and an acceptor fluorophore; and (ii) a precursor system thereof, the precursor system comprising a pair of molecules adapted to react in solution to form the extracellular bioluminescent sensor molecule; incubating an assay composition comprising the sample and the biosensor system for an incubation period sufficient for the extracellular analyte, if present in the sample, to interact with the analyte-sensitive domain; and detecting light emitted from the assay composition after the incubation period, wherein the ratio of luminosity associated with the acceptor fluorophore to luminosity associated with the donor lumiphore indicates an interaction of the extracellular analyte with the analyte-sensitive domain, wherein at least one caged substrate compound is uncaged in the assay composition during the incubation period to produce an uncaged substrate compound which energises the donor lumiphore for the detecting.

### TECHNICAL FIELD

The invention relates to a method of detecting an extracellular analyte in a sample. The invention further relates to a system for detecting an extracellular analyte in a sample, to a BRET reagent system, to a solid biosensor composition, to a substrate dispensing system and to a substrate composition. While not so limited, the invention is of particular interest in on-the-spot diagnostic (bio) chemical analyses to be used in production or other non-laboratory environments, for example in analyses of food and beverage compositions.

### BACKGROUND OF INVENTION

There is a need for on-the-spot diagnostic biochemical testing in many fields such as human health care, environmental monitoring, managing agricultural production, and to support the manufacture of food, pharmaceutical and personal care products. In the food industry, as one example, on-the-spot tests can in principle provide the most rapid information on (bio) chemical composition of food ingredients and finished products for quality control and cost efficiencies throughout the value chain. The desired goal is for on-the-spot testing methods to deliver accurate and precise results quickly, even when performed by minimally-trained staff in production settings.

Chemiluminescence based techniques, such as bioluminescence Resonance Energy Transfer (BRET), are attractive for biochemical testing, potentially allowing highly sensitive real-time optical detection of a wide range of analytes in many different types of samples. Because the detected light is generated internally by the biosensor, chemiluminescence analyses can be used on complex fluid samples containing auto-fluorescent molecules which confound analyses relying on an external light source. BRET analyses often use BRET bioluminescent sensor molecules which include a donor lumiphore and an acceptor fluorophore connected by a linker which includes an analyte-sensitive domain. The donor lumiphore and an acceptor fluorophore are held in sufficiently close proximity (typically <10 nm separation) and alignment to allow Forster resonance energy transfer from the donor lumiphore to the acceptor fluorophore. When the donor lumiphore (e.g. a luciferase such as Renilla luciferase; RLuc), acts on a suitable substrate (e.g. a luciferin such as a coelenterazine molecule), light is thus emitted from both the donor lumiphore and the acceptor fluorophore, with the relative intensities (luminosities) being highly dependent on the separation distance and relative orientations of the two moieties. Therefore, an interaction of an analyte with the analyte-sensitive domain, for example by binding or cleavage, can be detected as a change in the ratio of luminosity associated with the acceptor fluorophore to luminosity associated with the donor lumiphore (the BRET ratio).

BRET biosensor systems containing various donor lumiphores, acceptor fluorophores, analyte-sensitive domains and substrates have been reported. For example, the BRET² platform, which uses RLuc as donor lumiphore, green fluorescent protein (GFP²) as acceptor fluorophore and bisdeoxycoelenterazine (coelenterazine 400a) as substrate, has been used in biosensing techniques for detecting proteases (e.g. plasmin in milk; Australian patent 2015243093) or sugars (e.g. maltose or lactose; Australian patent 2018315053).

One difficulty with the use of BRET technology is the instability of the reagents, including the oxidation-susceptible substrate, which can affect the accuracy of the test results. Another issue is the difficulty in matching the kinetics of the bioluminescence reaction, i.e. enzymatic oxidation of the substrate by the donor lumiphore, with the kinetics of the analyte interaction with the bioluminescent sensor molecule, e.g. via binding to or cleavage of the analyte-sensitive domain. The decay in luminosity as the substrate is consumed can affect the accuracy of analysis since the BRET ratio is typically not entirely independent of the total luminosity, particularly for analyses requiring longer incubation times for the analyte to interact with the bioluminescent sensor molecule.

These issues can be addressed in a laboratory environment via ultracold storage of reagents, complex and rigorous assay protocols, or the use of microfluidic techniques, but such measures are impractical for widespread use in many on-the-spot test applications where less sophisticated infrastructure and operators are available. More fundamentally, the robustness of BRET assays can be improved to a degree by optimising the bioluminescent sensor molecule and corresponding substrate for enhanced stability and persistence of luminescence, but this can compromise the sensitivity of the sensor and thus its usefulness for certain analyte detections.

It would be desirable to develop chemiluminescent-based analysis technologies which utilise room temperature-stable reagents and which minimise the complexity of each assay without unacceptably compromising the reliability and sensitivity of the analysis.

There is therefore an ongoing need for methods and systems for detecting an analyte in a sample, which at least partially address one or more of the above-mentioned short-comings, or provide a useful alternative.

A reference herein to a patent document or other matter which is given as prior art is not to be taken as an admission that the document or matter was known or that the information it contains was part of the common general knowledge as at the priority date of any of the claims.

### SUMMARY OF INVENTION

In accordance with a first aspect the invention provides a method of detecting an extracellular analyte in a sample, comprising: providing a biosensor system selected from (i) an extracellular bioluminescent sensor molecule comprising a donor lumiphore, an analyte-sensitive domain and an acceptor fluorophore; and (ii) a precursor system thereof, the precursor system comprising a pair of molecules adapted to react in solution to form the extracellular bioluminescent sensor molecule; incubating an assay composition comprising the sample and the biosensor system for an incubation period sufficient for the extracellular analyte, if present in the sample, to interact with the analyte-sensitive domain; and detecting light emitted from the assay composition after the incubation period, wherein the ratio of luminosity associated with the acceptor fluorophore to luminosity associated with the donor lumiphore indicates an interaction of the extracellular analyte with the analyte-sensitive domain, wherein at least one caged substrate compound is uncaged in the assay composition during the incubation period to produce an uncaged substrate compound which energises the donor lumiphore for the detecting.

The addition of one or more caged (i.e. protected) substrate compounds to the assay composition, configured to uncage (i.e. deprotect) in situ to a substrate of the (BRET) bioluminescent sensor molecule, has been found to provide significant and surprising benefits in the methods disclosed herein, making them particularly suitable for on-the-spot diagnostic tests in production and other non-laboratory environments. Uncaging of the substrate compound(s) in the assay allows the luminescence generated by the bioluminescent sensor molecule to be maximised and sustained over an extended detection window which comfortably spans the detection time at the end of the incubation period. This ensures that the accuracy of the analysis is not compromised by luminescence decay and is insensitive to operator-specific variability when implementing the test. The luminescence profile can be optimised for analyte detection after short (e.g. 1-5 minutes) or long (e.g. 10-30+ minute) incubation times, depending on the nature of the analyte-biosensor interaction, by selection of suitable caged substrates and uncaging agents. In some embodiments, two or more caged substrate compounds, having different uncaging kinetics, can be used in combination to further extend and tailor the detection window.

Furthermore, it has surprisingly been found by experiment that solutions of caged substrate compounds can be stored at room temperature for prolonged periods, in excess of a year for certain compounds, without compromising the performance in subsequent BRET analyses. This advantageously allows the substrate to be stored in solution for dispensing when required, thus minimising the number of steps involved in the test methodology while avoiding errors caused by reagent degradation.

As a result of these advantages, the method of the present invention may be implemented in a simple and robust workflow in a production or other non-laboratory environment.

In some embodiments, the at least one caged substrate compound is present in the assay composition from the beginning of the incubation period.

In some embodiments, the at least one caged substrate compound is uncaged by an uncaging agent present in the assay composition. The uncaging agent may be present in the assay composition from the beginning of the incubation period.

In some embodiments, the uncaging agent is extraneous to the sample. The uncaging agent may be an esterase which is extraneous to the sample.

In some embodiments, the uncaging agent is endogenous to the sample. The uncaging agent may be an esterase which is endogenous to the sample.

In some embodiments, the incubation period is between 1 minute and 30 minutes. In some embodiments, the incubation period is between 5 and 15 minutes.

In some embodiments, the at least one caged substrate compound is uncaged to provide a luminosity associated with the acceptor fluorophore, in the absence of the extracellular analyte, greater than 50% of maximum when detecting the light, or greater than 80% of maximum when detecting the light. The luminosity associated with the acceptor fluorophore, in the absence of the extracellular analyte, may be greater than 80% of maximum throughout a detection window of at least 3 minutes, or at least 5 minutes, wherein the light is detected during the detection window.

In some embodiments, at least two different caged substrate compounds present in the assay composition from the beginning of the incubation period are uncaged during the incubation period, thereby producing the uncaged substrate compound at different reaction rates.

In some embodiments, each caged substrate compound is a caged coelenterazine compound comprising a carbonyl protecting group at the 3′-position.

In some embodiments, each caged substrate compound has a structure according to Formula 1 or Formula 2:

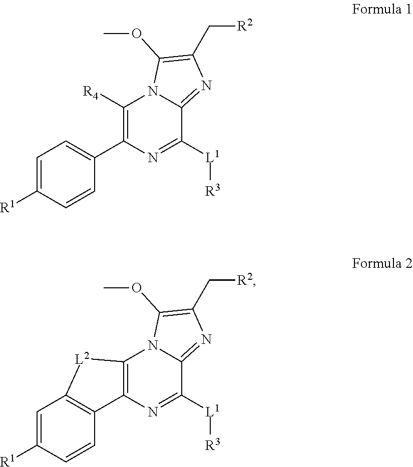

wherein: each R¹ is independently selected from H, OH, OR^{alk} where R^{alk} is C₁-C₆ alkyl, and NH₂, each R² is independently selected from H, Ar, and (CH₂)ₙAr, where each Ar is independently a carbocyclic aromatic group optionally substituted with OH, C₁-C₆ alkyl or halogen, and n is 1 or 2, each L¹ is independently selected from CH₂ or S, each R³ is independently selected from C₆H₅, CH₂C₆H₅, C₁-C₆ alkyl, and C₁-C₆ cycloalkyl, R⁴ is independently selected from H and CH₃, L² is independently selected from (CH₂)ₙ where n is 1-3 and CH═CH, and each X is independently a carbonyl protecting group.

In some embodiments, each X has a structure according to Formula 3, Formula 4 or Formula 5:

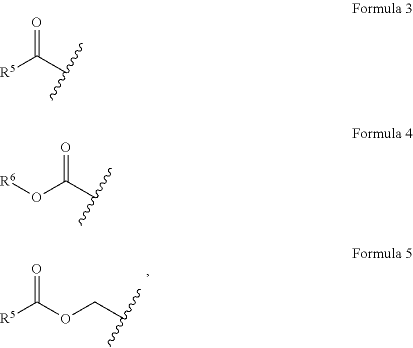

wherein R⁵, R⁶ and R⁷ are each independently optionally substituted C₁-C₁₂ alkyl, C₁-C₁₂ cycloalkyl or C₁-C₁₂ aryl.

In some embodiments, the uncaged substrate compound is a coelenterazine compound. In some embodiments, coelenterazine compound is bisdeoxycoelenterazine.

In some embodiments, the at least one caged substrate compound is introduced to the assay composition in a room temperature-stable substrate solution, optionally by dispensing the substrate solution drop-wise into the assay composition. The substrate solution may be a non-aqueous solution comprising a water-miscible aprotic solvent, for example DMSO. The method may further comprise storing the substrate solution for at least one week, or at least one month, or at least six months, before introducing the substrate solution to the assay composition.

In some embodiments, the method comprises preparing the assay composition by a method comprising dispersing a solid composition comprising the biosensor system in a liquid. The solid composition may be a freeze-dried solid composition. The liquid may comprise, or consist of, the sample. The solid composition may further comprise an uncaging agent for uncaging the at least one caged substrate compound in the assay composition. The assay composition may be prepared and incubated in an assay vessel in which the solid composition was previously freeze-dried.

In some embodiments, the assay composition is prepared and incubated in an assay vessel and the light emitted from the assay composition is detected through the walls of the assay vessel.

In some embodiments, the extracellular bioluminescent sensor molecule is a BRET² biosensor.

In some embodiments, the extracellular analyte is selected from the group consisting of an enzyme and a carbohydrate. In some embodiments, the extracellular analyte is a protease.

In some embodiments, the sample is a food or beverage composition, for example a dairy composition.

In some embodiments, the method comprises:
providing the sample, as a liquid, for analysis;
providing an assay vessel containing a solid composition, the solid composition comprising the extracellular bioluminescent sensor molecule and optionally an uncaging agent;
providing a substrate storage container containing a room temperature-storable substrate solution, the room temperature-storable substrate solution comprising the at least one caged substrate compound;
adding an aliquot of the sample to the assay vessel;
thereafter, transferring an aliquot of the room temperature-storable substrate solution from the substrate storage container into the assay vessel, and mixing the assay composition contained therein;
incubating the assay composition in the assay vessel for the incubation period; and
detecting the light emitted from the assay composition through the walls of the assay vessel with an optoelectronic device.

In accordance with a second aspect the invention provides a system for detecting an extracellular analyte in a sample, comprising: a biosensor system selected from (i) an extracellular bioluminescent sensor molecule comprising a donor lumiphore, an analyte-sensitive domain and an acceptor fluorophore; and (ii) a precursor system thereof, the precursor system comprising a pair of molecules adapted to react in solution to form the extracellular bioluminescent sensor molecule, the biosensor system contained in an assay vessel configured to receive the sample for combination with the biosensor system in an assay composition; a room temperature-stable substrate solution comprising at least one caged substrate compound, for dispensing into the assay composition in the assay vessel, the substrate solution contained in a substrate storage vessel; and an optoelectronic device configured to detect light emitted from the assay composition through the walls of the assay vessel, wherein the ratio of luminosity associated with the acceptor fluorophore to luminosity associated with the donor lumiphore indicates an interaction of the extracellular analyte with the analyte-sensitive domain, wherein the at least one caged substrate compound is adapted to uncage in the assay composition, thereby producing an uncaged substrate compound which energises the donor lumiphore.

According to some embodiments, the optoelectronic device is configured to receive the assay vessel and to incubate the assay composition contained therein for an incubation period sufficient for the extracellular analyte, if present in the sample, to interact with the analyte-sensitive domain. The optoelectronic device may be configured to incubate the assay composition at a predetermined incubation temperature.

According to some embodiments, the biosensor system is present in a solid composition. The solid composition may be a freeze-dried solid composition. The solid composition may further comprise an uncaging agent for uncaging the at least one caged substrate compound in the assay composition.

According to some embodiments, the substrate solution comprises at least two different caged substrate compounds adapted to uncage in the assay composition to produce the uncaged substrate compound at different reaction rates.

According to some embodiments, each caged substrate compound is a caged coelenterazine compound comprising a carbonyl protecting group at the 3′-position.

According to some embodiments, each caged substrate compound has a structure according to Formula 1 or Formula 2:

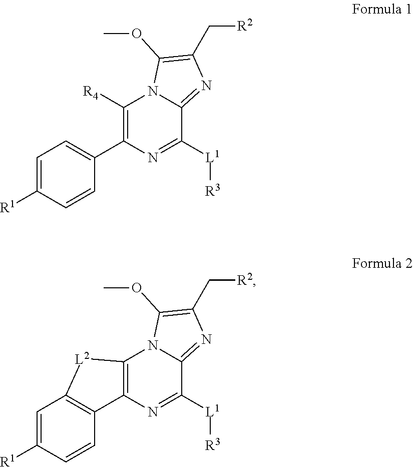

wherein: each R¹ is independently selected from H, OH, OR^{alk} where R^{alk} is C₁-C₆ alkyl, and NH₂, each R² is independently selected from H, Ar, and (CH₂)ₙAr, where each Ar is independently a carbocyclic aromatic group optionally substituted with OH, C₁-C₆ alkyl or halogen, and n is 1 or 2, each L¹ is independently selected from CH₂ or S, each R³ is independently selected from C₆H₅, CH₂C₆H₅, C₁-C₆ alkyl, and C₁-C₆ cycloalkyl, R⁴ is independently selected from H and CH₃, L² is independently selected from (CH₂)ₙ where n is 1-3 and CH═CH, and each X is independently a carbonyl protecting group.

According to some embodiments, each X has a structure according to Formula 3, Formula 4 or Formula 5:

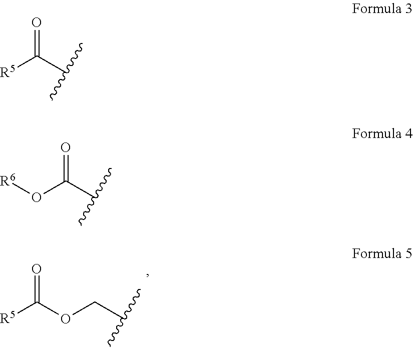

wherein R⁵, R⁶ and R⁷ are each independently optionally substituted C₁-C₁₂ alkyl, C₁-C₁₂ cycloalkyl or C₁-C₁₂ aryl.

According to some embodiments, the uncaged substrate compound is a coelenterazine compound. The coelenterazine compound may be bisdeoxycoelenterazine.

According to some embodiments, the substrate solution is a non-aqueous solution comprising a water-miscible aprotic solvent, for example DMSO.

According to some embodiments, the substrate storage vessel is configured to dispense an aliquot of the substrate solution into the assay vessel. The substrate storage vessel may be configured to dispense the substrate solution drop-wise into the assay vessel.

According to some embodiments, the extracellular bioluminescent sensor molecule is a BRET² biosensor.

According to some embodiments, the optoelectronic device comprises a first light detector comprising a micro-photon multiplier tube fitted with a band pass filter to selectively permit transmission of light wavelengths associated with the acceptor fluorophore and a second light detector comprising a micro-photon multiplier tube fitted with a band pass filter to selectively permit transmission of light wavelengths associated with the donor lumiphore.

According to some embodiments, the optoelectronic device is configured to incubate the assay composition at a predetermined incubation temperature.

In accordance with a third aspect the invention provides a BRET reagent system for combination with a sample to produce a bioluminescent assay composition having a BRET ratio, after incubation of the assay composition, that is indicative of an extracellular analyte in the sample, the BRET reagent system comprising: (a) a solid composition comprising a biosensor system selected from (i) an extracellular bioluminescent sensor molecule comprising a donor lumiphore, an analyte-sensitive domain susceptible to interaction with the extracellular analyte, and an acceptor fluorophore; and (ii) a precursor system thereof, the precursor system comprising a pair of molecules adapted to react in solution to form the extracellular bioluminescent sensor molecule, wherein the solid composition is dispersible in a liquid comprising the sample to produce the assay composition; and (b) a room temperature-stable substrate solution comprising at least one caged substrate compound, for dispensing into the assay composition, wherein the at least one caged substrate compound is adapted to uncage in the assay composition to produce an uncaged substrate compound which energises the donor lumiphore.

According to some embodiments, the solid composition is contained in an assay vessel configured to receive the liquid comprising the sample and to permit transmission of light from the assay composition through the assay vessel walls for detection.

According to some embodiments, the solid composition is a freeze-dried composition prepared by freeze-drying an aqueous precursor composition in the assay vessel.

According to some embodiments, the solid composition further comprises an uncaging agent for uncaging the at least one caged substrate compound in the assay composition.

According to some embodiments, the room temperature-stable substrate solution is contained in a substrate storage vessel, the substrate storage vessel configured to dispense an aliquot of the substrate solution into the assay composition. The substrate storage vessel may be configured to dispense the substrate solution drop-wise into the assay composition.

According to some embodiments, the room temperature-stable substrate solution comprises at least two different caged substrate compounds adapted to uncage in the assay composition to produce the uncaged substrate compound at different reaction rates.

According to some embodiments, the BRET reagent system is in the form of a kit comprising instructions for use of the BRET reagent system in a method according to any embodiment of the first aspect.

In accordance with a fourth aspect the invention provides a solid biosensor composition for BRET detection of an extracellular analyte in a sample, the solid biosensor composition comprising: (a) a biosensor system selected from (i) an extracellular bioluminescent sensor molecule comprising a donor lumiphore, an analyte-sensitive domain susceptible to interaction with the extracellular analyte, and an acceptor fluorophore; and (ii) a precursor system thereof, the precursor system comprising a pair of molecules adapted to react in solution to form the extracellular bioluminescent sensor molecule; and (b) an uncaging agent, wherein the solid biosensor composition is dispersible in a liquid comprising the sample to produce an assay composition, and wherein the uncaging agent is adapted to uncage at least one caged substrate compound in the assay composition, thereby producing an uncaged substrate compound which energises the donor lumiphore.

In accordance with a fifth aspect the invention provides a substrate dispensing system for BRET detection of an extracellular analyte in a sample, the substrate dispensing system comprising a room temperature-stable substrate solution comprising at least one caged substrate compound, wherein the substrate solution is contained in a substrate storage vessel configured to dispense a pre-determined aliquot of the substrate solution into an assay composition comprising the sample and a BRET bioluminescent sensor molecule.

In some embodiments, the room temperature-stable substrate solution comprises at least two different caged substrate compounds adapted to uncage at different reaction rates.

In accordance with a sixth aspect the invention provides a substrate composition for BRET detection of an extracellular analyte in a sample, the substrate composition comprising at least two different caged substrate compounds adapted to uncage in an assay composition comprising the sample and a BRET bioluminescent sensor molecule, thereby producing an uncaged substrate compound at different reaction rates.

Where the terms “comprise”, “comprises” and “comprising” are used in the specification (including the claims) they are to be interpreted as specifying the stated features, integers, steps or components, but not precluding the presence of one or more other features, integers, steps or components, or group thereof.

Further aspects of the invention appear below in the detailed description of the invention.

Any embodiment herein shall be taken to apply mutatis mutandis to any other embodiment unless specifically stated otherwise. In a non-limiting example, other embodiments of the third, fourth, fifth, sixth and other aspects may comprise features as disclosed herein in the context of the first or second aspect.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of 20 compositions of matter.

### BRIEF DESCRIPTION OF DRAWINGS

Embodiments of the invention will herein be illustrated by way of example only with reference to the accompanying drawings in which:

FIG. 1 schematically depicts an assay vessel comprising a light-emitting assay composition, held in the light-detection portion of an optoelectronic device according to some embodiments of the invention.

FIG. 2 schematically depicts an optoelectronic device according to some embodiments of the invention, comprising the light-detection portion of FIG. 1.

FIG. 3 is a graph showing the acceptor fluorophore luminosity of analyte-free BRET² assays energised with uncaged bisdeoxycoelenterazine substrate (CLZ400a in ethanol), as a function of the substrate solution storage time at different temperatures (Example 1).

FIG. 4 is a graph showing the BRET² ratio of analyte-free BRET² assays energised with uncaged bisdeoxycoelenterazine substrate (CLZ400a in ethanol), as a function of the substrate solution storage time at different temperatures (Example 1).

FIG. 5 is a graph showing the acceptor fluorophore luminosity of analyte-free BRET² assays energised with uncaged bisdeoxycoelenterazine substrate (CLZ400a-HCl in DMSO), as a function of the substrate solution storage time at different temperatures (Example 1).

FIG. 6 is a graph showing the BRET² ratio of analyte-free BRET² assays energised with uncaged bisdeoxycoelenterazine substrate (CLZ400a-HCl in DMSO), as a function of the substrate solution storage time at different temperatures (Example 1).

FIG. 7 is a graph showing the acceptor fluorophore luminosity of analyte-free BRET² assays energised by in situ decaging of caged bisdeoxycoelenterazine compound 7, as a function of the substrate solution storage time at different temperatures (Example 1).

FIG. 8 is a graph showing the BRET² ratio of analyte-free BRET² assays energised by in situ decaging of caged bisdeoxycoelenterazine compound 7, as a function of the substrate solution storage time at different temperatures (Example 1).

FIG. 9 is a graph showing the acceptor fluorophore luminosity of analyte-free BRET² assays energised by in situ decaging of caged bisdeoxycoelenterazine compound 8, as a function of the substrate solution storage time at different temperatures (Example 1).

FIG. 10 is a graph showing the BRET² ratio of analyte-free BRET² assays energised by in situ decaging of caged bisdeoxycoelenterazine compound 8, as a function of the substrate solution storage time at different temperatures (Example 1).

FIG. 11 is a graph showing the acceptor fluorophore luminosity of analyte-free BRET² assays energised by in situ decaging of caged bisdeoxycoelenterazine compound 10, as a function of the substrate solution storage time at different temperatures (Example 1).

FIG. 12 is a graph showing the BRET² ratio of analyte-free BRET² assays energised by in situ decaging of caged bisdeoxycoelenterazine compound 10, as a function of the substrate solution storage time at different temperatures (Example 1).

FIG. 13 is a graph showing the acceptor fluorophore luminosity of analyte-free BRET² assays energised by in situ decaging of a mixture of caged bisdeoxycoelenterazine compound 8 and 10, as a function of the substrate solution storage time at different temperatures (Example 1).

FIG. 14 is a graph showing the BRET² ratio of analyte-free BRET² assays energised by in situ decaging of a mixture of caged bisdeoxycoelenterazine compound 8 and 10, as a function of the substrate solution storage time at different temperatures (Example 1).

FIG. 15 is a graph showing the acceptor fluorophore luminosity of analyte-free BRET² assays comprising caged bisdeoxycoelenterazine compounds 7, 8 or 10 in a buffer matrix, without decaging agent or with added porcine liver esterase (Example 2).

FIG. 16 is a graph showing the acceptor fluorophore luminosity of analyte-free BRET² assays comprising caged bisdeoxycoelenterazine compounds 7, 8 or 10 in a milk matrix, without extraneous decaging agent or with added porcine liver esterase (Example 2).

FIG. 17 is a graph showing the acceptor fluorophore luminosity of analyte-free BRET² assays comprising different mixtures of caged bisdeoxycoelenterazine compounds and different amounts of porcine liver esterase compounds in a milk matrix (Example 2; Table 9).

FIG. 18 is a graph showing the BRET² ratio of analyte-free BRET² assays comprising different mixtures of caged bisdeoxycoelenterazine compounds and different amounts of porcine liver esterase compounds in a milk matrix (Example 2; Table 9).

FIG. 19 is a graph showing the acceptor fluorophore luminosities as shown in FIG. 17, each normalised to 100% and depicted only during the window where the luminosity was 80-100% of maximum.

FIG. 20 is a graph showing the maximum acceptor fluorophore luminosities of the assays presented in FIG. 17, normalised relative to the assay of maximum peak luminosity and depicting the window where the luminosity was 80-100% of maximum.

FIG. 21 is a graph showing the BRET² ratio standard deviation of the assays presented in FIG. 17, during the window where the luminosity was 80-100% of maximum.

FIG. 22 is a graph showing the acceptor fluorophore luminosity of analyte-free BRET² assays energised by in situ decaging of caged bisdeoxycoelenterazine compounds 8 and 10 with 1 U porcine liver esterase, as a function of the storage time of the freeze-dried BRET² biosensor composition at different temperatures (Example 3).

FIG. 23 is a graph showing the BRET² ratio of analyte-free BRET² assays energised by in situ decaging of caged bisdeoxycoelenterazine compounds 8 and 10 with 1 U porcine liver esterase, as a function of the storage time of the freeze-dried BRET² biosensor composition at different temperatures (Example 3).

FIG. 24 is a graph showing the acceptor fluorophore luminosity of analyte-free BRET² assays comprising caged bisdeoxycoelenterazine compound 10 and added porcine liver esterase in raw milk, UHT milk and fresh (pasteurised) milk matrices (Example 4).

FIG. 25 is a graph showing the acceptor fluorophore luminosity of analyte-free BRET² assays comprising caged bisdeoxycoelenterazine compound 10 and added porcine liver esterase in raw milk, heat-treated raw milk and sonicated raw milk matrices (Example 4).

FIG. 26 is a graph showing the acceptor fluorophore luminosity of analyte-free BRET² assays comprising caged bisdeoxycoelenterazine compound 10 and different amounts of porcine liver esterase in a UHT milk matrix (Example 4).

FIG. 27 is a graph showing the BRET² ratio as a function of acceptor fluorophore luminosity during an analyte-free BRET² assay comprising caged bisdeoxycoelenterazine compound 10 and added porcine liver esterase in raw milk matrix (Example 4).

FIG. 28 is a calibration plot of BRET² ratios obtained for 10 and 15 minute BRET² assays (caged bisdeoxycoelenterazine compound 10 uncaged in situ by porcine liver esterase) of raw milk samples spiked with known amounts of AprX (Example 5).

FIG. 29 is a calibration plot of BRET² ratios obtained for 10 and 15 minute BRET² assays (caged bisdeoxycoelenterazine compound 10 uncaged in situ by porcine liver esterase) of UHT milk samples spiked with known amounts of AprX (Example 5).

FIG. 30 illustrates an example sample preparation station to prepare milk analysis samples. A provides an overview of the preparation station. B is in image of an example dropper bottle holder storage space located within an air-tight cavity. C is a graph illustrating dispensed volume of DMSO (1 mg≙1 μL) if the dropper bottle is operated via its holder system or if it is operated by hand (20 repeats). D is a graph illustrating dispensed volume of full cream milk (1 mg≙1 μL) using the dispensette or a 1 mL fixed volume pipette (20 repeats). E is a graph showing the coefficients of variation for measurements shown in C and D. F and G graph of the average maximum signals for the blue and green channel (F) and BRET ratios (G) on the optoelectronic device, comparing FD-B4 (1 U/assay esterase) reagents prepared with UHT full cream milk on the preparation station or by pipette (3 repeats). H and I show the results of a stability trial of compound-10 substrate (4.3 mM in dry DMSO) stored in the preparation station dropper bottle holder cavity (3 repeats) over 16 weeks at room temperature (Example 6).

FIG. 31 illustrates development of a lactose-sensing assay on the optoelectronic device using a freeze-dried BRET²-lactose sensor. A is a graph showing CLARIOstar plate reader analysis of the wild-type lactose-sensor and three sensor variants. B is a calibration plot for the lactose sensor (wild-type and variant) in PBS buffer supplemented with serial dilutions of lactose. C is a graph showing green/blue channel bioluminescence counts of freeze-dried lactose biosensor reconstituted with 10% lactose-free milk (diluted in dH₂O) and supplemented with compound-10 substrate, analysed on the optoelectronic device. D is a graph of BRET ratios of freeze-dried lactose biosensor reconstituted with 10% lactose-free milk (diluted in dH₂O) and supplemented with compound-10 substrate, analysed on the optoelectronic device. E is a graph showing BRET ratio analysis of various freeze-dried lactose sensor formulations on the optoelectronic device. F is a graph showing BRET ratio difference between lactose-free milk samples with or without added 5% (w/v) lactose analysed on the optoelectronic device using various freeze-dried lactose sensor formulations. BRET ratio end points were taken at 1 min intervals. G shows optoelectronic device analysis (BRET ratios) of lactase-treated lactose-free milk with or without added 5% (w/v) lactose using FD-L3-13 reagents in a 4 min assay (three replicates). H is a calibration plot of lactose sensing of lactase-treated lactose-free milk samples supplemented with serial dilutions of lactose (two replicates). (Example 7).

FIG. 32 is a plot of BRET ratios of freeze dried plasmin sensor reconstituted with raw milk and supplemented with compound-10 substrate, analysed on the optoelectronic device (Example 8)

FIG. 33 illustrates analysis of raw milk samples using the optoelectronic device and FD-Plasminogen-1 assay reagents. A is a plot of BRET Ratios over 900 sec for raw milk or raw milk heat-treated for 20 min at 90°. B is a graph of the BRET ratio from a 15-min assay for raw milk and heat-treated raw milk. C is a graph of the BRET ratio from a 15-min assay for raw milk and heat-treated raw milk samples spiked with 3-100 mU/mL human plasminogen (Example 9).

FIG. 34 illustrates analysis of UHT milk samples spiked with subtilisin using FD-B4 (5 unit esterase per assay) and the optoelectronic device. A is a graph of BRET ratio results from a 10-min and 15-min assay using UHT milk spiked with 0, 100 or 1000 μU/mL subtilisin. B is a plot of BRET ratios over 800 sec for UHT milk samples spiked with 500 μU/mL subtilisin or 1000 μU/mL AprX. Samples were either left untreated before analysis or supplemented with 0.8 mM PMSF and incubated on ice for 30 min prior to analysis (Example 10).

FIG. 35 illustrates Micro Photon Multiplier Tube (μPMT) calibration of the optoelectronic device using gaseous tritium light sources (GTLS). A shows luminescent spectra of TrigaLight® GTLS Micromarkers (UV Blue, White and Green). The horizontal lines indicate the optoelectronic device filters for ‘Blue’ and ‘Green’ photons. B is a plot of ‘Blue’ and ‘Green’ channel photon counts of a TrigaLight® placed at the upper end of the tube holder in the optoelectronic device. C is a plot of blue counts of four runs of a TrigaLight® placed at the bottom of a 2 mL tube on the optoelectronic device (2 min at 37° C.). The tube was removed and reinserted into the tube holder after each run. D shows ‘Green’ and ‘Blue’ channel signal counts over 20 min at various temperatures. G shows CT² measurements of three optoelectronic devices (B0001-B0003) using the same calibration tool (Example 11).

FIG. 36 shows an example schematic drawing of the Cybertongue calibration tool (CT²). While not shown, the screw cap form can also include a lug fitting a notch located at the reciprocal of the optoelectronic device, thus increasing the reproducibility of calibration measurements by ensuring that the tool is placed at the same location for every run (Example 11).

FIG. 37 shows an example schematic drawing of the Cybertongue calibration tool (CT²) without the positioning notch and lug (Example 11).

Key to sequence listing

| | |
|---|---|
| SEQ ID NO: 1 | Amino acid sequence of AprX biosensor. |
| | MVSKGEELFTGVVPILVELDGDVNGHKFSVSGEGEGDATYGKLTLKFICTTGKLPVPW |
| | PTLVTTLSYGVQCFSRYPDHMKQHDFFKSAMPEGYVQERTIFFKDDGNYKTRAEVKFE |
| | GDTLVNRIELKGIDFKEDGNILGHKLEYNYNSHNVYIMADKQKNGIKVNFKIRHNIED |
| | GSVQLADHYQQNTPIGDGPVLLPDNHYLSTQSALSKDPNEKRDHMVLLEFVTAAGITL |
| | GMDELYKLQGSEMKKNQNTEIGSFEMASKVYDPEQRKRMITGPQWWARCKQMNVLDSF |
| | INYYDSEKHAENAVIFLHGNAASSYLWRHVVPHIEPVARCIIPDLIGMGKSGKSGNGS |
| | YRLLDHYKYLTAWFELLNLPKKIIFVGHDWGAALAFHYSYEHQDKIKAIVHAESVVDV |
| | IESWDEWPDIEEDIALIKSEEGEKMVLENNFFVETVLPSKIMRKLEPEEFAAYLEPFK |
| | EKGEVRRPTLSWPREIPLVKGGKPDVVQIVRNYNAYLRASDDLPKMFIESDPGFFSNA |
| | IVEGAKKFPNTEFVKVKGLHFSQEDAPDEMGKYIKSFVERVLKNEQ |
| SEQ ID NO: 2 | Amino acid sequence of LacB1 (GFP²-BgaR-RLuc8 fusion protein). |
| | MRGSHHHHHHGMASMTGGQQMGRDLYDDDDKDRWGSEFMVSKGEELFTGVVPILVELD |
| | GDVNGHKFSVSGEGEGDATYGKLTLKFICTTGKLPVPWPTLVTTLSYGVQCESRYPDH |
| | MKQHDFFKSAMPEGYVQERTIFFKDDGNYKTRAEVKFEGDTLVNRIELKGIDFKEDGN |
| | ILGHKLEYNYNSHNVYIMADKQKNGIKVNFKIRHNIEDGSVQLADHYQQNTPIGDGPV |
| | LLPDNHYLSTQSALSKDPNEKRDHMVLLEFVTAAGITLGMDELYKLQMQILWKKYVKE |
| | NFEMNVDECGIEQGIPGLGYNYEVLKNAVIHYVTKGYGTFKFNGKVYNLKQGDIFILL |
| | KGMQVEYVASIDDPWEYYWIGFSGSNANEYLNRTSITNSCVANCEENSKIPQIILNMC |
| | EISKTYNPSRSDDILLLKELYSLLYALIEEFPKPFEYKDKELHTYIQDALNFINSNYM |
| | HSITVQEIADYVNLSRSYLYKMFIKNLGISPQRYLINLRMYKATLLLKSTKLPIGEVA |
| | SSVGYSDSLLFSKTFSKHFSMSPLNYRNNQVNKPSIFELQMASKVYDPEQRKRMITGP |
| | QWWARCKQMNVLDSFINYYDSEKHAENAVIFLHGNATSSYLWRHVVPHIEPVARCIIP |
| | DLIGMGKSGKSGNGSYRLLDHYKYLTAWFELLNLPKKIIFVGHDWGAALAFHYAYEHQ |
| | DRIKAIVHMESVVDVIESWDEWPDIEEDIALIKSEEGEKMVLENNFFVETVLPSKIMR |
| | KLEPEEFAAYLEPFKEKGEVRRPTLSWPREIPLVKGGKPDVVQIVRNYNAYLRASDDL |
| | PKLFIESDPGFFSNAIVEGAKKFPNTEFVKVKGLHFLQEDAPDEMGKYIKSFVERVLK |
| | NEQ) |
| SEQ ID NO: 3 | Amino acid sequence of LacB6 (GFP²-BgaR(D140A/1141A)-RLuc8 |
| | fusion protein). |
| | MRGSHHHHHHGMASMTGGQQMGRDLYDDDDKDRWGSEFMVSKGEELFTGVVPILVELD |
| | GDVNGHKFSVSGEGEGDATYGKLTLKFICTTGKLPVPWPTLVTTLSYGVQCFSRYPDH |
| | MKQHDFFKSAMPEGYVQERTIFFKDDGNYKTRAEVKFEGDTLVNRIELKGIDFKEDGN |
| | ILGHKLEYNYNSHNVYIMADKQKNGIKVNFKIRHNIEDGSVQLADHYQQNTPIGDGPV |
| | LLPDNHYLSTQSALSKDPNEKRDHMVLLEFVTAAGITLGMDELYKLQMQILWKKYVKE |
| | NFEMNVDECGIEQGIPGLGYNYEVLKNAVIHYVTKGYGTFKFNGKVYNLKQGDIFILL |
| | KGMQVEYVASIDDPWEYYWIGFSGSNANEYLNRTSITNSCVANCEENSKIPQIILNMC |
| | EISKTYNPSRSDAALLLKELYSLLYALIEEFPKPFEYKDKELHTYIQDALNFINSNYM |
| | HSITVQEIADYVNLSRSYLYKMFIKNLGISPQRYLINLRMYKATLLLKSTKLPIGEVA |
| | SSVGYSDSLLFSKTFSKHFSMSPLNYRNNQVNKPSIFELQMASKVYDPEQRKRMITGP |
| | QWWARCKQMNVLDSFINYYDSEKHAENAVIFLHGNATSSYLWRHVVPHIEPVARCIIP |
| | DLIGMGKSGKSGNGSYRLLDHYKYLTAWFELLNLPKKIIFVGHDWGAALAFHYAYEHQ |
| | DRIKAIVHMESVVDVIESWDEWPDIEEDIALIKSEEGEKMVLENNFFVETVLPSKIMR |
| | KLEPEEFAAYLEPFKEKGEVRRPTLSWPREIPLVKGGKPDVVQIVRNYNAYLRASDDL |
| | PKLFIESDPGFFSNAIVEGAKKFPNTEFVKVKGLHFLQEDAPDEMGKYIKSFVERVLK |
| | NEQ |
| SEQ ID NO: 4 | Amino acid sequence of LacB13 (GFP²-BgaR(N12S/D140S/I141A)- |
| | RLuc8 fusion protein). |
| | MRGSHHHHHHGMASMTGGQQMGRDLYDDDDKDRWGSEFMVSKGEELFTGVVPILVELD |
| | GDVNGHKFSVSGEGEGDATYGKLTLKFICTTGKLPVPWPTLVTTLSYGVQCFSRYPDH |
| | MKQHDFFKSAMPEGYVQERTIFFKDDGNYKTRAEVKFEGDTLVNRIELKGIDFKEDGN |
| | ILGHKLEYNYNSHNVYIMADKQKNGIKVNFKIRHNIEDGSVQLADHYQQNTPIGDGPV |
| | LLPDNHYLSTQSALSKDPNEKRDHMVLLEFVTAAGITLGMDELYKLQMQILWKKYVKE |
| | SFEMNVDECGIEQGIPGLGYNYEVLKNAVIHYVTKGYGTFKFNGKVYNLKQGDIFILL |
| | KGMQVEYVASIDDPWEYYWIGFSGSNANEYLNRTSITNSCVANCEENSKIPQIILNMC |
| | EISKTYNPSRSDSALLLKELYSLLYALIEEFPKPFEYKDKELHTYIQDALNFINSNYM |
| | HSITVQEIADYVNLSRSYLYKMFIKNLGISPQRYLINLRMYKATLLLKSTKLPIGEVA |
| | SSVGYSDSLLFSKTFSKHFSMSPLNYRNNQVNKPSIFELQMASKVYDPEQRKRMITGP |
| | QWWARCKQMNVLDSFINYYDSEKHAENAVIFLHGNATSSYLWRHVVPHIEPVARCIIP |
| | DLIGMGKSGKSGNGSYRLLDHYKYLTAWFELLNLPKKIIFVGHDWGAALAFHYAYEHQ |
| | DRIKAIVHMESVVDVIESWDEWPDIEEDIALIKSEEGEKMVLENNFFVETVLPSKIMR |
| | KLEPEEFAAYLEPFKEKGEVRRPTLSWPREIPLVKGGKPDVVQIVRNYNAYLRASDDL |
| | PKLFIESDPGFFSNAIVEGAKKFPNTEFVKVKGLHFLQEDAPDEMGKYIKSFVERVLK |
| | NEQ |
| SEQ ID NO: 5 | Amino acid sequence of LacB16 (GFP²-BgaR(D140S/I141A/L142A/ |
| | L144A)-RLuc8 fusion protein). |
| | MRGSHHHHHHGMASMTGGQQMGRDLYDDDDKDRWGSEFMVSKGEELFTGVVPILVELD |
| | GDVNGHKFSVSGEGEGDATYGKLTLKFICTTGKLPVPWPTLVTTLSYGVQCFSRYPDH |
| | MKQHDFFKSAMPEGYVQERTIFFKDDGNYKTRAEVKFEGDTLVNRIELKGIDFKEDGN |
| | ILGHKLEYNYNSHNVYIMADKQKNGIKVNFKIRHNIEDGSVQLADHYQQNTPIGDGPV |
| | LLPDNHYLSTQSALSKDPNEKRDHMVLLEFVTAAGITLGMDELYKLQMQILWKKYVKE |
| | NFEMNVDECGIEQGIPGLGYNYEVLKNAVIHYVTKGYGTFKFNGKVYNLKQGDIFILL |
| | KGMQVEYVASIDDPWEYYWIGESGSNANEYLNRTSITNSCVANCEENSKIPQIILNMC |
| | EISKTYNPSRSDSAALAKELYSLLYALIEEFPKPFEYKDKELHTYIQDALNFINSNYM |
| | HSITVQEIADYVNLSRSYLYKMFIKNLGISPQRYLINLRMYKATLLLKSTKLPIGEVA |
| | SSVGYSDSLLFSKTFSKHFSMSPLNYRNNQVNKPSIFELQMASKVYDPEQRKRMITGP |
| | QWWARCKQMNVLDSFINYYDSEKHAENAVIFLHGNATSSYLWRHVVPHIEPVARCIIP |
| | DLIGMGKSGKSGNGSYRLLDHYKYLTAWFELLNLPKKIIFVGHDWGAALAFHYAYEHQ |
| | DRIKAIVHMESVVDVIESWDEWPDIEEDIALIKSEEGEKMVLENNFFVETVLPSKIMR |
| | KLEPEEFAAYLEPFKEKGEVRRPTLSWPREIPLVKGGKPDVVQIVRNYNAYLRASDDL |
| | PKLFIESDPGFFSNAIVEGAKKFPNTEFVKVKGLHFLQEDAPDEMGKYIKSFVERVLK |
| | NEQ |

### DETAILED DESCRIPTION

### General Techniques and Definitions

Unless specifically defined otherwise, all technical and scientific terms used herein shall be taken to have the same meaning as commonly understood by one of ordinary skill in the art (e.g., in chemiluminescent based sensor technology-particularly bioluminescent based sensor technology, molecular biology, protein chemistry, bioconjugation techniques, biochemistry and the like). Unless otherwise indicated, the recombinant protein, cell culture, bioconjugation, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et ah, Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et ah, (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et ah, (editors) Current Protocols in Immunology, John Wiley & Sons (including all updates until present), Greg T. Hermanson, Bioconjugate Techniques. Elsevier Inc. (2013).

The term “and/or”, e.g., “X and/or Y” shall be understood to mean either “X and Y” or “X or Y” and shall be taken to provide explicit support for both meanings or for either meaning.

Unless the context suggests otherwise, the mention of a term in singular such as sensor and substrate clearly means the plural as well. For instance, logically many individual molecules will be flowed through the device or contained within a well rather than a single molecule.

As used herein, the term about, unless stated to the contrary, refers to +/−10%, more preferably +/−5%, even more preferably +/−1%, of the designated value.

### Methods for Detecting an Extracellular Analyte

The present invention relates to a method for detecting an extracellular analyte in a sample. In some examples, the method comprises incubating an assay composition comprising the sample and a biosensor system selected from (i) an extracellular bioluminescent sensor molecule comprising a donor lumiphore, an analyte-sensitive domain and an acceptor fluorophore and (ii) a precursor system thereof comprising a pair of molecules adapted to react in solution to form the extracellular bioluminescent sensor molecule. The assay composition is incubated for an incubation period sufficient for the extracellular analyte, if present in the sample, to interact with the analyte-sensitive domain. At least one caged substrate compound is uncaged in the assay composition during the incubation period to produce an uncaged substrate compound which energises the donor lumiphore for the subsequent detection step. Light emitted from the assay composition, including emissions associated with the acceptor fluorophore and with the donor lumiphore, is then detected after the incubation period. The ratio of luminosity (i.e. intensity) associated with the acceptor fluorophore to luminosity (i.e. intensity) associated with the donor lumiphore indicates an interaction of the extracellular analyte with the analyte-sensitive domain.

### Sample and Extracellular Analyte

The sample may be any sample which contains, or may contain, an extracellular analyte of interest or concern. The methods disclosed herein are of particular interest for measuring the activity of enzymes or the concentration of a wide range of analytes in complex biological matrices. In some embodiments, therefore, the sample is a food or beverage composition, including food or beverage intermediates, products and derivatives. For example, a food or beverage derivative may be an aqueous extract or other worked-up aqueous sample derived from a food or beverage composition for the purposes of analysis. In some embodiments, the sample is a dairy composition. For example, it may be a sample of raw or treated milk.

The milk may be cow's milk, goat's milk, sheep's milk, buffalo milk, camel's milk or human milk. The milk may be coloured and flavoured including, for example vanilla, chocolate, coffee or strawberry milk, it may be lactose modified such as “low lactose” or “lactose free” and it may be sweetened or unsweetened. The sample may be whey or be reconstituted from skim or whole milk powder, or whey powder, by adding water. It may also be a milk-based product that contains other ingredients designed to provide a meal or snack replacement. The sample may be a fermentation or plant-based product sold as a substitute for milk, including those containing casein and or whey proteins prepared by fermentation or made from soy, almonds, oats, coconut, etc.

The sample may be another beverage, including fruit juices, cordials, beer, wine or spirits or coffee, tea etc. The sample may be a soup, sauce or condiment. The sample may also be a thickened product, or extract or dilution thereof, such as yoghurt or custard. The sample may also be an extract of almost any dried or semi-solid food, made from or including grains, meat, bakery products, chocolate, meat, eggs, fish, cheese, nuts, fruits, herbs, spices, snack foods etc. In such cases, the sample may be prepared by extracting the food with an aqueous buffer with or without mechanical grinding and may include the addition of detergents, emulsifying agents and enzymes to aid the extraction of analytes.

The sample may be an extract or preparation of a prescription or non-prescription pharmaceutical. The sample may be an extract or preparation of a cosmetic or personal care product.

The sample may be a biological fluid from a human or other animal, including blood, serum, plasma, tears, saliva, urine, faeces, cerebrospinal fluid, lymph, condensed expired breath fluid, etc. The sample may be an extract or preparation of a solid tissue sample from a human or other animal that has been ground or homogenised to provide a liquid sample. The sample may be an extract of a plant, including sap or ground leaf or other plant tissues. The sample may be a culture medium for mammalian or other animal cells or tissues or for plant cells or tissues or a fermentation medium for bacteria, fungi or other microorganisms.

The sample may be an environmental sample, including wash water, waste water, surface water, ground water, tap water, rainwater, leachate or an aqueous extract of a soil sample. The sample may be an environmental sample collected by swabbing natural or man-made surfaces, including indoor surfaces, and making an aqueous rinse extract from the swab. For example, the sample may be prepared by swabbing a surface with a wetted swab (e.g. cotton bud) and making an aqueous rinse extract from the swab. The sample may also be prepared by equilibrating air with an aqueous buffer and testing the buffer, for example using a wet-walled cyclone apparatus (such as SASS 2400) or misting apparatus. The sample may be any ingredient or raw material used in a manufacturing process, including food and beverage manufacturing, or an aqueous extract thereof.

The sample may be a liquid or a solid. In some embodiments, the sample is a liquid, and in particular an aqueous liquid.

The methods disclosed herein are applicable to the detection of a wide range of extracellular analytes. In some embodiments, the extracellular analytes include, but not limited to, extracellular analytes for which BRET-based biosensing systems have been previously reported. In some embodiments, the extracellular analytes, include, but not limited, to extracellular analytes for which chemiluminescent biosensing systems have been previously reported

As used herein, an extracellular analyte is a molecule of interest, and includes extracellular molecules (e.g. compounds including sugars, lipids, and proteins, and intermediary metabolites, including those that are derived from cells, tissues or organs, and secreted/transported/shed from cells, tissues or organs), as well as intracellular molecules that have become extracellular as a result of disruption to a cell (e.g. by cell death). The term extracellular analyte does not include molecules that are detected in a cell that also comprises a BRET reporter expressed in the cell.

Examples of suitable extracellular analytes which may be detected by the methods disclosed herein include:
disaccharides and monosaccharides, including glucose, sucrose, lactose, maltose and the like, and derivatives thereof (e.g. lactulose).
oligopeptides, polypeptides and proteins, including food allergens, nutritional proteins such as whey proteins, caseins, gluten and peptides from these
linear and conformational epitopes (e.g. beta casein A1/A2 dimorphism)·
other antigens
other proteins, including those that are validated health biomarkers such as ferritin, haemoglobin A1c
antibiotics of all major classes
mycotoxins such as aflatoxins, fumonisins, patulin and the like.
heavy metals, including lead, zinc, cadmium, arsenic, etc.
other toxins, including cyclic peptides (e.g. cereulide)
adenosine triphosphate
other enzymes such as Alkaline phosphatase, Esterase
amino acids and dipeptides
hormones, including steroid hormones such as cortisol, testosterone, oestrogen, progesterone and derivatives thereof
peptide hormones, including follicle stimulating hormones, thyroid stimulating hormones,
anions as used in food preservation such as nitrite, nitrate, benzoate, sulphite, propionate, sorbate
high intensity sweeteners
spice active compounds such as curcumin, capsaicin and sanshools
therapeutic pharmaceuticals
vitamins and vitamin precursors such as Vitamin B12 and 1a,25(OH)₂-Vitamin D3 and its biological precursors

In some embodiments, the extracellular analyte is selected from the group consisting of an enzyme, a zymogen and a carbohydrate. In some embodiments, the extracellular analyte is selected from the group consisting of an enzyme and a carbohydrate. In some embodiments, the enzyme is a protease or a zymogen. In some embodiments, the enzyme is a protease.

In some embodiments, the extracellular analyte is a protease, for example alkaline metalloprotease (AprX), another metalloprotease or other protease, including Plasmin, Thrombin, Caspases (e.g. Caspase III), Bacillus serine proteases such as Subtilisin, and many others.

Proteases are often produced as an inactive precursor (also referred to as a proenzyme or zymogen). A zymogen or proenzyme is a protein that displays no catalytic activity but that can be processed (e.g. by proteolytic processing) into an active enzyme. In some embodiments, the extracellular analyte is a proenzyme or zymogen, for example plasminogen, prothrombin, caspase zymogen and many others. The proenzyme or zymogen can be converted to an active enzyme which can be analysed using the methods described herein. As would be understood by the person skilled in the art, a protease includes an activated zymogen. For example, plasminogen can be activated to form plasmin, which can then be detected/analysed using the methods described herein.

In some exemplary embodiments, the extracellular analyte is alkaline metalloprotease (AprX) secreted by Pseudomonas spp, such as Pseudomonas fluorescens. This protease is economically significant because it causes spoilage of otherwise acceptable quality ultra-heat-treated (UHT) milk and other dairy products. Even very low levels of this protease remaining after UHT processing are potentially detrimental.

In some exemplary embodiments, the extracellular analyte is plasmin. Plasmin is found in both human and bovine milk. Plasmin in bovine milk has been shown to degrade β-, αₛ₁- and αₛ₂-caseins to peptides. Like AprX, this protease is economically significant because it causes spoilage of otherwise acceptable quality ultra-heat-treated (UHT) milk and other dairy products. Even very low levels of this protease remaining after UHT processing are potentially detrimental.

In some embodiments, the extracellular analyte is plasminogen. Plasminogen is the inactive precursor of plasmin and is processed to form plasmin. Both urokinase-type plasminogen activator and tissue-type plasminogen activator cleave plasminogen to form plasmin. It has been disclosed that a mixture of active and inactive plasmin exists in milk. In some embodiments, plasminogen can be converted into plasmin by including an activating agent in the biosensor composition. In some embodiments, the biosensor composition comprises urokinase-type plasminogen activator or tissue-type plasminogen activator. In some embodiments, the biosensor composition comprises urokinase-type plasminogen activator. The amount of activator included in the biosensor composition should be sufficient to convert all of the zymogen to the active protein. In some embodiments, the biosensor composition comprises at least 10 U/mL, at least 100 U/mL, at least 200 U/mL, at least 300 U/mL, at least 400 U/mL, at least 500 U/mL, at least 600 U/mL, at least 700 U/mL, at least 800 U/mL, at least 900 U/mL, at least 1000 U/mL or more activator.

### Biosensor System

The biosensor system is selected from an extracellular bioluminescent sensor molecule and a precursor system thereof which comprises a pair of molecules adapted to react in solution to form the extracellular bioluminescent sensor molecule in situ. In some embodiments, the extracellular bioluminescent sensor molecule (also referred to as a BRET biosensor herein) comprises a donor lumiphore, an analyte-sensitive domain and an acceptor fluorophore. The analyte-sensitive domain may be present on a linker which holds the donor lumiphore and acceptor fluorophore in sufficiently close proximity (typically <10 nm separation) and dipole orientation to allow a Forster resonance energy transfer from the donor lumiphore to the acceptor fluorophore. When the donor lumiphore (e.g. a luciferase) acts on a suitable substrate (e.g. a luciferin), light is thus emitted from both the donor lumiphore and the acceptor fluorophore, with the relative intensities (luminosities) being highly dependent on the separation distance and relative orientations of the two moieties. Therefore, an interaction of an analyte with the analyte-sensitive domain, for example by binding or cleavage, can be detected as a change in the ratio of luminosity associated with the acceptor fluorophore to luminosity associated with the donor lumiphore (the BRET ratio). The efficiency of the (Förster) resonance energy transfer, and therefore the BRET ratio, falls off with the sixth power of the donor-acceptor separation. Appropriately designed BRET-based sensor systems are thus highly sensitive to the presence or concentration of analytes which interact with the selected analyte-sensitive domain.

The extracellular bioluminescent sensor molecule may comprise any suitable pair of compatible donor lumiphores and acceptor fluorophores, i.e. which are adapted for Förster resonance energy transfer and emit distinguishable light signals when the donor lumiphore is energised.

The donor lumiphore may be a bioluminescent protein. Examples include, but are not necessarily limited to, a luciferase, a β-galactosidase, a lactamase, a horseradish peroxidase, an alkaline phosphatase, a β-glucuronidase or a β-glucosidase. Examples of luciferases include, but are not necessarily limited to, a Renilla luciferase, a Firefly luciferase, a Coelenterate luciferase, a North American glow worm luciferase, a click beetle or other beetle luciferase, a railroad worm luciferase, a bacterial luciferase, a Gaussia luciferase, a Cypridina luciferase, or a biologically active variant or fragment of any one, or chimera of two or more, thereof.

In some embodiments, the donor lumiphore is Renilla luciferase (RLuc). Native RLuc and its variants catalyse luminescence reactions with emission spectra within the blue portion of the spectrum. The Renilla luciferase may be a variant of native Renilla luciferase with increased thermal stability, such as RLuc2 or RLuc8. In some embodiments, the donor lumiphore is RLuc2. In some embodiments, the donor lumiphore is RLuc8.

The donor lumiphore may be energised by a suitable corresponding substrate, i.e. a molecule upon which the donor lumiphore acts to generate bioluminescence. In the methods of the present disclosure, the substrate is provided in situ in the assay by uncaging a corresponding caged substrate compound, as will be described hereafter.

The acceptor fluorophore may be a protein. Examples include, but are not necessarily limited to, green fluorescent protein (GFP), blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), TagRFP, TurboFP635, red fluorescent protein (RFP), GFPS65T, Emerald, Venus, mOrange, Topaz, GFPuv, destabilised EGFP (dEGFP), destabilised ECFP (dECFP), destabilised EYFP (dEYFP), HcRed, t-HcRed, DsRed, DsRed2, t-dimer2, t-dimer2 (12), mRFPI, pocilloporin, Renilla GFP, Monster GFP, paGFP, Kaede protein or a Phycobiliprotein, or a biologically active variant or fragment of any one thereof. In some embodiments, the acceptor fluorophore is GFP or a variant thereof. In some embodiments, the acceptor fluorophore is GFP².

In other embodiments, the acceptor fluorophore is a non-protein. Examples include, but are not necessarily limited to, an Alexa Fluor dye, Bodipy dye, Cy dye, fluorescein, dansyl, umbelliferone, fluorescent microsphere, luminescent microsphere, fluorescent nanocrystal, Marina Blue, Cascade Blue, Cascade Yellow, Pacific Blue, Oregon Green, Tetramethylrhodamine, Rhodamine, Texas Red, rare earth element chelates, or any combination or derivatives thereof.

In some embodiments, the extracellular bioluminescent sensor molecule may be selected from a BRET¹, BRET², BRET³, BRET⁴, BRET⁵ or BRET⁶ biosensor. In BRET¹ systems, the donor lumiphore is RLuc, acting on native coelenterazine (CLZ) or coelenterazine h (CLZ-h) and the acceptor fluorophore is yellow fluorescent protein (YFP). In BRET² systems, the donor lumiphore is RLuc, acting on bisdeoxycoelenterazine (CLZ 400a) and the acceptor fluorophores is green fluorescent protein or a variant thereof (GFP²). In BRET³ systems, the donor lumiphore is RLuc8, acting on CLZ-h and the acceptor fluorophores is mOrange. In BRET⁴ systems, the donor lumiphore is RLuc8, acting on CLZ-h or coelenterazine v (CLZ-v) and the acceptor fluorophore is TagRFP. In BRET⁵ systems, the donor lumiphore is RLuc8.6, acting on CLZ-h and the acceptor fluorophores is TagRFP. In BRET⁶ systems, the donor lumiphore is RLuc8.6, acting on CLZ-h or CLZ-v and the acceptor fluorophore is TurboFP635.

In exemplary embodiments, the extracellular bioluminescent sensor molecule is a BRET² biosensor. Such biosensors advantageously provide long-Stokes shift bioluminescence resonance energy transfer, with peak donor emission and acceptor emission at 395 nm and 515 nm respectively. Sensitive BRET² biosensors have been developed for detecting a range of analytes, including proteases (e.g. plasmin in milk; Australian patent 2015243093) or sugars (e.g. maltose or lactose; Australian patent 2018315053) or hydrolases (e.g. an esterase or phosphatase; Australian patent application 2018321580).

In some embodiments, an extracellular bioluminescent sensor molecule refers to a BRET biosensor which operates, in the assay composition, in a non-cellular environment or external to any cells which are present. It does not imply that the bioluminescent sensor molecule was produced in an extracellular environment. Indeed, the extracellular bioluminescent sensor molecule may be an engineered protein. For example, the extracellular bioluminescent sensor molecule may be a recombinant protein. Suitable engineered biosensor proteins may be expressed in and purified from E. coli. For example, the biosensor may a recombinant protein expressed in E. coli BL21 (DE3) using the IPTG-inducible pRSET expression system. Biosensors may be translationally fused to a poly-histidine tag used for purification via immobilised metal (e.g. cobalt or nickel) affinity chromatography.

In some embodiments, the extracellular bioluminescent sensor molecule may be selected from a cleavable, allosteric or competition BRET biosensor. BRET biosensors are categorised into these types based on the nature of the interaction of the extracellular analyte with the analyte-sensitive domain. In cleavable BRET biosensors, the analyte-sensitive domain on the linker between the donor lumiphore and acceptor fluorophore comprises a specific peptide target sequence that is cleaved by a protease analyte, delinking the donor lumiphore and acceptor fluorophore and thus causing a change in the BRET ratio. Examples of cleavable BRET biosensors are disclosed in AU2015243093, Australian patent application 2017358067A1, Dacres et al Sensors and Actuators B: Chemical 2019 301 127141 and Dacres et al Analytical Biochemistry 2009 385 (2) 194-202. In some embodiments, the extracellular bioluminescent sensor molecule is thus a protease sensor, e.g. for AprX, Plasmin, Thrombin, Caspase III, and the like.

In allosteric BRET biosensors, the analyte-sensitive domain on the linker between the donor lumiphore and acceptor fluorophore comprises a binding site for a ligand analyte, for example a specific saccharide such as maltose or lactose. The binding site changes shape when bound by the ligand analyte, reorienting the donor lumiphore with respect to the acceptor fluorophore and thus causing a change in the BRET ratio. Examples of allosteric BRET biosensors are disclosed in Australian patent 2018315053, Dacres et al Biosensors & Bioelectronics 2013 41 459-464 and Caron et al Anal Chem 2018 90 (21) 12986-12993. In some embodiments, the extracellular bioluminescent sensor molecule is thus a carbohydrate sensor, e.g. for lactose, maltose, sucrose and the like.

In competition BRET biosensors, a BRET-labelled ligand (labelled with one of the donor lumiphore and acceptor fluorophore) is bound to a BRET-labelled binding protein (labelled with the other of the donor lumiphore and acceptor fluorophore). The BRET-labelled ligand is competitively displaced by an unlabelled ligand analyte, delinking the donor lumiphore and acceptor fluorophore and thus causing a change in BRET ratio. The BRET-labelled binding protein, though initially occupied, is thus the analyte-sensitive domain in such embodiments. An example of a competition BRET biosensor is disclosed in Australian patent application 2020265898.

In assays using competition BRET biosensors, the diagnostic BRET ratio is typically detected when the system reaches an equilibrium between BRET-labelled binding protein bound to analyte and BRET-labelled binding protein bound to BRET-labelled ligand. It will be appreciated that the same equilibrium can be reached by introducing the BRET-labelled binding protein and BRET-labelled ligand as separate species into the assay composition, i.e. as a precursor system comprising a pair of molecules (BRET-labelled binding protein and BRET-labelled ligand) adapted to react (by binding) in solution to form the competition BRET biosensor. Indeed, this may be preferred in some embodiments, since equilibrium may be reached more rapidly when starting from initially unbound species, particularly if the dissociation kinetics are slow.

### Preparing and Incubating the Assay Composition

The methods disclosed herein comprise a step of incubating an assay composition comprising the sample and the biosensor system. The assay composition may optionally comprise other components such as (i) one or more uncaging agents, such as an esterase, for uncaging the caged substrate compound(s) in the assay composition, (ii) buffers, (iii) one or more salts, (iv) one or more metal ions (if required e.g. for metalloprotease assays), (v) one or more carrier proteins, such as fish-skin gelatine, and (vi) one or more cryoprotectants, such as trehalose, sucrose and pullulan. These other components may form part of the solid (biosensor) composition, be added to the sample before combining with the biosensor composition or be added directly to the assay composition.

The assay composition to be incubated may be prepared by any suitable method. In some embodiments, the assay composition is prepared by dispersing a solid composition, comprising at least the bioluminescent sensor molecule or precursor system thereof and typically also various optional components (such as the uncaging agent), in a liquid. The solid composition may be dispersed directly in the liquid sample (either undiluted or diluted) but alternatively it may be dispersed in another aqueous liquid prior to adding the sample. The solid composition, containing sufficient biosensor for a single assay, may be provided in the assay vessel to be used in the incubation and detection steps, thus simplifying the methodology and avoiding or minimising error caused by inaccurate dispensing of reagents into the assay vessel.

The solid composition may be a freeze-dried solid composition, which may suitably be prepared by dispersing or dissolving the extracellular bioluminescent sensor molecule or precursor system thereof and other optional components in an aqueous liquid and freeze-drying the composition by conventional methods. Advantageously, freeze-drying allows the extracellular bioluminescent sensor molecule to be stored over prolonged periods before being reconstituted in the assay composition. The solid composition may be freeze-dried in the assay vessel to be used in the incubation and detection steps.

In some embodiments, a freeze-dried solid composition containing a biosensor system is prepared by charging a suitable assay vessel, e.g. a QSP™ Natural Screw Cap Micro Tubes 520-GRD (ThermoFisher Scientific), with an aqueous solution (e.g. 200 μL) comprising the biosensor system and other components, and freeze-drying the aqueous solution. After the freeze-drying, the assay vessel is sealed and preferably stored in the dark and at room temperature or cooler until required for an analysis. The inventors have demonstrated that such freeze-dried compositions are stable for extended periods, even at elevated storage temperatures, and useable shelf-lives of up to a year or even longer are thus expected. Moreover, the freeze-dried composition can readily be reconstituted in an aqueous liquid sample such as milk, thus facilitating reproducible analyses.

It will be appreciated that the amount of biosensor (e.g. BRET biosensor) present in the solid composition will depend on the specifics of the implementation. It will also be appreciated that the presence and relative amounts of the optional components present in the solid composition will depend on the specifics of the implementation. For example, the solid composition may contain an uncaging agent, but in some embodiments it does not since this component may be endogenous to the sample in which the solid composition is reconstituted, or may be added separately to the assay composition.

The amount of BRET biosensor is typically sufficient for the luminescence intensity under the conditions of the assay to give a low variance in the BRET signal (for example a coefficient of variance of BRET ratio≤1% over a measurement window of 10-20 seconds). Other factors that affect the optimum BRET biosensor concentration include, (i) in the case of a ligand: the affinity of the biosensor for the ligand (e.g. Kd), the concentration range of the ligand expected or needed to be detected in the sample, and the concentration of any competing binding proteins or ligands typically present in the sample, and (ii) in the case of measuring an enzyme activity: the concentration and specific activity of the enzyme typically found or that needs to be measured, the Michaelis constant (Km) and the Kcat of the target enzyme for the biosensor target, the concentrations of any alternate substrates for the enzyme that will compete with the biosensor's target site, and the concentration of other inhibitors typically found in the sample. A suitable amount of BRET biosensor for any particular assay may be determined by routine experimentation, for example by titrating the amount of biosensor down to give a high proportional binding of the biosensor by the analyte, or dissociation in a competition assay, or cleaving of a linker under the conditions of the assay when representative samples are used.

In some embodiments, the concentration of BRET biosensor in the assay composition is between about 10 ng to 2000 ng/ml, about 10 to 1600 ng/ml, about 10 to 1200 ng/ml, about 10 to 1000 ng/ml, 10 to 900 ng/ml, 50 to 800 ng/ml, 50 to 700 ng/ml, 50 ng to 600 ng/L, 50 ng to 500 ng/L, 100 to 900 ng/ml, 200 to 900 ng/ml, 300 to 900 ng/ml, 400 to 900 ng/ml, 500 to 800 ng/ml, or 600 to 800 ng/ml. In some embodiments, the concentration of BRET biosensor in the assay composition is between about 600 to 800 ng/ml. In some embodiments, the concentration of BRET biosensor in the assay composition is about 690 ng/ml.

The assay composition may comprise one or more uncaging agents, such as an esterase, for uncaging the caged substrate compound(s) in the assay composition. Any suitable uncaging agent may be used based on the caged substrate(s) being used in the method. In some embodiments, the uncaging agent is an esterase, for example a porcine esterase. In some embodiments, the concentration of uncaging agent in the assay composition is between about 0.2 and 50 U/mL, 0.2 and 40 U/mL, 0.2 and 20 U/mL, 0.2 and 18 U/mL, 0.2 and 16 U/mL, 0.2 and 14 U/mL, 0.2 and 12 U/mL, 0.2 and 10 U/mL, 1 and 20 U/mL, 1 and 15 U/mL, or 1 and 10 U/mL. In some embodiments, the concentration of uncaging agent in the assay composition is about 1, 5 or 10 U/mL.

The assay composition may comprise one or more buffers. Any suitable buffer may be used. Suitable buffers may include, but are not limited to sodium phosphate, sodium citrate, sodium succinate, histidine (or histidine-HCl), sodium carbonate, CAPS, CAPSO, tris and/or sodium malate. As would be understood by the person skilled in the art the preferred pH (and therefore buffer) will depend on the assay being performed. In some embodiments, the concentration of buffer in the assay composition is sufficient to provide the required buffering capacity. In some embodiments, the concentration of buffer in the assay composition is between about 2 mM and 100 mM, for example 50 mM.

The assay composition comprises one or more cryoprotectants. Suitable cryoprotectants are known to the person skilled in the art. Suitable cryoprotectants may include, but are not limited to sucrose, trehalose, lactose, glycerol, dextrose, raffinose, pullulan, and/or mannitol. In some embodiments, the cryoprotectant is pullulan and/or trehalose. In some embodiments, the cryoprotectant is pullulan. In some embodiments, the cryoprotectant is trehalose. The concentration of the cryoprotectant can be determined by the person skilled in the art. In some embodiments, the concentration of each cryoprotectant in the assay composition is between about 0.2 and 20 mg/mL, for example about 2.5 mg/ml or 5 mg/mL. In some embodiments, the total concentration of cryoprotectant in the assay composition is between about 0.4 and 40 mg/ml, for example about 5 mg/mL.

In some embodiments, the assay composition comprises a carrier protein such as fish gelatine. In some embodiments, the concentration of carrier protein in the assay composition is between about 0.02 and 0.2% (v/v), for example about 0.02, 0.04, 0.06, 0.08, 0.09, 0.1, 0.12, 0.14, 0.16, 0.18 or 0.2% (v/v). In some embodiments, the concentration of carrier protein is about 0.09% (v/v). In some embodiments, the % (v/v) refers to the volume of a stock solution of fish gelatine containing 0.45 g/mL (45% w/v) fish skin gelatine added to the assay composition.

In some embodiments, the assay composition comprises one or more salts, such as sodium chloride. In some embodiments, the concentration of the one or more salts in the assay composition is between about 2 mM and 100 mM, for example 50 mM.

In some embodiments, the assay composition comprises one or more metal ions (if required e.g. for metalloprotease assays or for activity of the enzyme). The metal ion may be provided in the form of a salt. For example, in the case of an assay for AprX, the biosensor system may optionally comprise magnesium chloride and/or calcium chloride. In some embodiments, the concentration of the metal ion in the assay composition is between about 0.2 mM and 20 mM, for example 5 mM.

In some embodiments, the assay composition comprises one or more additional components. Additional components include, but are not limited to, activating agents, inhibitors, enhancers, detergents, reducing agents, oxidising agents and the like. For example, in the case of an assay for a zymogen, the assay composition may optionally comprise an agent that activates the zymogen. For example, in the case of an assay for plasmin which has been disclosed to associate with casein micelles, the assay composition may optionally comprise an agent that dissociates plasmin from the casein micelle. For example, the assay composition may comprise 1 mM aminocaproic acid and 80 mM trisodium acetic acid which helps dissociate the plasmin from the casein micelle. For example, in the case of an assay for plasmin, the assay composition may optionally comprise L-lysine, which supports plasmin activity. In some embodiments, the concentration of L-lysine in the assay composition is between 2 and 100 mM, for example 25 mM. In some embodiment, the assay composition comprises a protease inhibitor, for example a protease inhibitor that inhibits a specific class of proteases (e.g. serine protease inhibitor, cysteine protease inhibitor, metalloprotease inhibitor). Suitable protease inhibitors include PMSF (Phenylmethanesulfonyl fluoride), EDTA, AEBSF (4-(2-Aminoethyl)-benzenesulfonylfluoride hydrochloride), aprotinin, bestatin, E-64, Leupeptin, pepstatin and the like. Protease inhibitors may be included to selectively inhibit the activity of proteases not being assayed for.

The assay composition is incubated for an incubation period sufficient for the extracellular analyte, if present in the sample, to interact with the analyte-sensitive domain. As used herein, the incubation period commences when the extracellular bioluminescent sensor molecule and the sample are first in contact under conditions to allow sustained interaction of the extracellular analyte with the analyte-sensitive domain, and concludes when light is detected from the assay composition to ascertain that interaction. For example, the incubation period may commence when a (freeze-dried) solid composition comprising the bioluminescent sensor molecule is dispersed in a liquid containing (or consisting of) the sample, or when the sample is mixed into a liquid already containing the bioluminescent sensor molecule. The resulting assay composition may then be incubated for the required incubation period before detecting light emitted therefrom. Of course, light may be detected continuously or periodically over an extended detection window, with the relevant incubation period at each detection time progressively increasing.

The assay composition may be incubated at any suitable temperature, for example between 0° C. and 100° C., more typically between 10° C. and 50° C. such as between room temperature (about 22° C.) and 40° C. In some embodiments, the assay composition is incubated at about 37° C. It will be appreciated that higher temperatures may accelerate the interaction of the extracellular analyte and the analyte-sensitive domain, thus necessitating a shorter incubation period. However, higher temperatures may also destabilise one or more components of the assay composition. In some embodiments, the incubation temperature is controlled at a target temperature, or within a narrow (e.g. ±2° C.) temperature range. In some embodiments, incubation with associated temperature control may be conducted in the optoelectronic device which is to be used to detect the light emitted from the assay composition.

The preferred incubation period for any given implementation may depend on the nature of the interaction of the extracellular analyte with the analyte-sensitive domain of the BRET biosensor. Certain cleavable BRET biosensors may require longer incubation times to detect an interaction, for example in the range of 10 to 30 minutes or even longer. Such biosensors may be used to directly measure enzyme activity, where the analyte (a protease) acts on the biosensor. The resultant modification of the biosensor generates a progressive change in BRET signal over an extended time period, corresponding to the kinetics of the enzymatic transformation. Similar considerations would apply to other biosensors comprising an analyte-sensitive domain susceptible to modification by low levels of enzymatic analytes.

On the other hand, certain allosteric and competition BRET biosensors may require only short incubation times, for example in the range of 1 to 10 minutes, such as 2 to 5 minutes. Such biosensors may be used in ligand binding assays where the analyte is a small molecule or macromolecule ligand and is detectable as soon as equilibrium binding to the analyte-sensitive domain (i.e. binding site) has been achieved. This is often rapid, depending on the “on rate” for the equilibrium binding reaction. In principle, the minimum incubation time in such embodiments may be very short, i.e. the detection may in practice be performed immediately after the sample and an extracellular bioluminescent sensor molecule are mixed and sufficient caged substrate compound has been uncaged in the assay composition to energise the donor lumiphore.

However, ligand binding assays are not restricted to short incubation time assays. For example, they may also be useful to measure the accumulation or depletion of a ligand over time in the assay composition by another process, e.g. an enzymatic process, thereby indirectly estimating an enzyme activity. In such cases, longer incubation times may be required since the relevant process being detected is again an enzymatic transformation.

It is a particular advantage of the methods disclosed herein that a range of different incubation periods can be accommodated by adjusting various parameters, including the selection of caged substrate molecule(s) and the amount or activity of the uncaging agent—thus ensuring that the biosensor is suitably energised at the end of the incubation time. In some embodiments, the incubation period is between 1 minute and 60 minutes, or between 1 minute and 30 minutes, or between 5 and 15 minutes, or between 10 minutes and 30 minutes. In some embodiments, the incubation period is 10 minutes or less, for example 2, 4, 6, 8 or 10 minutes.

The methods disclosed herein may be performed as batch assays in a batch assay vessel, and do not require microfluidic or other flow techniques to mediate the incubation of the assay composition or the uncaging of the caged substrate therein. In some embodiments, therefore, the assay composition is incubated without flowing the assay composition through a flow channel, e.g. in a microfluidic device.

### Uncaging a Caged Substrate Compound in the Assay Composition

In embodiments of the methods disclosed herein, at least one caged substrate compound is uncaged in the assay composition during the incubation period to produce an uncaged substrate compound which energises the donor lumiphore when light emitted from the assay composition is to be detected.

As used herein, a caged substrate compound is a protected form of a substrate for the donor lumiphore, in which a reactive functionality of the substrate is reversibly protected with a protecting group. The caged substrate compound may thus be thus unreactive with, or only poorly susceptible to reaction with, the donor lumiphore until it is deprotected to re-form the substrate (also referred to herein as the uncaged substrate compound) in the assay composition. Moreover, the caged substrate compound may be less susceptible to other undesirable reactions, such as autooxidation reactions. Advantageously, the caged substrate compound may be storable in solution for extended periods, making it more readily applicable in various on-the-spot diagnostic test applications. Furthermore, the uncaging of the at least one caged substrate compound may be coordinated with the interaction of analyte with BRET biosensor to ensure that the BRET biosensor is suitably energised at the required detection time.

In some embodiments, only one caged substrate compound is uncaged in the assay composition. In other embodiments, two or more caged substrate compounds are uncaged in the assay composition. In the latter case, the two or more caged substrate compounds are typically protected forms of the same uncaged substrate compound, i.e. they generate the same substrate when uncaged. The different caged forms of that substrate may be selected to uncage at different reaction rates, thus extending the period over which uncaged substrate is available to adequately energise the BRET biosensor. This may advantageously ensure that the BRET analysis is less sensitive to the exact incubation time or temperature profile, or to allow repeated BRET analyses over an extended detection window with reduced risk that the observed BRET ratio is confounded by variable luminosity.

In some embodiments, the caged substrate compound(s) are present in the assay composition from the beginning of the incubation period. This means that the caged substrate compounds(s) are present in the assay composition when the extracellular bioluminescent sensor molecule first contacts the sample or immediately thereafter, for example within one to two minutes as is typical when preparing an assay composition by combining various components sequentially. Thus, for example, the assay composition may be prepared by dispersing a (freeze-dried) solid composition containing the BRET biosensor in a liquid sample, and immediately thereafter adding the caged substrate compound(s), e.g. within two minutes, preferably within one minute or less. The combined liquid assay composition is then incubated for the required incubation period.

Combining all components, including the caged substrate compound(s) and any uncaging agents, in the assay composition from the beginning of the incubation period may be preferred because it simplifies the assay protocol. However, it is not excluded that the caged substrate compound may be added to the assay composition at one or more times during (i.e. partway through) the incubation period. Such an approach may be useful when a very long incubation period (e.g. greater than 30 minutes) is required to detect the interaction of analyte with biosensor. The use of a caged substrate is still advantageous in such scenarios because the energisation of the BRET biosensor can be sustained over longer periods (the concentration of the uncaged substrate compound in solution being governed by both the uncaging and consumption kinetics) and because of the enhanced storability of the caged substrate molecule.

The caged substrate compound(s) may be introduced to the assay composition in solution (referred to herein as a substrate solution). Preferably the solution is a room temperature-stable substrate solution, so that a single solution may be stored and used repeatedly in BRET assays over an extended period of time. As used herein, a room temperature-stable substrate solution is substantially stable for at least one week, preferably at least one month, when stored at about 22-25° C. while excluding air and light exposure. A solution may be considered substantially stable if it provides an emission amplitude of no less than 10% of its original value in a blank (analyte-free) BRET assay with the BRET ratio remaining within ±5% of its original value. In contrast, the inventors have found that uncaged substrate compounds may degrade rapidly in solution, even when rigorously excluding light and air.

The substrate solution may be a non-aqueous solution. The non-aqueous solution may comprise a water-miscible and preferably low-volatility polar aprotic solvent, such as dimethylsulfoxide (DMSO). In some embodiments, the solvent is dry-DSMO, e.g. extra-dry DMSO.

The substrate solution may be introduced into the assay composition by any method suitable for accurate dispensing. In some embodiments, it is dispensed drop-wise into the assay composition, for example with a precision dropper. Dilute DMSO solutions, as an example, can be accurately dispensed as drops of known volume (e.g. 28 μL drops) and the substrate solution may thus be formulated so that the required amount of caged substrate compound(s) is dispensed in one or more such drops. Advantageously, this allows a predetermined amount of caged substrate to be dispensed into an assay composition in a simple-to-implement and sufficiently reproducible manner for on-the-spot diagnostic tests. The substrate solution may be contained and stored in a substrate storage vessel for dispensing into the assay composition. In some embodiments, the substrate storage vessel is configured to dispense an accurate predetermined amount of the substrate solution into the assay composition, e.g. drop-wise.

In some embodiments, the substrate solution is stored for at least one week, or at least one month, or at least six months, before introducing the substrate solution to the assay composition. This advantageously allows the methods disclosed herein to be used in on-the-spot diagnostic tests in environments where it would be unacceptable or undesirable to prepare fresh reagent solutions for each test.

When introduced in a substrate solution, the caged substrate is thus added to the assay as a separate component from the BRET biosensor. Alternatively, the caged substrate may be formulated together with the BRET biosensor, for example as a component of the (freeze-dried) solid composition which is dispersed in the sample. In such embodiments, the solid composition is typically free of an uncaging agent, which may then be added to the assay as a separate component, e.g. in a solution, or as part of the sample itself.

The uncaged substrate compound may be any suitable substrate for the BRET biosensor, i.e. a substrate upon which the donor lumiphore acts to generate (bio) luminescence. Typically, the uncaged substrate compound is a high energy luciferin molecule which is enzymatically oxidised with molecular oxygen by the donor lumiphore. Examples of naturally occurring luciferins in bioluminescent organisms include coelenterazine in the sea pansy (Renilla reniformis), D-luciferin (in conjunction with adenosine triphosphate) in fireflies and other luciferins such as vargulin, furimazine and hispidin. The uncaged substrate compound used in the presently disclosed methods may be such a naturally occurring luciferin molecule or a synthetic analogue thereof.

In some embodiments, the uncaged substrate compound is selected from coelenterazine, vargulin, furimazine and synthetic analogues thereof having the same imidazopyrazinone-based core structure. Such imidazopyrazinone-based substrates are all capable of energising RLuc. In some embodiments, the uncaged substrate compound is a coelenterazine compound. As used herein, a coelenterazine compound refers to native coelenterazine or a native or synthetic analogue thereof having the same imidazopyrazinone core structure. In some embodiments, the uncaged substrate compound thus has a structure according to Formula 1a or Formula 2a:

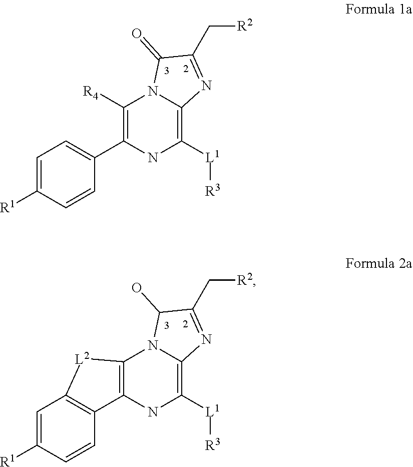

where each R¹ is independently selected from H, OH, OR^{alk} where R^{alk} is C₁-C₆ alkyl, and NH₂, each R² is independently selected from H, Ar, and (CH₂)ₙAr where each Ar is independently a carbocyclic aromatic group optionally substituted with OH, C₁-C₆ alkyl or halogen and n is 1 or 2, each L¹ is independently selected from CH₂ or S, each R³ is independently selected from C₆H₅, CH₂C₆H₅, C₁-C₆ alkyl, and C₁-C₆ cycloalkyl, R⁴ is independently selected from H and CH₃, and L² is independently selected from (CH₂)ₙ where n is 1-3 and CH═CH.

In some embodiments, the uncaged substrate compound has a structure according to Formula 1a, where R¹ is selected from H and OH, R² is selected from C₆H₅ (phenyl) and para-hydroxyphenyl, R³ is C₆H₅, L¹ is CH₂ and R⁴ is H. In some embodiments, the uncaged substrate compound is native coelenterazine, which is a compound according to Formula 1a where R¹ is hydroxy, R² is para-hydroxyphenyl, R³ is phenyl, R⁴ is H and L¹ is CH₂. In other embodiments, the uncaged substrate compound is coelenterazine h, which is a compound according to Formula 1a where R¹ is hydroxy, R² and R³ are phenyl, R⁴ is H and L¹ is CH₂.

In some embodiments, the uncaged substrate compound is bisdeoxycoelenterazine (also known as coelenterazine 400a, Clz400a, Deep Blue C; or 2,8-dibenzyl-6-phenylimidazo[1,2-a]pyrazin-4-ium-3-one). Bisdeoxycoelentarazine is a compound according to Formula 1a where R¹ is H, R² and R³ are each phenyl, R⁴ is Hand L¹ is CH₂. Bisdeoxycoelenterazine has a significantly blue-shifted emission relative to natural coelenterazine, which enables long-Stokes shift resonance energy transfer such as Bioluminescence Resonance Energy Transfer 2 (BRET²), with a greater sensitivity than more red-shifted versions of BRET. Unfortunately, bisdeoxycoelenterazine is highly susceptible to oxidation both as a solid and in solution, and preparations of this compound are therefore inherently unstable under ambient conditions. Bisdeoxycoelenterazine is therefore not suitable as a ready-to-use long-shelf-life reagent.

The first step of the enzymatic oxidation of coelenterazine compounds is believed to involve O₂ binding at the C₂ position of the imidazopyrazinone core structure, adjacent to the carbonyl at the C₃ position (the 3′-carbonyl group). The C₂ and C₃ carbons are labelled in Formula 1a and 2a above. Coelenterazine compounds (including vargulin and furimazine and their synthetic analogues) may thus be caged (i.e. protected against oxidation) by reacting the 3′-carbonyl group with a suitable carbonyl protecting group. In some embodiments, each caged substrate compound used in the method of the present disclosure is a caged coelenterazine compound comprising a carbonyl protecting group at the 3′ position. For example, the 3′-carbonyl may be protected as a 3′-ether functional group, a 3′-ester functional group or a 3′-carbonate ester functional group.

Thus, each caged substrate compound may have a structure according to Formula 1 or Formula 2:

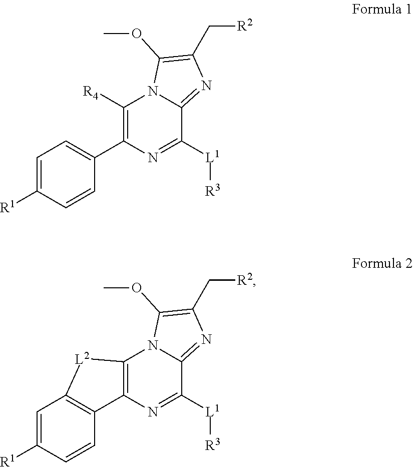

where each R¹ is independently selected from H, OH, OR^{alk} where R^{alk} is C₁-C₆ alkyl, and NH₂, each R² is independently selected from H, Ar, and (CH₂)ₙAr where each Ar is independently a carbocyclic aromatic group optionally substituted with OH, C₁-C₆ alkyl or halogen and n is 1 or 2, each L¹ is independently selected from CH₂ or S, each R³ is independently selected from C₆H₅, CH₂C₆H₅, C₁-C₆ alkyl, and C₁-C₆ cycloalkyl, R⁴ is independently selected from H and CH₃, L² is independently selected from (CH₂)ₙ where n is 1-3 and CH═CH, and each X is independently a carbonyl protecting group.

In some embodiments, each caged substrate compound has a structure according to Formula 1, where R¹ is selected from H and OH, R² is selected from C₆H₅ (phenyl) and para-hydroxyphenyl, R³ is C₆H₅, L¹ is CH₂ and R⁴ is H.

In the compounds of Formula 1 and Formula 2, the 3′ carbonyl group (as was present in the compounds of Formula 1a and Formula 2a respectively) has been protected as a functional group of the form C₃—O—X (where C₃ is the C₃ carbon of the imidazopyrazinone core structure). Similarly, other coelenterazine compounds may be caged by conversion of the 3′ carbonyl group of the imidazopyrazinone core to C₃—O—X, where X is a carbonyl protecting group. In some embodiments, the C₃—O—X functional group is selected from an ether (e.g. C₃—O—CH₂—), an ester (i.e. C₃—O—C[═O]—C) and a carbonate ester (i.e. C₃—O—C[═O]—O—).

In some embodiments, X in the compounds of Formula 1 and Formula 2 has a structure according to Formula 3 (thus forming an ester functional group), Formula 4 (thus forming a carbonate ester functional group) or Formula 5 (thus forming an ether functional group):

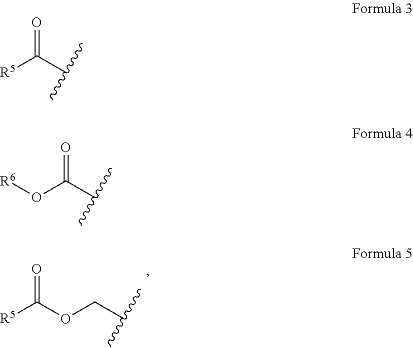

wherein R⁵, R⁶ and R⁷ are each independently optionally substituted C₁-C₁₂ alkyl, C₁-C₁₂ cycloalkyl or C₁-C₁₂ aryl groups, preferably optionally substituted C₁-C₁₂ alkyl or C₁-C₁₂ cycloalkyl groups, and [symbol] represents the point of connection to the 3′-oxygen atom. When the C₁-C₁₂ alkyl, cycloalkyl or aryl groups are substituted, they may be substituted by one or more, for example one to three, groups selected from halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, hydroxy, hydroxyalkyl, alkyloxy, alkyloxyalkyl, alkyloxyaryl, alkyloxyheteroaryl, alkenyloxy, alkynyloxy, cycloalkyloxy, cycloalkenyloxy, heterocycloalkyloxy, heterocycloalkenyloxy, aryloxy, heteroaryloxy, arylalkyl, heteroarylalkyl, arylalkyloxy, amino, alkylamino, acylamino, aminoalkyl, arylamino, sulfonyl, alkylsulfonyl, arylsulfonyl, aminosulfonyl, aminoalkyl, COOH, SH, and acyl. Optionally, such substituents may have no more than 12 carbon atoms.

In some embodiments, R⁵, R⁶ and R⁷ are sterically hindered C₁-C₁₂ alkyl or cycloalkyl groups, for example secondary or tertiary alkyl substituents or bicyclic cycloalkyl substituents, more preferably tertiary substituents such as tertiary butyl groups or adamantyl groups.

Non-limiting examples of suitable caged substrate compounds are disclosed in Jiang et al, Photochem. Photobiol. Sci., 2016, 15, 466 and Yuan et al, Org. Biomol. Chem., 2017, 15, 10238-10244.

The inventors have discovered that caged coelenterazine compounds having an ester, carbonate ester or ether protecting group according to Formula 3 (compound 7 below), Formula 4 (compound 8 below) or Formula 5 (compound 10 below) provide enhanced stability in non-aqueous solutions, thus facilitating their application in on-the-spot diagnostic tests where storage stability of reagents is important. DMSO solutions of each of these compounds were found to be room temperature-stable substrate solutions, each being storable for at least 1 month at room temperature (Example 2). Compound 10 was found to have the greatest stability, and a DMSO solution of this compound can be stored at room temperature for well over a year (Example 1).

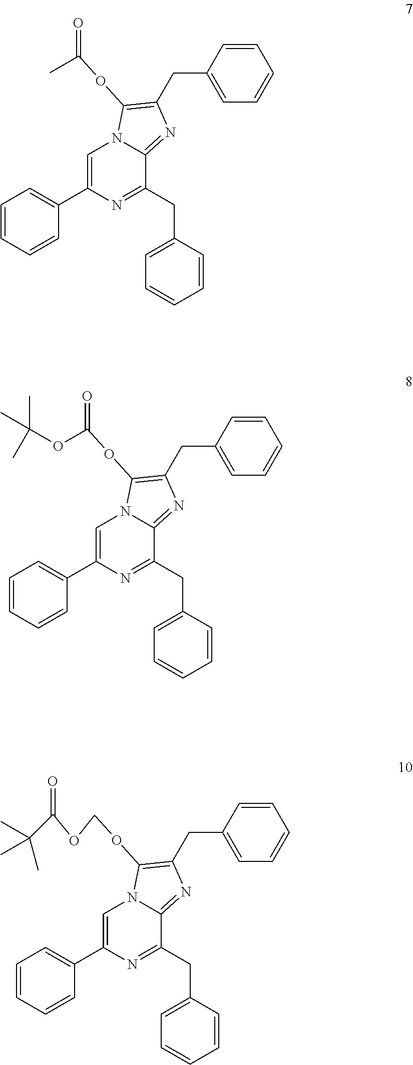 [SMILES: CC(=O)Oc1c(Cc2ccccc2)nc2c(Cc3ccccc3)nc(-c3ccccc3)cn12 ; CC(C)(C)OC(=O)Oc1c(Cc2ccccc2)nc2c(Cc3ccccc3)nc(-c3ccccc3)cn12 ; CC(C)(C)C(=O)OCOc1c(Cc2ccccc2)nc2c(Cc3ccccc3)nc(-c3ccccc3)cn12]

The inventors have also discovered that caged coelenterazine compounds having an ester, carbonate ester or ether protecting group (i.e. compounds 7, 8 and 10 respectively) can be uncaged in situ in an assay composition, with the rate of uncaging dependent both on the nature of the protecting group and the type of uncaging agent that is present (Example 3). In each case, the uncaging generates the corresponding uncaged substrate compound bisdeoxycoelenterazine, thus energising an extracellular BRET² biosensor in the assay composition. Compound 7, with the sterically undemanding acyl protecting group, is rapidly uncaged by either an exogeneous esterase (e.g. porcine liver esterase) or esterases naturally present in a milk sample. Compounds 8 and 10, with their sterically demanding tert-butylcarbonyl (O-Boc) and pivaloyloxymethyl protecting groups respectively, are more slowly uncaged in the presence of the exogeneous esterase, thus energising the extracellular BRET² biosensor over a sustained time period (up to about 30 minutes). By selecting a caged substrate or combination of caged substrates with the appropriate decaging kinetics, when mediated by uncaging agents provided in the assay composition, an extracellular BRET biosensor may be suitably energised for detection of analytes requiring a wide range of different incubation periods, or for continuous or periodic detection of analyte-biosensor interaction over a wide detection window. In some embodiments, the caged substrate is compound 10. In some embodiments, the caged substrate is compound 8. In some embodiments, the caged substrate is compound 7.

The caged substrate compound(s) should be uncaged to provide a suitably strong signal from the BRET biosensor at the time, or over the range of times, when the diagnostic BRET ratio is to be detected. One way to validate this for a given assay protocol, or when developing a new assay protocol to meet this requirement, is to continuously or semi-continuously determine the luminosity associated with the acceptor fluorophore (or the donor lumiphore) during a blank BRET assay, i.e. under conditions representative of a real analysis but without any analyte present that will affect the BRET ratio. The observed luminosity should then be suitably high—for example greater than 50% of its maximum value, or greater than 80% of its maximum value—at the time, or throughout the range of times, when the diagnostic BRET ratio is to be measured in a real analysis.

Therefore, in some embodiments of the methods disclosed herein, the at least one caged substrate compound is uncaged to provide a luminosity associated with the acceptor fluorophore (alternatively with the donor lumiphore), in the absence of the extracellular analyte, that is greater than 50%, or greater than 80% of maximum when detecting the light. The analyte-free luminosity is preferably greater than 50%, or greater than 80% of maximum throughout a detection window of at least 3 minutes, or at least 5 minutes, during which the light emitted from the assay composition is detected one or more times. Such an approach may advantageously minimise the risk that the observed BRET ratio is confounded by a low or changing total luminosity.

While the methods disclosed herein encompass any methods of uncaging the caged substrate compound(s) in situ, the caged substrate compound(s) are most typically uncaged by an uncaging agent or combination of uncaging agents present in the assay composition. Here, it will be appreciated that the uncaging agent is not the BRET biosensor itself.

In some embodiments, the uncaging agent(s) are present in the assay composition from the beginning of the incubation period. Again, this means that the uncaging agent is present in the assay composition when the extracellular bioluminescent sensor molecule first contacts the sample or immediately thereafter, for example within one to two minutes. In some embodiments, the uncaging agent is formulated together with the BRET biosensor. For example, it may be present together with the BRET biosensor in a freeze-dried solid composition suitable for dispersal in the liquid sample. Alternatively, it may be present in the sample or provided as a separate formulation for introduction independently into the assay composition.

While it is commonly advantageous that all components are combined from the beginning of the incubation time, it is not excluded that an uncaging agent may be added to the assay composition at one or more times during (i.e. partway through) the incubation period. Such an approach may be useful to initiate uncaging of the caged substrate(s) when a very long incubation period (e.g. greater than 30 minutes) is required to detect the interaction of analyte with biosensor.

In some embodiments, the uncaging agent is extraneous to the sample. In other embodiments, the uncaging is endogenous to the sample, i.e. it is naturally present in the sample. Some biological matrices, such as milk or other dairy products, contain naturally occurring esterases which can uncage certain caged substrate compounds.

The mechanistic action of the uncaging agent will depend on the chemical form of the protecting group in the caged substrate compound. In some embodiments, the uncaging agent is an esterase or other chemical agent capable of deprotecting a carbonyl protecting group, and in particular a 3′-carbonyl protecting group of a caged coelenterazine compound as disclosed herein. The inventors have found that porcine liver esterase, an industrially available and low cost enzyme, is suitable as the uncaging agent in such embodiments. The inventors have also found that the amount of uncaging agent included in the assay composition may be varied to match the type of biosensor (e.g. allosteric vs cleavable vs competition) and the desired incubation period. In some embodiments, the amount of uncaging agent in the assay composition is between 1 and 100 U/mL, between 1 and 90 U/mL, between 1 and 80 U/mL, between 1 and 70 U/mL, between 1 and 60 U/mL, between 1 and 50 U/mL, between 1 and 40 U/mL, between 1 and 30 U/mL, between 1 and 20 U/mL, or between 1 and 10 U/mL. In some embodiments, the amount of uncaging agent in the assay composition is between 1 and 10 U/mL, or 1, 5 or 10 U/mL.

The present inventors have found that by selecting a caged substrate or combination of caged substrates with the appropriate decaging kinetics, optimally timed light generation for assays of varying durations may be achieved. This has the advantage of energising an extracellular biosensor for detection of analytes requiring a wide range of different incubation periods, or for continuous or periodic detection of analyte-biosensor interaction over a wide detection window. In some embodiments, the caged substrate includes two or more caged substrates of formula 1, two or more caged substrates of formula 2, or at least one caged substrates of formula 1 and at least one caged substrate of formula 2. In some embodiments, the caged substrate is selected from the group consisting of compound 7, compound 8 and compound 10 or a combination thereof. In some embodiments, the caged substrate comprises (i) formula 7 and compound 8, (ii) compound 7 and compound 10, (iii) compound 8 and compound 10 or (iv) compound 7, compound 8 and compound 10. In some embodiments, the caged substrate comprises compound 8 and compound 10. In embodiments where the caged substrate comprises two compounds (e.g. compound 8 and compound 10), the ratio of the two compounds may be between 100:1 and 1:100, or between 50:1 and 1:50, or between 10:1 and 1:10, or between 5:1 and 1:5 or between 3:1 and 1:3, or between 2:1 and 1:3, or between 1:1 and 1:2. In some embodiments, the molar ratio of compound 8 to compound 10 is about 1:1 to 1:5, or 1:1 to 1:4, or 1:1 to 1:3, or 1:1 to 1:2. In some embodiments, the molar ratio of compound 8 to compound 10 is about 1:2. In embodiments were the caged substrate comprises compound 7, 8 and 10, the molar ratio of compound 7:compound 8:compound 10 may be from 1:1:1 to 1:100:100, for example 1:1:1, 1:2:2, 1:6:6, 1:12:12 or 1:60:60.

By providing the substrate in the form of one or more caged substrate compounds formulated in a non-aqueous solution, for in situ uncaging in the assay composition, the inventors have demonstrated that the following balance of properties required for a successful on-the-spot diagnostic BRET-based methodology can be achieved:
Substrate formulation compatible for use in a cell-free biochemical assays, i.e. rather than intracellular assays;
Substrate formulation stable at room temperature storage for several months, in some cases up to a year;
Substrate formulation water-miscible, non-interfering with the BRET biosensor, and not highly flammable or toxic;
Substrate formulations reproducible and provide consistent results across times, operators and locations;
Substrate formulation compatible with a range of sample matrices (e.g. milk) using industrially available uncaging agents, e.g. enzymes;
Substrate provided in situ by an efficient uncaging process conducted under incubation conditions suitable for analyte interaction with the BRET biosensor; and
Substrate formulation tailorable, based on uncaging kinetics of different uncaged substrate compounds, to match assays having different time courses.

### Detecting Light

The methods disclosed herein comprise a step of detecting light emitted from the assay composition after the incubation period. As required in a BRET assay, the detected light includes distinguishable emissions associated with the acceptor fluorophore and with the donor lumiphore. The ratio of luminosity associated with the acceptor fluorophore to luminosity associated with the donor lumiphore (i.e. the BRET ratio) thus indicates an interaction of the extracellular analyte with the analyte-sensitive domain.

The assay composition may be present in an assay vessel during the detection, with the light emitted from the assay composition being detected through the walls of the assay vessel. Suitable assay vessels may thus be transparent at least to the emission wavelengths of the donor and acceptor lumiphores, and at least in regions of the walls configured to allow transmission of light for detection. I In some embodiments, the assay composition is continuously present in the same assay vessel during the incubation period and the detection. In some embodiments, the assay composition is prepared in the same assay vessel.

The light may be detected by any suitable means, including in principle by human eye which is sufficiently sensitive to detect the luminosity and colour change of some BRET assays. More typically, the light is detected by an optoelectronic device capable of distinguishing the emissions associated with the acceptor fluorophore and with the donor lumiphore. The optoelectronic device may be a sophisticated laboratory instrument such as a microplate spectrometer (plate-reader). However, the methods disclosed herein may also be implemented with a simple two-channel optoelectronic device configured to quantify the luminosities of emissions associated with the acceptor fluorophore and with the donor lumiphore, as will be described in greater detail hereafter.

The method may include the step of calculating the BRET ratio of the light emitted from the assay composition, and determining an activity or concentration of the extracellular analyte in the sample from this BRET ratio. This may be done by comparing the observed BRET ratio to a pre-determined calibration curve. As the skilled person will appreciate, the calibration curve may be obtained by measuring the BRET ratio of assays performed with a series of externally defined calibration standards, and fitting a calibration curve to the calibration data, optionally using a non-linear curve fit equation.

### System for Detecting an Extracellular Analyte in a Sample

The present invention further relates to a system for detecting an extracellular analyte in a sample. The system comprises a biosensor system selected from an extracellular bioluminescent sensor molecule or precursor system, as disclosed herein, contained in an assay vessel which is configured to receive the sample for combination with the biosensor system in an assay composition. The system further comprises a room temperature-stable substrate solution comprising at least one caged substrate compound, as disclosed herein, for dispensing into the assay composition in the assay vessel. The substrate solution is contained in a substrate storage vessel. The system further comprises an optoelectronic device configured to detect light emitted from the assay composition through the walls of the assay vessel, including emissions associated with the acceptor fluorophore and with the donor lumiphore. The ratio of luminosity associated with the acceptor fluorophore to luminosity associated with the donor lumiphore indicates an interaction of the extracellular analyte with the analyte-sensitive domain. The at least one caged substrate compound is adapted to uncage in the assay composition in use, thereby producing an uncaged substrate compound which energises the donor lumiphore.

The extracellular bioluminescent sensor molecule (or BRET biosensor) and the room temperature-stable substrate solution, comprising at least one caged substrate compound, may generally be according to any of the embodiments described herein in the context of the methods of the invention. In particular, the extracellular bioluminescent sensor molecule (or precursor system thereof) may be present in a solid composition, optionally together with one or more further components such as uncaging agent. The solid composition may be a freeze-dried solid composition, which may have been prepared by freeze-drying an aqueous precursor composition in the same assay vessel. The room temperature-stable substrate solution may be a non-aqueous solution, preferably comprising a water-miscible and low-volatility polar aprotic solvent such as DMSO. The substrate solution may be formulated at a suitable concentration for dispensing into the assay composition in the assay vessel, for example dropwise.

### Assay Vessel

The assay vessel of the system contains the biosensor system, typically in a solid composition as described herein. The assay vessel is configured to (i) receive a sample for combination with the extracellular bioluminescent sensor molecule in an assay composition, and (ii) to allow light emitted from the assay composition to be detected through the walls of the assay vessel. As used herein, the walls of an assay vessel through which light is transmitted are not necessarily the side walls, but could include any portion of the enclosure such as the bottom or cap/lid.

The assay vessel typically contains sufficient extracellular bioluminescent sensor molecule (or precursor) for a single assay only, the amount of which will depend on the specifics of the implementation. In one exemplary embodiment disclosed herein, for analysis of AprX in a 1 ml sample of milk, the assay vessel comprises a freeze-dried solid composition containing about 690 ng of a BRET² biosensor, together with other components including porcine liver esterase. In another exemplary embodiment disclosed herein, for analysis of plasmin or plasminogen in a 1 ml sample of milk, the assay vessel comprises a freeze-dried solid composition containing about 690 ng of a BRET² biosensor, together with other components including porcine liver esterase. In another exemplary embodiment disclosed herein, for analysis of lactose in a 1 ml sample of milk, the assay vessel comprises a freeze-dried solid composition containing about 500 ng of a BRET² biosensor, together with other components including porcine liver esterase.

The assay vessel may be in the form of a sealable tube, e.g. fitted with a screw cap, and having a suitable capacity to receive the sample, for example at least 1 ml of sample. The tubes may be transparent, at least to light emitted by the BRET biosensor, thus allowing the light to be detected through the side walls. In some embodiments, the tubes are precision moulded from an optically transparent thermoplastic such as polypropylene and have no markings or colouring on the tube walls. One example of suitable tubes is sealable Thermo Scientific™ QSP™ Natural Screw Cap Micro Tubes 520-GRD tubes, which have a diameter of 1 cm and a height (without cap) of 4.4 cm. Whilst assay vessels made with different materials and having different shapes or dimensions may be used, in preferred embodiments they can be sealed, allow efficient transmission of visible light through the walls, are optically identical under rotation and closely match the dimensions of the tube-holder in the optoelectronic device.

The system may further comprise a light-tight outer container, preferably containing a desiccant, in which one or more assay vessels containing the BRET biosensor may be stored at room temperature or cooler until required for use.

### Solid Composition and Precursor Composition

The biosensors used in the methods and other aspects and embodiments described herein are typically provided in a solid composition. Accordingly, there is also provided a solid composition that is suitable for use in the embodiments and aspects described herein. The solid composition comprises the biosensor (e.g. BRET biosensor). The solid composition may optionally comprise other components such as (i) one or more uncaging agents, such as an esterase, for uncaging the caged substrate compound(s) in the assay composition, (ii) buffers, (iii) one or more salts, (iv) one or more metal ions (if required e.g. for metalloprotease assays), (v) one or more carrier proteins, such as fish-skin gelatine, and (vi) one or more cryoprotectants, such as trehalose, sucrose and pullulan.

The solid composition typically contains sufficient extracellular bioluminescent sensor molecule (or precursor) for a single assay only, the amount of which will depend on the specifics of the implementation. In some embodiments, the assay vessel comprises a solid composition containing about 10 ng to 1000 ng of biosensor, or about 10 ng to 900 ng, 10 ng to 800 ng, 10 ng to 700 ng, 50 ng to 1000 ng, 100 ng to 1000 ng, 200 ng to 1000 ng, 300 ng to 1000 ng, 400 ng to 1000 ng, 500 ng to 1000 ng, or 600 ng to 1000 ng. In some embodiments, the assay vessel comprises a freeze-dried solid composition containing about 400 to 700 ng of biosensor. In some embodiments, the assay vessel comprises a freeze-dried solid composition containing about 690 ng of biosensor. In some embodiments, the assay vessel comprises a freeze-dried solid composition containing about 500 ng of biosensor. In one exemplary embodiment disclosed herein, for analysis of AprX in a 1 ml sample of milk, the solid composition comprises about 690 ng of a BRET² biosensor, together with other components including porcine liver esterase. In another exemplary embodiment disclosed herein, for analysis of plasmin or plasminogen in a 1 ml sample of milk, the solid composition comprises about 690 ng of a BRET² biosensor, together with other components including porcine liver esterase. In another exemplary embodiment disclosed herein, for analysis of lactose in a 1 ml sample of milk, the solid composition comprises about 500 ng of a BRET² biosensor, together with other components including porcine liver esterase.

The solid composition may comprise one or more uncaging agents as defined herein. In some embodiments, the amount of uncaging agent (e.g. esterase) in the solid composition is between about 0.2 and 20 Units, 0.2 and 18 Units, 0.2 and 16 Units, 0.2 and 14 Units, 0.2 and 12 Units, 0.2 and 10 Units, 1 and 20 Units, 1 and 10 Units, or 1 and 5 Units. In some embodiments, the concentration of uncaging agent is about 1, 5 or 10 Units.

The solid composition may comprise one or more buffers as defined herein. In some embodiments, the amount of buffer in the solid composition is between about 2 μmoles and 100 μmoles, for example 50 μmoles.

The solid composition may comprise one or more cryoprotectants as defined herein. In some embodiments, the amount of each cryoprotectant in the solid composition is between about 0.2 and 20 mg, for example 2.5 mg or 5 mg. In some embodiments, the total amount of cryoprotectant in the solid composition prior to freezing is between about 0.4 and 40 mg, for example 5 mg.

In some embodiments, the solid composition may comprise a carrier protein such as fish gelatine in a suitable amount.

In some embodiments, the solid composition may comprise one or more salts, such as sodium chloride. In some embodiments, the amount of the one or more salts in the solid composition is between about 2 μmoles and 100 μmoles, for example 50 mM μmoles.

In some embodiments, the solid composition comprises one or more metal ions (if required e.g. for metalloprotease assays or for activity of the enzyme). In some embodiments, the amount of the metal ion in the solid composition is between about 0.2 μmoles and 20 μmoles, for example 5 μmoles.

In some embodiments, the solid comprises one or more additional components as described herein. For example, in the case of an assay for a zymogen, the solid composition may optionally comprise an agent that activates the zymogen. For example, in the case of an assay for plasmin which has been disclosed to associate with casein micelles, the solid composition may optionally comprise an agent that dissociates plasmin from the casein micelle. For example, the solid composition may comprise 1 μmole aminocaproic acid and 80 μmole trisodium acetic acid which helps dissociate the plasmin from the casein micelle. For example, in the case of an assay for plasmin, the solid composition may optionally comprise L-lysine, which supports plasmin activity. In some embodiments, the amount of L-lysine in the solid composition is between 2 μmole and 100 μmole, for example 25 μmole.

In some embodiments, the solid composition is prepared by charging a suitable assay vessel with an aqueous solution of the composition (referred to herein as the precursor composition). The volume of aqueous solution added to the assay vessel may vary, but will typically provide extracellular bioluminescent sensor molecule (or precursor) for a single assay only, the amount of which will depend on the specifics of the implementation. In some embodiments, the solid composition is prepared by charging a suitable assay vessel with 200 μL of the precursor composition. Unless indicated otherwise, the following paragraphs describe the concentration of certain components in the aqueous solution of the composition prior to freeze or drying (i.e. the concentration in the precursor composition).

In some embodiments, the concentration of BRET biosensor in the composition prior to freezing is between about 50 ng to 10000 ng/ml, or about 50 to 8000 ng/ml, about 50 to 6000 ng/mL, about 50 to 5000 ng/mL, 50 to 4500 ng/mL, 50 to 4000 ng/ml, 50 to 3500 ng/ml, 50 ng to 3000 ng/L, 500 to 4000 ng/ml, 1000 to 4000 ng/ml, 1500 to 4000 ng/ml, 2000 4000 ng/ml, 2500 to 4000 ng/ml, or 3000 4000 ng/ml. In some embodiments, the concentration of BRET biosensor is between about 3000 to 4000 ng/ml. In some embodiments, the concentration of BRET biosensor is about 3450 ng/ml. In some embodiments, the concentration of BRET biosensor is about 2500 ng/ml.

The precursor composition may comprise one or more uncaging agents as defined herein. In some embodiments, the concentration of uncaging agent (e.g. esterase) in the composition prior to freezing is between about 1 and 100 U/mL, 1 and 90 U/mL, 1 and 80 U/mL, 1 and 70 U/mL, 1 and 60 U/mL, 1 and 50 U/mL, 5 and 100 U/mL, 5 and 50 U/mL, or 5 and 25 U/mL. In some embodiments, the concentration of uncaging agent is about 5, 25 or 50 U/mL.

The precursor composition may comprise one or more buffers as defined herein. In some embodiments, the concentration of buffer in the composition prior to freezing is between about 10 mM and 500 mM, for example 250 mM.

The precursor composition may comprise one or more cryoprotectants as defined herein. In some embodiments, the concentration of each cryoprotectant in the composition prior to freezing is between about 1 and 100 mg/mL, for example 12.5 mg/mL or 25 mg/mL. In some embodiments, the total concentration of cryoprotectant in the composition prior to freezing is between about 2 and 200 mg/mL, for example 25 mg/mL.

In some embodiments, the precursor composition may comprise a carrier protein such as fish gelatine. In some embodiments, the concentration of carrier protein in the composition prior to freezing is between about 0.1 and 1% (v/v), for example about 0.1, 0.2, 0.3, 0.4, 0.45, 0.5, 0.6, 0.7, 0.8, 0.9 or 1% (v/v). In some embodiments, the concentration of carrier protein is about 0.45% (v/v). In some embodiments, the % (v/v) refers to the volume of a stock solution of fish gelatine containing 0.45 g/mL (45% w/v) fish skin gelatine added to the precursor composition.

In some embodiments, the precursor composition may comprise one or more salts, such as sodium chloride. In some embodiments, the concentration of the one or more salts in the composition prior to freezing is between about 10 mM and 500 mM, for example 250 mM.

In some embodiments, the precursor composition comprises one or more metal ions (if required e.g. for metalloprotease assays or for activity of the enzyme) . . . . In some embodiments, the concentration of the metal ion in the composition prior to freezing is between about 1 mM and 100 mM, for example 25 mM.

In some embodiments, the precursor comprises one or more additional components. Additional components include, but are not limited to, activating agents, inhibitors, detergents, reducing agents, oxidising agents and the like. For example, in the case of an assay for a zymogen, the assay composition may optionally comprise an agent that activates the zymogen. For example, in the case of an assay for plasmin which has been disclosed to associate with casein micelles, the precursor composition may optionally comprise an agent that dissociates plasmin from the casein micelle. For example, the precursor composition may comprise 5 mM aminocaproic acid and 400 mM trisodium acetic acid which helps dissociate the plasmin from the casein micelle. For example, in the case of an assay for plasmin, the precursor composition may optionally comprise L-lysine, which supports plasmin activity. In some embodiments, the concentration of L-lysine in the composition prior to freezing is between 10 and 500 mM, for example 125 mM.

In one exemplary embodiment, a freeze-dried solid composition containing a BRET² biosensor for detection of AprX in milk is prepared by charging a suitable assay vessel, e.g. a QSP™ Natural Screw Cap Micro Tubes 520-GRD (ThermoFisher Scientific), with an aqueous solution (200 μL) of the following composition, and freeze-drying the aqueous solution:

| | |
|---|---|
| Precursor composition | Assay composition |

| | | | | |
|---|---|---|---|---|
| AprX biosensor | 3,450 | ng/ml | 690 | ng/ml |
| Tris (pH 8.4) | 250 | mM | 50 | mM |
| NaCl | 250 | mM | 50 | mM |
| MgCl₂•6H₂O | 25 | mM | 5 | mM |
| CaCl₂•2H₂O | 25 | mM | 5 | mM |
| fish skin gelatine* | 0.45% | (v/v) | 0.09% | (v/v) |
| Trehalose | 12.5 | mg/mL | 2.5 | mg/mL |
| Pullulan | 12.5 | mg/ml | 2.5 | mg/mL |

| | | |
|---|---|---|
| porcine esterase | 5 U/mL or 25 U/mL | 1 U/mL or 5 U/mL |

*The stock solution contains 45% (w/v) (0.45 g/mL) fish skin gelatine.

In one exemplary embodiment, the above formulation with 25 U/mL esterase (final concentration of 5 U/mL) may also be used for the detection of Subtilisin (Alcalase).

In one exemplary embodiment, a freeze-dried solid composition containing a BRET² biosensor for detection of plasmin in milk is prepared by charging a suitable assay vessel, with an aqueous solution (e.g. 200 μL) of the following composition, and freeze-drying the aqueous solution:

| | Precursor composition | | Assay composition | |
|---|---|---|---|---|
| Plasmin BRET² biosensor | 3,450 | ng/ml | 690 | ng/ml |
| Tris (pH 8.4) buffer | 250 | mM | 50 | mM |
| NaCl | 250 | mM | 50 | mM |
| L-Lysine | 125 | mM | 25 | mM |
| Aminocaproic acid | 5 | mM | 1 | mM |
| Trisodium acetic acid | 400 | mM | 80 | mM |
| fish skin gelatine* | 0.45% | (v/v) | 0.09% | (v/v) |
| Trehalose | 12.5 | mg/ml | 2.5 | mg/mL |
| Pullulan | 12.5 | mg/mL | 2.5 | mg/mL |
| porcine esterase | 25 | U/mL | 5 | U/mL |

*The stock solution contains 45% (w/v) (0.45 g/mL) fish skin gelatine.

In one exemplary embodiment, a freeze-dried solid composition containing a BRET² biosensor for detection of plasmin in milk is prepared by charging a suitable assay vessel, with an aqueous solution (e.g. 200 μL) of the following composition, and freeze-drying the aqueous solution:

| | Precursor composition | | Assay composition | |
|---|---|---|---|---|
| Plasmin BRET² biosensor | 3,450 | ng/ml | 690 | ng/ml |
| Tris (pH 8.4) buffer | 250 | mM | 50 | mM |
| NaCl | 250 | mM | 50 | mM |
| L-Lysine | 125 | mM | 25 | mM |
| Aminocaproic acid | — | | — | |
| Trisodium acetic acid | — | | — | |
| fish skin gelatine * | 0.45% | (v/v) | 0.09% | (v/v) |
| Trehalose | 12.5 | mg/mL | 2.5 | mg/mL |
| Pullulan | 12.5 | mg/mL | 2.5 | mg/mL |
| porcine esterase | 5 | U/mL | 1 | U/mL |

* The stock solution contains 45% (w/v) (0.45 g/mL) fish skin gelatine.

In one exemplary embodiment, a freeze-dried solid composition containing a BRET² biosensor for detection of plasminogen in milk is prepared by charging a suitable assay vessel, with an aqueous solution (200 μL) of the following composition, and freeze-drying the aqueous solution:

| | Precursor composition | | Assay composition | |
|---|---|---|---|---|
| Plasmin biosensor | 3,450 | ng/ml | 690 | ng/ml |
| Tris (pH 8.4) buffer | 250 | mM | 50 | mM |
| NaCl | 250 | mM | 50 | mM |
| L-lysine | 125 | mM | 25 | mM |
| fish skin gelatine* | 0.45% | (v/v) | 0.09% | (v/v) |
| Trehalose | 12.5 | mg/ml | 2.5 | mg/mL |
| Pullulan | 12.5 | mg/mL | 2.5 | mg/mL |
| porcine esterase | 5 | U/mL | 1 | U/mL |
| human urokinase | 1000 | U/mL | 200 | U/mL |

*The stock solution contains 45% (w/v) (0.45 g/mL) fish skin gelatine.

In one exemplary embodiment, a freeze-dried solid composition containing a BRET² biosensor for detection of lactose in milk is prepared by charging a suitable assay vessel, with an aqueous solution (200 μL) of the following composition, and freeze-drying the aqueous solution:

| | | | Precursor | | Assay | |
|---|---|---|---|---|---|---|
| | Range | | composition | | composition | |
| Lactose biosensor | 25-250 | nM | 25 | nM | 5 | nM |
| Tris (pH 7.5) | 0-250 | mM | 50 | mM | 10 | mM |
| NaCl | 0-250 | mM | 50 | mM | 10 | mM |
| fish skin gelatine * | 0-0.45% | (v/v) | 0.45% | (v/v) | 0.09% | (v/v) |
| Trehalose | 0-25 | mg/ml | 0 | | 0 | |
| Pullulan | 0-25 | mg/ml | 25 | mg/mL | 5 | mg/mL |
| porcine esterase | 50 | U/mL | 50 | U/mL | 10 | U/mL |

* The stock solution contains 45% (w/v) (0.45 g/mL) fish skin gelatine.

In each of the exemplary embodiments, the assay composition is formed by dispersing the freeze-dried composition in the sample being analysed (1 mL).

### Substrate Storage Vessel

The substrate storage vessel of the system contains the room temperature-stable substrate solution. Thus, the substrate solution can be stored until required for use, when it is dispensed into the assay composition in the assay vessel. The substrate storage vessel may be configured to store the substrate solution for at least one week, or at least one month, or at least six months. Thus, the substrate storage vessel is sealable and preferably light-tight (or stored in a light-tight container), thereby minimising air and light exposure during storage. The substrate storage vessel may contain sufficient room temperature-stable substrate solution for multiple assays, for example at least ten. Thus, the caged substrate compound is conveniently provided for periodic on-the-spot diagnostic BRET tests without the need to prepare a fresh formulation each time.

In some embodiments, the substrate storage vessel is configured to dispense an aliquot of the substrate solution into the assay vessel, for example by dispensing the substrate solution drop-wise. The substrate storage vessel may thus be fitted with a precision dropper, capable of accurately and repeatedly dispensing drops of known volume. An example of a suitable substrate storage vessel for this purpose is a Nalgene™ dropper bottle with control dispensing tip, available from ThermoFisher Scientific. Dilute DMSO solutions, as an example, can be accurately dispensed as 28 μL drops from such vessels. The substrate solution may thus be formulated such that the required amount of caged substrate compound(s) for each assay is dispensed in one or more drops. In some embodiments, each drop of substrate solution contains between 1 and 100 mM, between 1 and 90 mM, between 1 and 80 mM, between 1 and 70 mM, between 1 and 60 mM, between 1 and 50 mM, between 1 and 45 mM, between 1 and 40 mM, between 1 and 30 mM, between 1 and 20 mM, between 1 and 10 mM, or between 1 and 5 mM, such as 43 mM, 12 mM, 6 mM or 4.3 mM. In some embodiments, the concentration of each caged substrate in the substrate solution is between about 1 and 50 mM, 1 and 40 mM, 1 and 30 mM, 1 and 20 mM, 1 and 15 mM, 1 and 12 mM, 1 and 10 mM, 1 and 9 mM, 1 and 8 mM, 1 and 7 mM, 1 and 6 mM, or 1 and 5 mM. In some examples, the concentration of the caged substrate compound(s) is about 43 mM of caged substrate compound(s). In some examples, the concentration of the caged substrate compound(s) is about 12 mM of caged substrate compound(s). In some examples, the concentration of the caged substrate compound(s) is about 6 mM of caged substrate compound(s). In some examples, the concentration of the caged substrate compound(s) is about 4.3 mM of caged substrate compound(s).

### Optoelectronic Device

The optoelectronic device of the system is configured to detect light emitted from the assay composition through the walls of the assay vessel, including emissions associated with the acceptor fluorophore and with the donor lumiphore. To avoid interference with the BRET signal, the optoelectronic device is generally configured to exclude all external light when detecting light emitted from the assay composition. In preferred embodiments, the optoelectronic device is configured to receive the assay vessel and to incubate the assay composition contained therein for the required incubation period. The optoelectronic device may thus be configured to incubate the assay composition at a predetermined incubation temperature or within a narrow incubation temperature range (e.g. +2° C.). Advantageously, this may allow the incubation and light detection steps of the method disclosed herein to be performed in a single device and preferably without manual operator intervention after initiation.

The optoelectronic device of the system includes one or more light detectors, for example two separate light detectors tuned to measure the wavelength bands associated with the donor lumiphore and the acceptor fluorophore. Each light detector may include a micro-photon multiplier tube (for example a Hamamatsu H12406 Micro-PMT photon counting head) fitted with a band pass filter adapted to selectively transmit light wavelengths associated only with the donor lumiphore or the acceptor fluorophore. For a BRET² system, examples of suitable bandpass interference filters are a 10 mm diameter Chroma 515 nm/30 nm (green) and 10 mm diameter Chroma 410 nm/80 nm (blue) filters. Conveniently such filters may be exchanged when necessary to sample other wavelengths, e.g. if using a different BRET system.

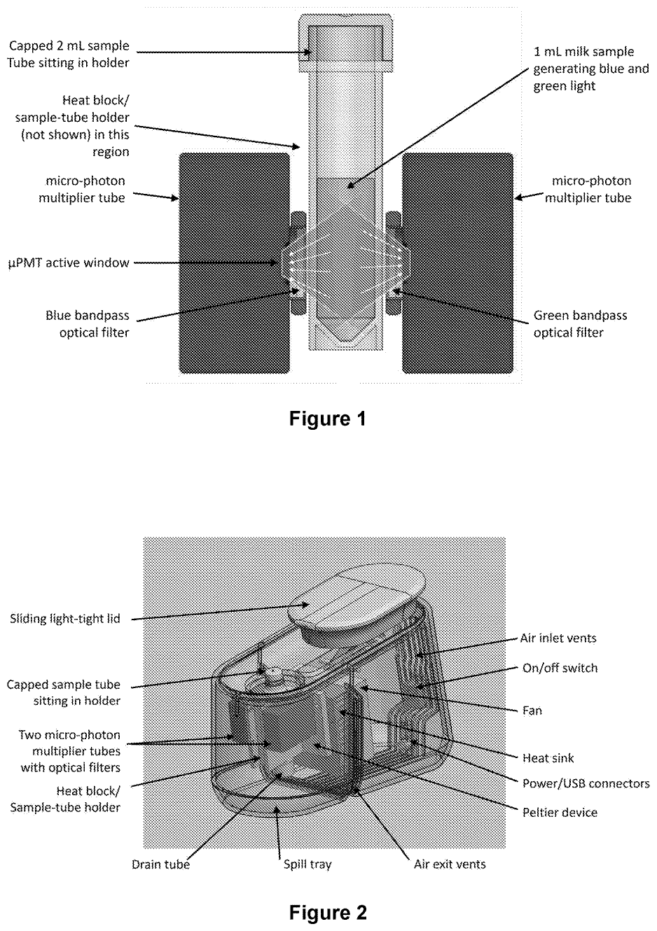

Depicted in FIG. 1 and FIG. 2 is an optoelectronic device according to some embodiments, in particular for deployment in on-the-spot diagnostic testing in production and other non-laboratory environments. As seen in FIG. 1, an assay vessel (e.g. a capped 2 ml transparent polypropylene sample tube) containing a light-emitting assay composition (e.g. 1 ml milk sample+BRET² biosensor+caged substrate+uncaging agent) is received in the optoelectronic device in a sample holder which positions the assay composition between two light detectors. The assay composition may be incubated in the device for the required incubation time before the light is detected. The two light detectors include micro-photon multiplier tubes fitted with either a blue bandpass optical filter (to measure the emissions associated with the BRET² donor lumiphore) or a green bandpass optical filter (to measure the emissions associated with the BRET² acceptor fluorophore). In use, light is transmitted from the assay composition, through the walls of the sample tube, and detected by the two light detectors. The emitted luminosities associated with the acceptor fluorophore and the donor lumiphore are thus quantified, with the BRET² ratio providing an indication of any interaction of an extracellular analyte of interest (e.g. AprX protease) with the BRET² biosensor.

FIG. 2 depicts the complete optoelectronic device including the portion seen in FIG. 1. The optoelectronic device includes a sample holder to receive the sample tube, a sliding light-tight lid to exclude extraneous light during light detection, a Peltier device (heater/cooler) and associated heat sinks, fan, temperature monitor and control electronics to control the incubation temperature, and associated physical and electronic equipment, for example a drain tube and spill tray (in case of sample leaking), and air inlet vents. The optoelectronic device may comprise a USB connection as depicted or other interface for wired or wireless connection to a computing device.

### Software and Quantification

The system disclosed herein may include a computing device which comprises one or more tangible non-transitory computer-readable media having computer-executable instructions for performing computer-implemented methods to control the operation of the optoelectronic device and to process the data received therefrom. Typically, the computer implemented methods are automatically executed on a computer processor, either provided with or separately to the optoelectronic device, e.g. in a desktop computer, tablet or smartphone. The computer processor may include a software application installed thereon for executing one or more of the steps of the computer implemented methods. In alternative embodiments, the software application may be a cloud-based application accessible via a network such as the internet. In some embodiments, the software application may be accessible remotely via a local network.

For each particular sample type, extracellular analyte and BRET biosensor combination, the optoelectronic device may be calibrated by measuring the BRET results of assays performed with a series of externally defined calibration standards. A calibration curve may then then fitted to the calibration data, optionally using a non-linear curve fit equation. A software application installed on the computer processor, such as the GraphPad Prism software, may be used to do this. The curve fit equation and parameters are entered into the software and used by the software to calculate the analyte concentration (e.g. a ligand concentration) or activity (e.g. a protease activity) for each assay of a sample containing an unknown amount of the analyte. This result is recorded and presented to the user, who interprets the value in terms of their actions. In some embodiments, the software is programmed to instruct the user as to what to do in the light of any particular result, e.g. accept a shipment, retest a shipment, reject a shipment or seek further advice.

The operation of the optoelectronic device may also be controlled by a software application on the computer processor, preferably through a user-friendly graphical interface which allows the user to input the sample and operator details, the assay format (time and temperature) etc. The software may capture and record the data from the optoelectronic device during the assay, including for example the temperature and the photon counts in the blue and green channels sampled, for example, every 0.5 seconds during the assay. The software may also calculate and present the BRET ratio in real time.

### Workflow

The system disclosed herein may be used to perform a method of detecting an extracellular analyte in a sample. Advantageously, the method may be implemented as a simple workflow suitable for implementation in a production or other non-laboratory environment.

In some embodiments, the workflow comprises the following steps:
(a) Collect a sample for analysis in a suitable sample collection vessel. In some exemplary embodiments, the sample may be raw or processed milk, for example collected in a specimen jar, beaker, or cup from the sampling port of a tanker, buffer tank, silo or aseptic tank or from a finished milk container.
(b) Select the type of assay to be performed on a computing device connected to the optoelectronic device, by inputting the relevant parameters or selecting a pre-programmed assay. In the exemplary embodiments, the assay type may be an AprX standard sensitivity assay, designed to determine the activity of AprX in milk using a AprX-sensitive BRET² biosensor in combination with one or more caged coelenterazine compounds and one or more uncaging agents, as disclosed herein. Details of the operator and the sample may also be inputted.
(c) Dispense the required volume of sample into an assay vessel already containing the biosensor and optional components such as uncaging agent. Optionally, the sample and solid composition may be agitated to disperse the solid composition in the liquid sample. In the exemplary embodiments, 1 ml of the milk sample is dispensed into a 2 ml assay tube with an auto-dispenser or pipette. The assay tube contains a freeze-dried solid comprising the required amount of AprX-sensitive BRET² biosensor (e.g. 690 ng), a predetermined amount of porcine liver esterase and other components as disclosed herein.
(d) Immediately thereafter (e.g. within one minute) dispense an aliquot of the substrate solution containing one or more caged substrate compounds from a substrate storage vessel into the assay composition, and mix thoroughly to provide a well-mixed assay composition with the solid composition dispersed therein. In the exemplary embodiments, the assay tube is uncapped and one drop (28 μL) of substrate solution comprising 4.3 mM of caged coelenterazine compound(s) is dispensed into the assay composition from a dropper bottle equipped with precision dropper. The assay tube is capped and inverted 10 or 20 times to mix the assay composition.
(e) Insert the assay vessel into the optoelectronic device and initiate the specified assay on the computing device connected to the optoelectronic device. The computing device thus commands the optoelectronic device to incubate the assay composition for the required incubation time, at a specified incubation temperature, and then to detect light emitted from the assay composition. In the exemplary embodiments, the assay tube is inserted into the sample holder of the optoelectronic device depicted in FIGS. 1 and 2, the sliding lid is closed to exclude extraneous light and the AprX standard sensitivity assay is initiated using the software application which controls the device. The optoelectronic device thus automatically incubates the assay composition at the required incubation temperature (e.g. 37° C.) for the required incubation period (e.g. 10 or 15 minutes), and detects the BRET ratio at the end of the incubation period.
(f) Receive the results presented on the computing device after the assay is complete. Typically, the result will be presented as an activity or concentration of the extracellular analyte of interest, derived by comparison of the observed BRET ratio against a pre-established calibration curve.

In some embodiments, all or part of the workflow may be automated. For example, steps (a), (c) and/or (d) may be performed using a preparation station as described herein. Automation, of at least part of the workflow, is thought to enable operators with minimal training and less developed fine-motor skills to generate highly reproducible results. In some embodiments, the preparation station is configured to extract a defined volume of the sample from a suitable sample collection vessel and transfer the sample to an opened reagent tube (e.g. an assay vessel (e.g. a capped 2 ml transparent polypropylene sample tube) containing a light-emitting assay composition (e.g. 1 ml milk sample+BRET² biosensor+caged substrate+uncaging agent)). The preparation station may also be configured to facilitate the placement of the opened reagent tube and allow for the movement of the tube among different (e.g. two or more, or three) positions. In some embodiments, a rail system (e.g. a linear rail system) is used to facilitate positioning and movement of the opened reagent tube. The preparation station may also be configured to accommodate a removable dropper bottle holder containing a dropper bottle filled with substrate solution. In some embodiments, the preparation station comprises a cavity (e.g. a cylindrical cavity) capable of accommodating the removable dropper bottle holder. The preparation station may be configured such once the dropper bottle holder is inserted in the cavity, the cavity becomes airtight. In some embodiments, the cavity may also be shaped and/or include means for fitting a desiccant pad. In some embodiments, the cavity is positioned to enable the dispensing of a drop of substrate solution into the reagent tube. In some embodiments, the cavity may be positioned over the tube located within the rail system, enabling the dispensing of a drop of substrate solution into the reagent tube.

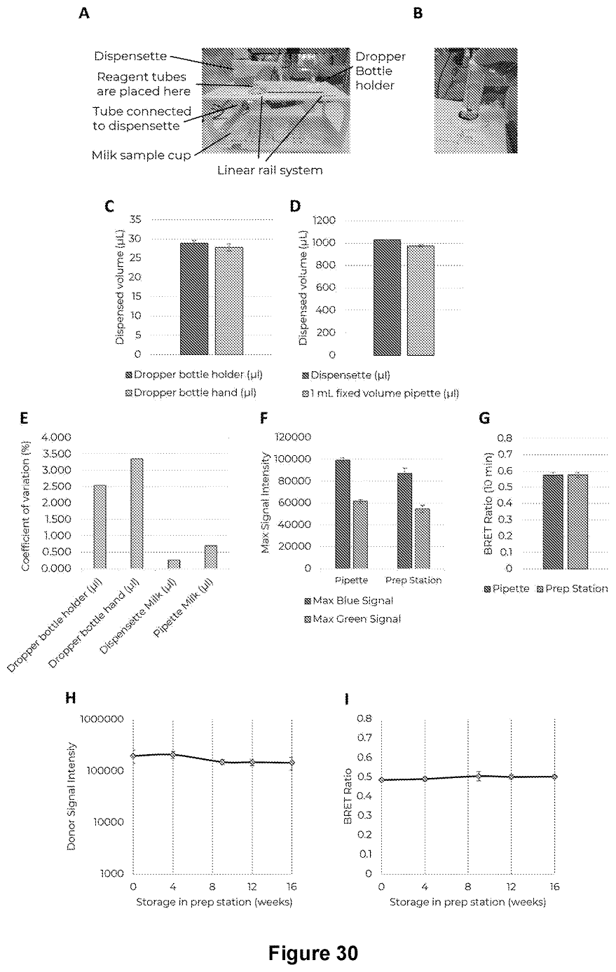

In an exemplified embodiment, the preparation station, as shown in FIG. 29A, comprises of the following components:
a dispensette dispenser designed to extract samples from a sample collection vessel (e.g. a sample cup).
A linear rail system that facilitates the placement of an opened reagent tube and allows for the movement of the tube among three different positions.
A cylindrical cavity that accommodates a removable dropper bottle holder containing a dropper bottle filled with substrate solution. The bottle holder can be positioned over the tube located within the rail system, enabling the dispensing of a drop of substrate solution into the reagent tube. Once the dropper bottle holder is inserted, the cavity becomes airtight and includes provisions for fitting a desiccant pad.

In some embodiments, the preparation station may also be used to dilute samples, prior to analysis with a biosensor. This may be important, for example, when using a high sensitivity BRET-based lactose sensor to measure the lactose concentration in milk that has not been treated with lactase enzyme or where the hydrolysis is at an early stage.

Advantageously, the preparation station offers a simplified assay preparation workflow for operators with minimal training without compromising assay performance. The storage stability of the substrate solution within the preparation station may further improve the ease-of-use of the overall system.

### BRET Reagent System, Solid Biosensor Composition, Substrate Dispensing System and Substrate Composition

The present invention further relates to a BRET reagent system. The BRET reagent system comprises two separate formulations which are combined together with a sample to produce a bioluminescent assay composition. This assay composition, after an appropriate incubation period, has a BRET ratio that is indicative of an extracellular analyte in the sample, according to the principle disclosed herein. The BRET reagent system comprises (i) a solid composition which is dispersible in a liquid comprising the sample (e.g. the liquid sample) to produce the assay composition and (ii) a room temperature-stable substrate solution for dispensing into the assay composition. The solid composition comprises a biosensor system selected from (i) an extracellular bioluminescent sensor molecule comprising a donor lumiphore, an analyte-sensitive domain susceptible to interaction with the extracellular analyte, and an acceptor fluorophore; and (ii) a precursor system thereof comprising a pair of molecules adapted to react in solution to form the extracellular bioluminescent sensor molecule. The room temperature-stable substrate solution comprises at least one caged substrate compound, which is adapted to uncage in in the assay composition to produce an uncaged substrate compound which energises the donor lumiphore.

The solid composition may be contained in an assay vessel configured to receive the liquid comprising the sample and to permit transmission of light from the assay composition through the assay vessel walls for detection. The assay vessel may be according to any of the embodiments disclosed herein in the context of the system for detecting an extracellular analyte in a sample.

The solid composition may be a freeze-dried composition prepared by freeze-drying an aqueous precursor composition in the assay vessel, as previously disclosed herein.

The extracellular bioluminescent sensor molecule and any optional components in the solid composition may be according to any of the embodiments disclosed herein in the context of the method for detecting an extracellular analyte in a sample. In some embodiments, the solid composition further comprises an uncaging agent, such as an esterase, for uncaging the at least one caged substrate compound in the assay composition, as previously disclosed herein.

The room temperature-stable substrate solution may be contained in a substrate storage vessel configured to dispense an aliquot of the substrate solution into the assay composition. The substrate storage vessel may be according to any of the embodiments disclosed herein in the context of the system for detecting an extracellular analyte in a sample. In some embodiments, substrate storage vessel is configured to dispense the substrate solution drop-wise into the assay composition.

The caged substrate compound(s) and solvents in the room temperature-stable substrate solution may be according to any of the embodiments disclosed herein in the context of the method for detecting an extracellular analyte in a sample. In some embodiments, the room temperature-stable substrate solution comprises at least two different caged substrate compounds adapted to uncage in the assay composition to produce the uncaged substrate compound at different reaction rates.

In some embodiments, the BRET reagent system is a kit, comprising instructions for use of the BRET reagent system in a method for detecting an extracellular analyte in a sample according to any embodiment disclosed herein.

The present invention further relates to a solid biosensor composition for BRET detection of an extracellular analyte in a sample. The solid biosensor composition comprises (a) a biosensor system selected from (i) an extracellular bioluminescent sensor molecule comprising a donor lumiphore, an analyte-sensitive domain susceptible to interaction with the extracellular analyte, and an acceptor fluorophore; and (ii) a precursor system thereof, the precursor system comprising a pair of molecules adapted to react in solution to form the extracellular bioluminescent sensor molecule; and (b) an uncaging agent. In use, the solid biosensor composition is dispersible in a liquid comprising the sample (e.g. in a liquid sample) to produce an assay composition. The uncaging agent is adapted to uncage at least one caged substrate compound in the assay composition, thereby producing an uncaged substrate compound which energises the donor lumiphore according to the principles disclosed herein. The solid biosensor composition may optionally comprise other components such as (i) buffers, (ii) one or more salts, (iii) one or more metal ions, (iv) one or more carrier proteins, and (v) one or more cryoprotectants as described herein.

It will be appreciated that the solid biosensor composition is suitable for use in a method for detecting an extracellular analyte in a sample, as disclosed herein. The extracellular bioluminescent sensor molecule, the uncaging agent and the optional other components may generally be according to any embodiments disclosed herein in the context of this method and other aspects and embodiments of the invention.

The present invention further relates to a substrate dispensing system for BRET detection of an extracellular analyte in a sample. The substrate dispensing system comprises a room temperature-stable substrate solution comprising at least one caged substrate compound. The substrate solution is contained in a substrate storage vessel configured to dispense a pre-determined aliquot (e.g. one or more drops) of the substrate solution into an assay composition comprising the sample and a BRET bioluminescent sensor molecule.

It will be appreciated that the substrate dispensing system is suitable for use in a method for detecting an extracellular analyte in a sample, as disclosed herein. The at least one caged substrate compound and the solvents in the room temperature-stable substrate solution may generally be according to any embodiments disclosed herein in the context of this method. In some embodiments, the room temperature-stable substrate solution comprises at least two different caged substrate compounds adapted to uncage at different reaction rates.

The present invention further relates to a substrate composition for BRET detection of an extracellular analyte in a sample. The substrate composition comprises at least two different caged substrate compounds adapted to uncage in an assay composition comprising the sample and a BRET bioluminescent sensor molecule, thereby producing an uncaged substrate compound at different reaction rates. The substrate composition may generally be according to any embodiments disclosed herein in the context of this method and other aspects and embodiments of the invention.

It will be appreciated that the substrate composition is suitable for use in a method for detecting an extracellular analyte in a sample, as disclosed herein. The caged substrate compounds may generally be according to any embodiments disclosed herein in the context of this method.

### EXAMPLES

The present invention is described with reference to the following examples. It is to be understood that the examples are illustrative of and not limiting to the invention described herein.

### Materials and Methods.

All chemicals, reagents and enzymes were obtained from Sigma-Aldrich unless specified otherwise.

Caged bisdeoxycoelenterazine compounds 7, 8 and 10 were purchased from a custom organic synthesis company (Advanced Molecular Technologies, Australia) according to a previously reported synthesis methodology (J Am Chem Soc 2007 129(39) 11900-11901).

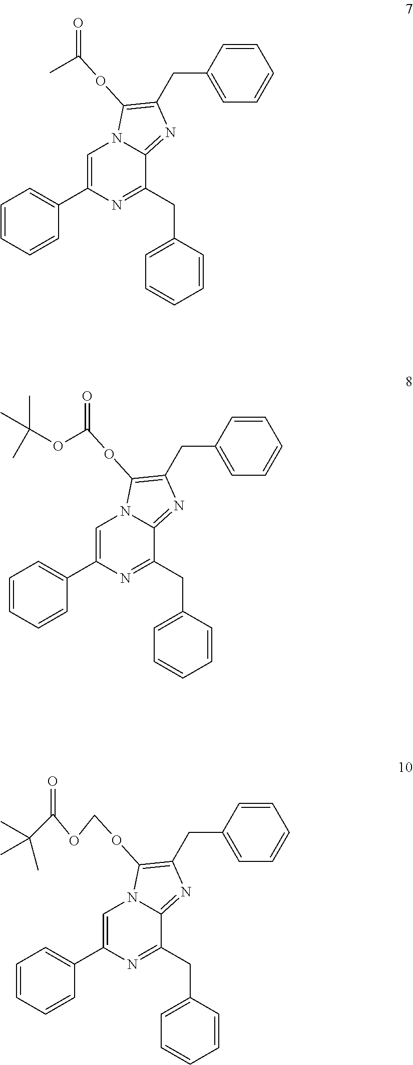 [SMILES: CC(=O)Oc1c(Cc2ccccc2)nc2c(Cc3ccccc3)nc(-c3ccccc3)cn12 ; CC(C)(C)OC(=O)Oc1c(Cc2ccccc2)nc2c(Cc3ccccc3)nc(-c3ccccc3)cn12 ; CC(C)(C)C(=O)OCOc1c(Cc2ccccc2)nc2c(Cc3ccccc3)nc(-c3ccccc3)cn12]

Bisdeoxycoelenterazine was Obtained from Nanolight Technology (USA).

An AprX bioluminescent sensor molecule (hereafter AprX sensor) was constructed as described in published US Patent application no. US2019/0345535 A1 (specifically Example 1 and Sequence ID no. 105 of that application; see SEQ ID NO: 1 of the present application) to generate the pRSET-Pflu expression plasmid. The AprX sensor was expressed and purified as follows.

One tube of chemically-competent E. coli BL21 (DE3) (New England Biolabs) was taken from −80° C. and placed on ice for 10 min. The overexpression plasmid pRSET-Pflu prepared as described above, was taken from the −80° C. freezer and placed on ice. 1-2 μl of the pRSET-Pflu plasmid was added to the thawed tube of BL21 (DE3) competent cells, mixed gently and incubated for 30 min on ice. The transformation mix was heat-shocked by placing the tube at 42° C. for exactly 10 sec. The transformation mix was placed on ice for 3 min and then 900 μl of sterile LB (Table 1) was added and the transformation mix was incubated at 37° C. for 1 h.

TABLE 1
Buffered LB medium

| Chemical | Concentration | |
|---|---|---|
| Tryptone | 10 | g/L |
| Yeast extract | 5 | g/L |
| NaCl | 10 | g/L |
| pH | 7.0 | (NaOH) |

100 μl and 20 μl aliquots of the reaction mix were plated out on agar, supplemented with 100 μg/ml ampicillin and 1% (v/v) glucose) and incubated at 37° C. for 24 h.

To produce AprX sensor protein, one colony of transformed E. coli BL21 (DE3) transformed with pRSET-Pflu was picked into 10 mL LB medium mixed with 1 ml 20% (w/v) glucose and supplemented with 100 μg/mL ampicillin and incubated at 37° C. for 18 h with orbital shaking (200 rpm). The entire 10 mL overnight culture was added to 500 ml buffered 2×YT medium (Table 2) in a 3 L sterile flask supplemented with ampicillin 100 μg/mL and incubated at 37° C. for 3 h with orbital shaking (200 rpm) until the culture reached an optical density at 600 nm of ~0.5.

TABLE 2
Buffered 2xYT medium

| Chemical | Concentration | |
|---|---|---|
| Tryptone | 16 | g/L |
| Yeast extract | 10 | g/L |
| NaCl | 5 | g/L |
| HEPES | 50 | mM |
| pH | 7.5 | (NaOH) |

250 μL of a 1 M stock solution of Isopropyl β-D-1-thiogalactopyranoside (IPTG, final concentration 500 μM) was added to the flask and the incubation temperature was adjusted to 25° C. for 18-24 h. The bacterial cells were pelleted by centrifugation (20 min, 4,000 g) and the supernatant was discarded. The cell pellet was resuspended in 50 mL phosphate buffered saline (PBS; Table 3) per 200 mL starting culture.

TABLE 3
Phosphate buffered saline (PBS)

| Chemical | Concentration | |
|---|---|---|
| NaCl | 137 | mM |
| KCl | 2.7 | mM |
| Na₂HPO₄ | 10 | mM |
| KH₂PO₄ | 1.8 | mM |

The cells were centrifuged (20 min, 4,000 g), the supernatant was discarded and the pellet was resuspended in a total of 100 mL buffer HisA (Table 4).

TABLE 4
Buffer HisA

| Chemical | Concentration | |
|---|---|---|
| NaCl | 500 | mM |
| Imidazole | 10 | mM |
| Tris | 50 | mM |

| | |
|---|---|
| pH | 8.0 |

The cells were lysed using lysozyme and sonication. The resuspended bacterial cells were transferred to a 100 ml Schott bottle. A 3 mm diameter spatula load of chicken egg lysozyme was added to the cell suspension and mixed thoroughly. 50 μL of 200 mM phenylmethylsulphonyl fluoride (PMSF) in dry ethanol was added to the suspension which was mixed thoroughly and placed on ice. A Misonix 3000 sonicator probe was placed into the suspension and the suspension was sonicated as follows: (3×30 seconds at 51 watts with the bottle being rotated ≈120° in between, followed by 5 minutes of standing on ice) performed three times. An additional 50 μl of 200 mM PMSF was added to the lysate and mixed thoroughly and the lysate was centrifuged at 15,000 g for 20 minutes at 4° C.

The AprX sensor protein was purified by metal ion chelate affinity chromatography. The supernatant following lysis and centrifugation was decanted into a clean container and held on ice. TALON immobilized metal affinity chromatography columns (TakaraBio) were prepared according to the manufacturer's instructions and loaded with the clarified, lysed cell supernatant. The tube was filled almost to the top with the supernatant, sealed and inverted several times. The top seal was removed and resin was allowed to settle under gravity for 10 minutes. Then the column bottom seal was removed and the lysate was drained off. The loading and settling process was repeated with the remaining unprocessed supernatant until it had all been applied. The column was washed with 20 mL of HisA buffer following the same procedure as for loading. The column was washed once with 3 mL of HisC buffer (Table 5) without disturbing the resin.

TABLE 5
Buffer HisC

| Chemical | Concentration | |
|---|---|---|
| NaCl | 500 | mM |
| Imidazole | 30 | mM |
| Tris | 50 | mM |

| | |
|---|---|
| pH | 8.0 |

The AprX sensor was eluted from the TALON column using 4 ml of HisB buffer (Table 6) and was stored at 4° C., in the dark.

TABLE 6
Buffer HisB

| Chemical | Concentration | |
|---|---|---|
| NaCl | 500 | mM |
| Imidazole | 300 | mM |
| Tris | 50 | mM |

| | |
|---|---|
| pH | 8.0 |

The purified AprX sensor protein was diluted to 100 nM in AprX storage buffer (Table 7), aliquoted into 1 mL portions, snap-frozen in liquid nitrogen and stored at −80° C.

TABLE 7
AprX storage buffer

| Chemical | Concentration | |
|---|---|---|
| NaCl | 100 | mM |
| Tris | 100 | mM |
| Cold water fish skin | 0.45% | (w/v) |
| gelatin (Sigma- | | |
| Aldrich, G-7765) | | |
| pH | 8.4 | (HCl) |

UHT reference milk (Devondale full cream UHT milk, best before date 10 Dec. 2021, purchased from IGA supermarket in O'Connor ACT or Devondale full cream UHT milk, best before date 17 Sep. 2022, purchased from Daily Market Groceries store, ANU Campus ACT) was frozen in aliquots and stored at −80° C. for up to 12 months before use.

Esterase from porcine liver was obtained from Sigma-Aldrich (cat no. E3019-20KU).

Alkaline protease (AprX) from Pseudomonas fluorescens was prepared as follows. An isolate of Pseudomonas fluorescens (Strain 65) [P. D. Button, H. Roginski, H. C. Deeth, H. M. Craven, Improved shelf-life estimation UHT milk by prediction of proteolysis, Journal of Food Quality, 34 (2011) 229-35.] was obtained from the Dairy Culture Collection at the CSIRO Food Innovation Centre, Werribee and was spread on OXOID agar plates and incubated at 28° C. for 2 days. 100 mL of sterile OXOID nutrient broth (NB) was inoculated with three colonies from the plate and incubated for 48 hr at 28° C. 10 mL of the culture was centrifuged at 6,000 g for 10 minutes. The supernatant was discarded and the pellet was re-suspended in 1 mL of sterile isotonic saline solution (0.85% w/v) and added to 99 mL skimmed UHT milk (COLES Australian skimmed milk, 99% fat free). The culture was incubated for 8 days at 4° C. with circular agitation at 150 rpm. Following this, the cultures were centrifuged at 27,000 g for 20 minutes at 4° C. The supernatant was collected, filter sterilised through a 0.22 μm filter and used as a source of AprX protease. 1 mL aliquots of the protease preparation were snap-frozen in liquid nitrogen and stored at −80° C.

Alkaline protease (AprX) activity was standardised based on measurements made using the azocasein method, as follows. Azocasein (A2765) was purchased from Sigma-Aldrich (Lot #SLBR0712V, specific E1% absorbance at 440 nM of 35). A 3% (w/v) working stock was prepared by gradually dissolving 90 mg azocasein in 750 μl 0.1 M NaOH followed by addition of 750 μL H₂O and eventually 1500 μL 50 mM Na₂HPO₃ (pH 7.5). AprX serial dilutions were prepared in 400 μL 50 mM Na₂HPO₃ (pH 7.5) in 1.5 ml Eppendorf tubes and mixed with 100 μl 3% (w/v) Azocasein solution. Tubes were incubated at 37° C. for 15 min to 24 hrs. Reactions were stopped by addition of 500 μL 20% (v/v) trichloroacetic acid, followed by centrifugation at 14,000 rpm for 3 min. to pellet undigested azocasein. 500 μL of the supernatant was carefully removed and neutralised with 750 μl 1 M NaOH in a micro cuvette. The absorbance at 440 nm was measured using a SpectraMax Plus spectrophotometer (Molecular Devices, United States). The average rate of change in Abs440 nm over each incubation time was converted to enzyme activity, using the molecular mass and extinction coefficient of the azocasein batch and the time of the incubation. It was expressed in terms of micromoles of azocasein reacting per minute per mL of enzyme solution (Units per mL).

Luminance and BRET² ratio measurements in a plate-reader were performed according to the following basic procedure, with amendments as noted in the Examples.

50 μL of UHT milk standard were mixed with 29 μL of AprX Buffer B3 (Table 8), 10 μL of standardised AprX protease of known activity and 10 μL of 100 nM AprX biosensor protein in a well of a 96-well plate.

TABLE 8
AprX buffer B3

| Chemical | Concentration | |
|---|---|---|
| NaCl | 100 | mM |
| Tris | 100 | mM |
| MgCl2 | 10 | mM |
| CaCl2 | 10 | mM |
| pH | 8.4 | (HCl) |

The plate was incubated with a nominal setting of 50° C. in a pre-heated Inkubator 1000 (Heidolph Instruments, Germany) for 10 min. The plate was transferred to a CLARIOstar (BMG Labtech) plate-reader set to 30° C. with the following additional settings:

| | |
|---|---|
| Measurement type | Luminescence |
| Microplate name | Nunc 96 |
| No. of intervals | 10 |
| Interval time [s] | 1.78 |
| Measurement interval time [s] | 0.5 |
| Focal height [mm] | 12.0 |
| Filter (Donor blue) | 410-80 (Gain:3700) |
| Filter (Acceptor green) | 515-30 (Gain:4000) |

Immediately, 1 μl of substrate e.g. CLZ400a (500 UM in dry ethanol) or caged substrate (6 mM in dry DMSO) was added. In the case of caged substrates, 1 μL of an aqueous solution of porcine esterase was also added. The contents of the well were mixed by pipetting and the reading protocol was started.

Human plasmin activity was standardised based on measurements made using Chromozyme PL using the protocol described below. Chromozyme PL is a chromogenic substrate containing a proteolytic recognition site for plasmin. The colorimetric change due to the plasmin-derived proteolytic release of dye from the chromozym PL substrate can be detected by measuring the absorbance at 410 nm. Human plasmin was purchased from Merck-Sigma-Aldrich (P1867), reconstituted in 50 mM Na₂HPO₄ (pH7.0), filter sterilised and aliquoted into sterile microcentrifuge tubes. All aliquots were snap-frozen in liquid nitrogen and stored at −80° C. Chromozyme PL was purchased from Merck-Sigma-Aldrich (10378461001). Assays were prepared in a total volume of 100 μL in a transparent 96-well plate, containing serial dilutions of plasmin (in 50 mM Na₂HPO₃, pH7.0). Chromozym PL was added to a final concentration of 0.315 mM and the plate was incubated in a CLARIOstar plate reader set to 37° C. Absorbance at 410 nm was measured every 2 min over a 30 min period. A control reaction without spiked plasmin was added to every group of tests. The absorbance difference (AA/min) between 0 and 4 min was determined and used to calculate plasmin activity in units, defined as

1 U=ΔA/min×Total Assay Volume/(Sample volume×Path length×extinction coefficient),

where the path length was 0.28 cm and the extinction coefficient was 9.7.

### Example 1. Storage Stability of Caged Deoxycoelentarazine Compounds in Solution

To be usable in a reliable on-the-spot BRET² test protocol, it was determined that a solution of the substrate compound when stored at 22° C. should support an emission amplitude of no less than 10% of its original value whilst maintaining a BRET² ratio within ±5% of its original value. Here, the emission amplitude is based on measurement of the Green Fluorescent Protein 2 (GFP²) emission and BRET ratios are calculated as Green Fluorescent Protein 2 (green emission)÷Renilla Luciferase 8 emission (RLuc8, blue emission). The substrate compound should be storable at room temperature for several weeks, and preferably storable for much longer periods such as up to a year.

The stability of a range of substrates for Renilla luciferase was tested as follows. Bisdeoxycoelenterazine (500 M) (95%) was prepared in dry ethanol and Bisdeoxycoelenterazine-HCl (CLZ400a-HCl) (500 μM) was prepared in dry DMSO. Separate solutions of compounds 7, 8 and 9 (each at 6 mM) were prepared in dry dimethylsulfoxide (DMSO). To conduct aging tests, including under elevated temperature conditions to accelerate aging, the substrate solutions were stored dry in o-ring sealed 2 mL tubes (QSP™ Natural Screw Cap Micro Tubes 520-GRD, ThermoFisher Scientific) placed in zip-lock resealable plastic bags containing a 5 g Minipax® absorbent pack (Sigma-Aldrich, Z163635) in the dark at a range of temperatures and tested at various times in a BRET² assay using the AprX sensor.

To assess substrate shelf-stability, the basic AprX sensor assay protocol (described above) was modified as follows to measure the luminance and BRET² ratios supported by the caged or uncaged substrates after they had been stored at various temperatures for various times. For CLZ400a itself, the standardised AprX protease was omitted from the reaction mix and replaced with an additional 10 μL of AprX Buffer B3. The assay mix was incubated for 10 mins at 50° prior to addition of CLZ400a or CLZ400a-HCl and was then measured immediately.

To assess caged substrates, the following modifications to this procedure were made. For compound 7, the AprX protease was replaced with 10 μL of AprX Buffer B3. Compound 7 was added in 1 μL of dry DMSO to a final concentration of 60 μM. The assay was then incubated for ten minutes at 50° C. For compounds 8 and 10, or mixtures thereof, the same procedure was used as for compound 7 except that the AprX protease addition was replaced with 9 μL of AprX Buffer B3. The caged substrate was added in 1 μL of dry DMSO to a final total concentration of 60 μM, followed immediately by 1 μl of an aqueous solution of porcine esterase (2 U/μL) to a final concentration of 20 U/mL. The assay was then incubated for ten minutes at 50° C. before being read on the Clariostar.

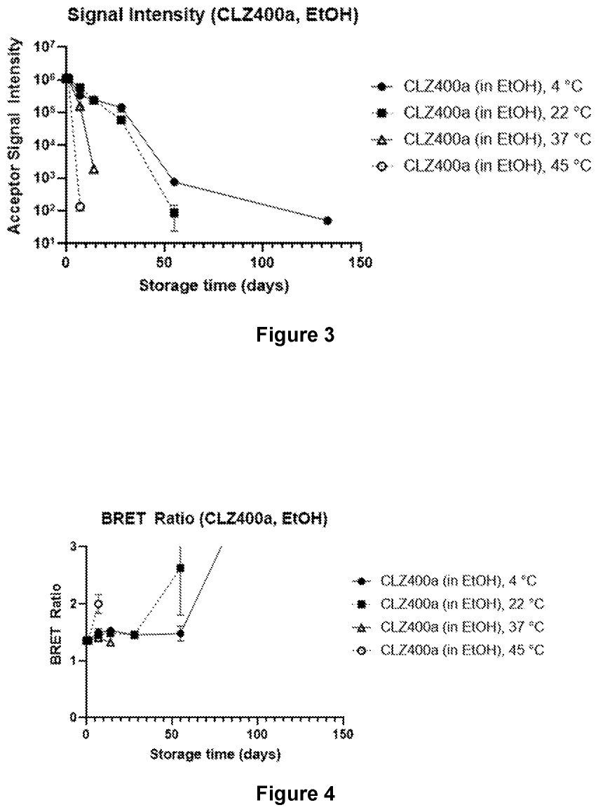

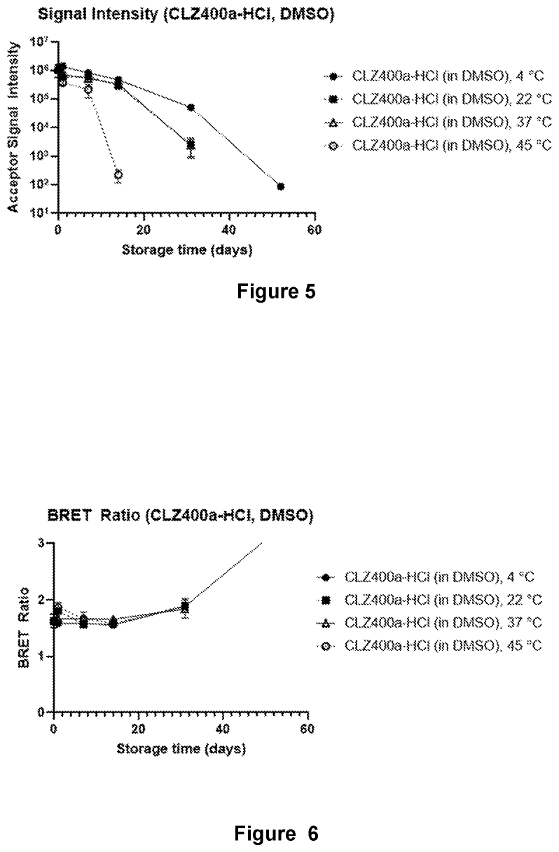

The results for uncaged bisdeoxycoelenterazine (CLZ400a or CLZ400a-HCl) are shown in FIGS. 3-6. Bisdeoxycoelenterazine produces the most intense light of all the substrates tested. However, at all temperatures, within 55 days, the bisdeoxycoelenterazine solution in ethanol (FIG. 3), and within 31 days the bisdeoxycoelenterazine-HCl solution in DMSO (FIG. 5) lost more than 90% of their capacity to energise the AprX biosensor. Uncaged bisdeoxycoelenterazine therefore did not meet the luminance stability requirement at any temperature because, a 90% loss of luminance results in an unstable BRET ratio and unacceptably high coefficient of variance in the ratio due to stochastic variation in photon counts at low luminance.

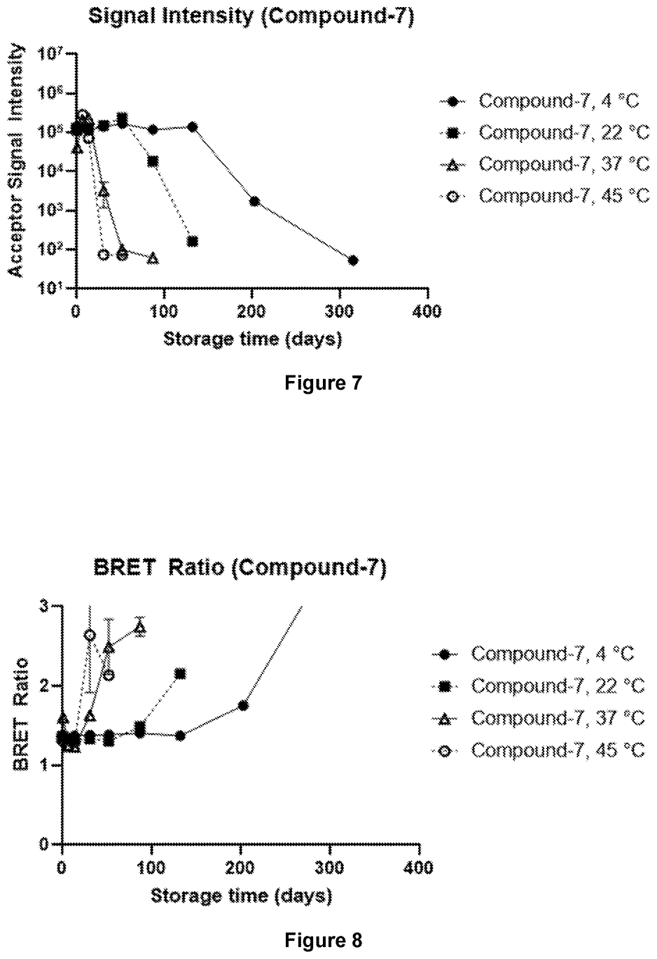

The results for caged bisdeoxycoelenterazine compound 7 are shown in FIGS. 7 and 8. Compound 7 was more stable in solution than uncaged bisdeoxycoelenterazine, and still met the luminance and BRET ratio criteria when stored at 4° C. for up to 132 days and at 22° C. for up to 80 days. However, the loss of both luminance and BRET ratio experienced in the elevated temperature samples predicts that solutions of compound 7 would no longer meet acceptability criteria after storage at greater than 4° C. for a few months. Nevertheless, Compound 7 has potential utility in supporting rapid uncaging if stored refrigerated for up to 132 days.

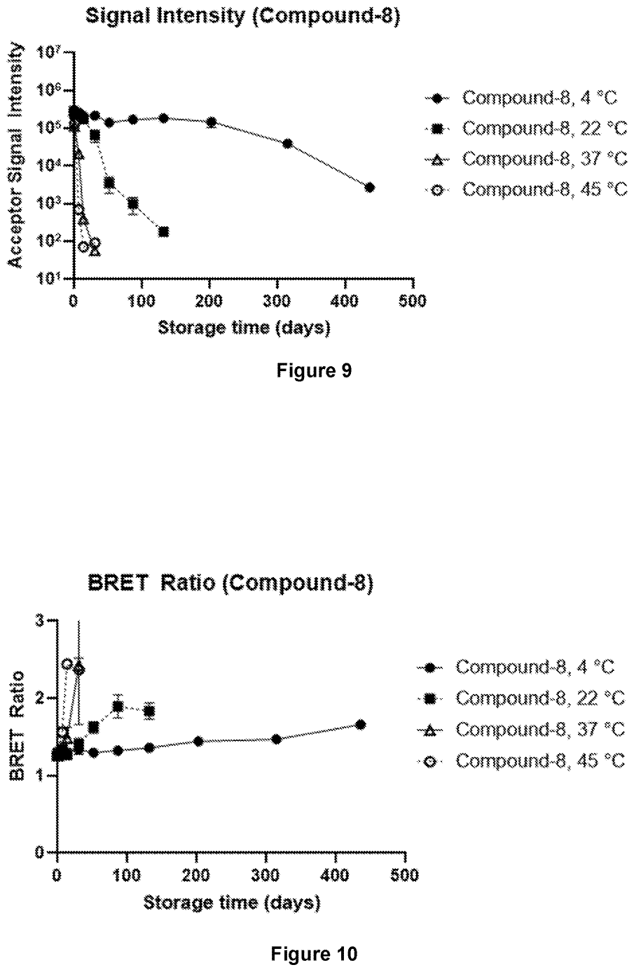

The results for caged bisdeoxycoelenterazine compound 8 are shown in FIGS. 9 and 10. Compound-8 was stable at 4° C. for up to 203 days after which its activity declined to ~13% of its initial capacity after 315 days. At all temperatures higher than 4° C., compound 8 solutions were stable for about two weeks, but they failed to meet both luminance and BRET ratio requirements after 40 days of storage, indicating that they could be used for timed release of uncaged substrates for up to one month.

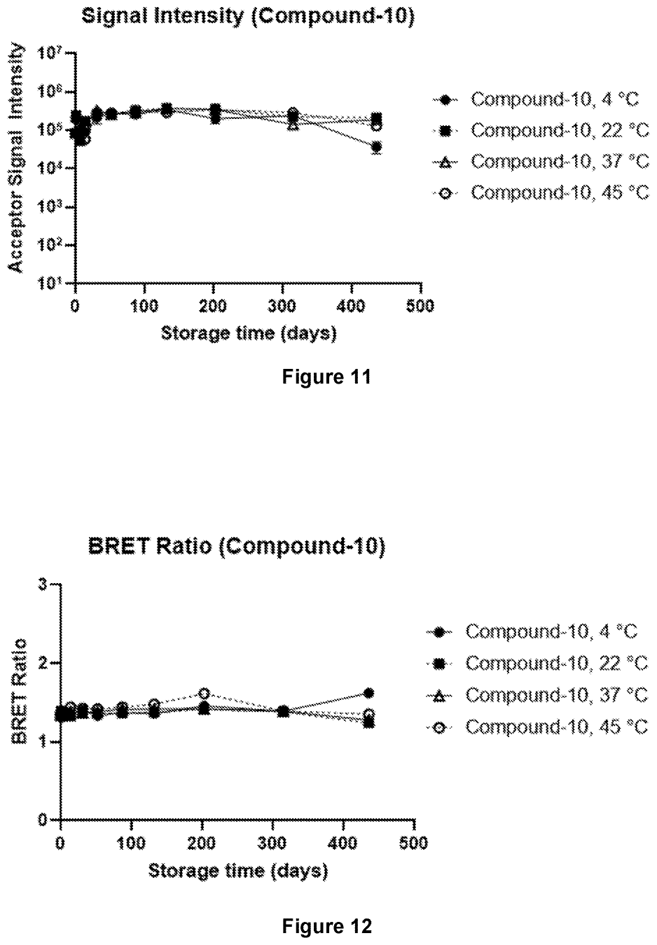

The results for caged bisdeoxycoelenterazine compound 10 are shown in FIGS. 11 and 12. Compound 10 maintained excellent stability in solution, meeting both luminance and BRET ratio criteria, for up to 300 days at 4° C. and at least 436 days at 22, 37 or 45° C.

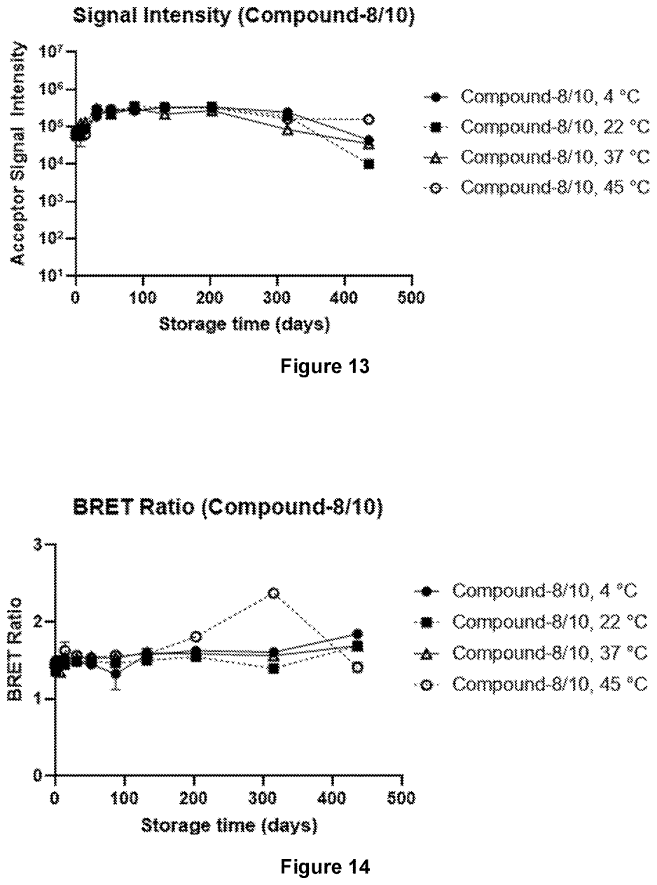

The stability of a DMSO solution containing a 1:2 mixture of caged compounds 8 and 10 (6 mM and 12 mM respectively) was also investigated (FIGS. 13 and 14). It was found that the 1:2 mixture provided a stable BRET ratio and good delayed bioluminescence signal onset for a 10 min assay in initial experiments (data not shown). However, different mixtures of caged substrates may be used to achieve optimally timed light generation for assays of varying durations. After apparent initial variation over 14 days, the luminance and BRET ratio of reactions energised by compounds 8 and 10 was stable at all temperatures (4° C., 22° C., 37° C. and 45° C.) after storing the solution for up to 315 days.

The results indicate that, caged coelenterazine compounds (but not uncaged coelenterazine) can be formulated to meet the requirements for a ready-to-use, medium to long shelf-life substrate solution for use in an on-the-spot BRET² assay for detecting analytes in liquid samples.

### Example 2. Timed Activation of BRET² Biosensors

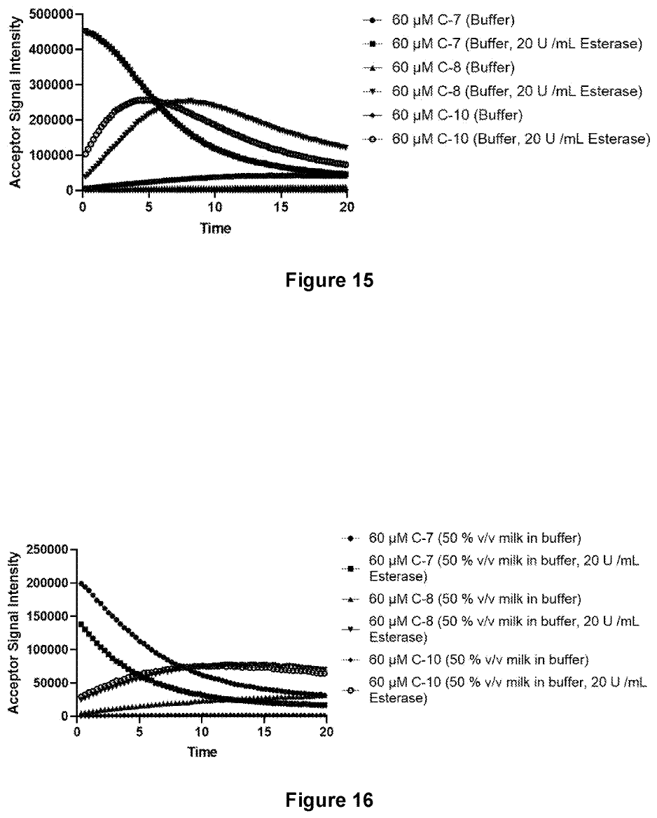

The susceptibility of compounds 7, 8 and 10 to decaging reactions was then investigated via BRET² assays (FIGS. 15 and 16). Compound 7 can be consumed by the BRET² sensor in buffer in the absence of esterase with a low and sustained bioluminescence signal. In UHT milk, compound 7 is activated in the absence of added esterase, exhibiting a delayed flash-type like bioluminescence. Compound 8 is also activated in milk but to a lesser degree, while compound 10 is not activated in milk. Both compounds 8 and 10 cannot be consumed by the BRET² sensor in absence of esterase in buffer. In milk and in buffer, both compounds 8 and 10 exhibit low but sustained bioluminescence signals in the presence of added porcine liver esterase with a longer activation delay compared to compound 7.

Preliminary experiments indicated that mixing one to three caged substrate components in combination with esterase enables different timed activation windows in which the BRET² peak intensity is reached at different times post mixing of all components in presence of the AprX sensor and dependent on the incubation temperature. 30 different assay compositions were tested (Table 9) for bioluminescence over time (0 to 20 min) on the CLARIOstar at 30° C. The assay compositions were as follows:
50 μl UHT milk (r00014)
0-4 μl porcine esterase
10 μl AprX sensor (100 nM stock)
33.5-37.5 μl Buffer b0002
2.5 μl substrate mixture (solution in DMSO).

The substrate mixture was added last, followed by immediately running a 63 cycle BRET² signal channels over 20 min. The amount of caged substrate compound(s) and exogeneous decaging agent in each assay composition is shown in Table 9.

TABLE 9

| | | | | Porcine |
|---|---|---|---|---|
| Assay | Compound 7 | Compound 8 | Compound 10 | esterase |
| code | [μM] | [μM] | [μM] | (Units/100 μl) |
| 1 | 60 | 60 | 60 | 4 |
| 2 | 30 | 60 | 60 | 4 |
| 3 | 10 | 60 | 60 | 4 |
| 4 | 5 | 60 | 60 | 4 |
| 5 | 1 | 60 | 60 | 4 |
| 6 | 60 | 60 | 60 | 2 |
| 7 | 30 | 60 | 60 | 2 |
| 8 | 10 | 60 | 60 | 2 |
| 9 | 5 | 60 | 60 | 2 |
| 10 | 1 | 60 | 60 | 2 |
| 11 | 60 | 60 | 60 | 1 |
| 12 | 30 | 60 | 60 | 1 |
| 13 | 10 | 60 | 60 | 1 |
| 14 | 5 | 60 | 60 | 1 |
| 15 | 1 | 60 | 60 | 1 |
| 16 | 60 | 0 | 0 | 4 |
| 17 | 30 | 0 | 0 | 4 |
| 18 | 10 | 0 | 0 | 4 |
| 19 | 5 | 0 | 0 | 4 |
| 20 | 1 | 0 | 0 | 4 |
| 21 | 0 | 60 | 60 | 1 |
| 22 | 0 | 60 | 60 | 2 |
| 23 | 0 | 60 | 60 | 4 |
| 24 | 0 | 60 | 60 | 8 |
| 25 | 0 | 60 | 60 | 0 |
| 26 | 0 | 120 | 120 | 1 |
| 27 | 0 | 120 | 120 | 2 |
| 28 | 0 | 120 | 120 | 4 |
| 29 | 0 | 120 | 120 | 8 |
| 30 | 0 | 120 | 120 | 0 |

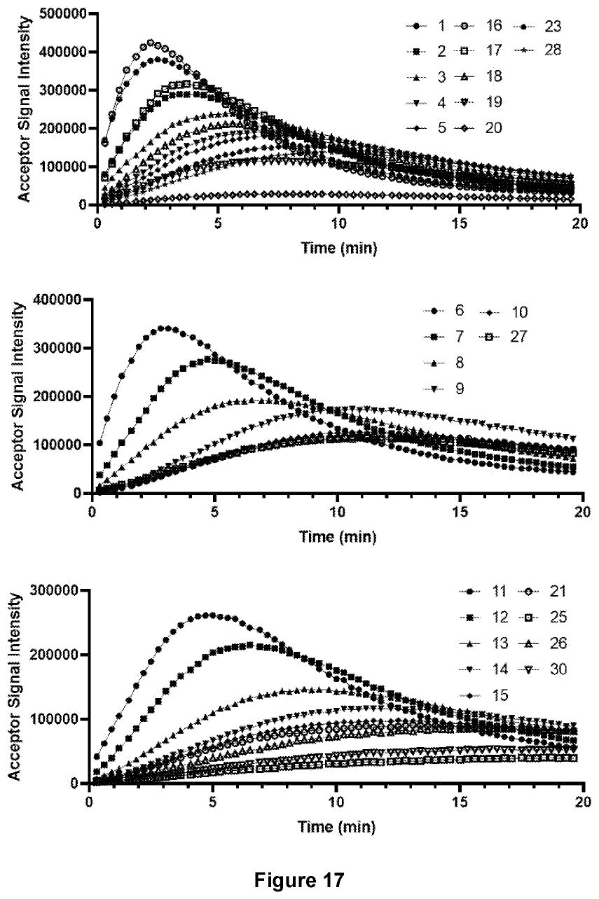

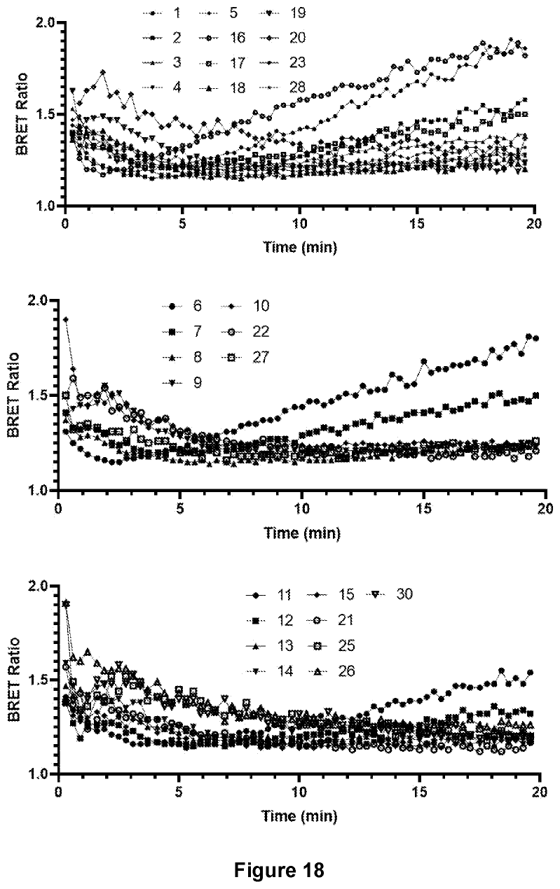

The signal intensities, based on measurement of the Green Fluorescent Protein 2 (GFP²) emission, and BRET ratios, calculated as GFP² (green emission)÷Renilla Luciferase 2 emission (RLuc2, blue emission), are shown in FIGS. 17 and 18 respectively. Three formulations (assay codes 20, 25 and 30) did not exhibit a signal peak intensity of >50,000 and they were discarded for following analyses.

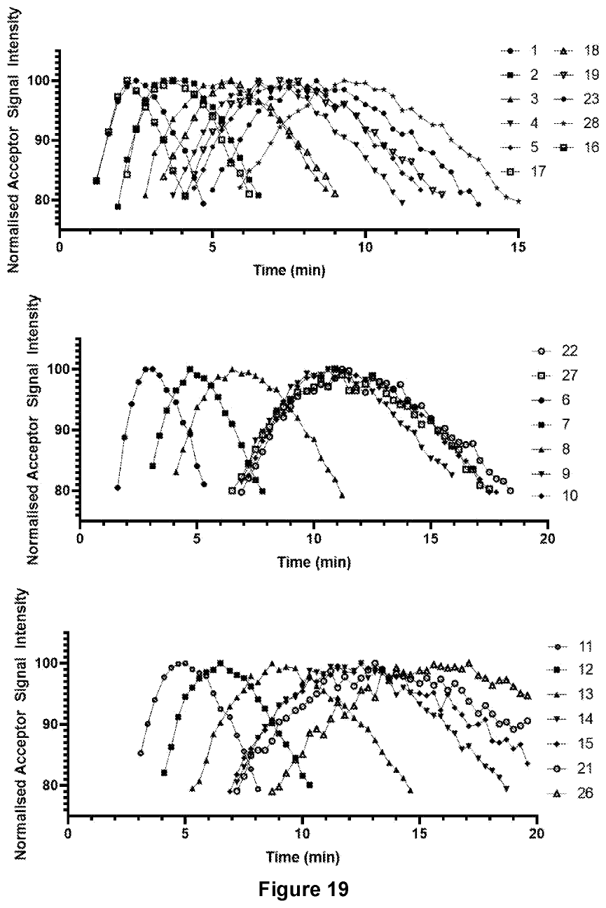

The BRET ratios were seen to vary dependent on the formulation and with time. However, the BRET ratios for each formulation were generally stable (i.e. a low coefficient of variation) around their 80-100% signal peak. To emphasise the assay time within which a formulation exhibits 80-100% of its signal peak, only those normalised signals are shown in FIG. 19. The normalised signal intensities illustrate clearly how different formulations lead to different substrate activation windows ranging from 1 min to 20 min.

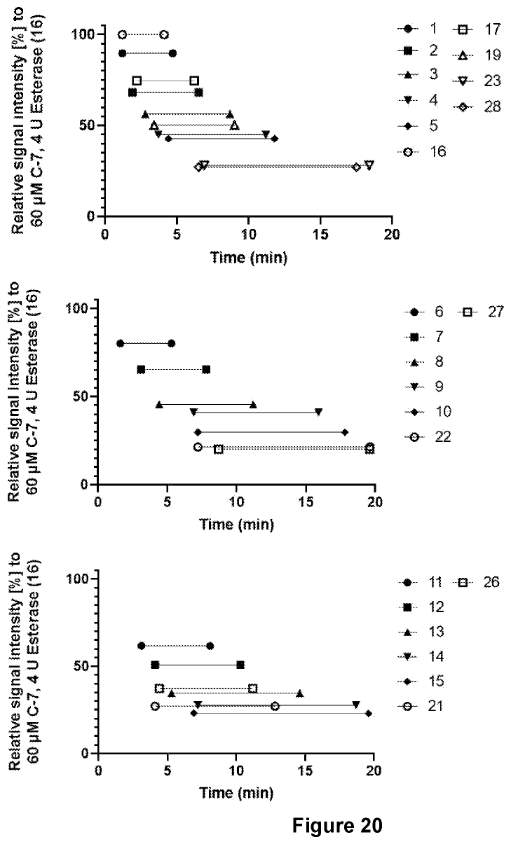

The information of FIGS. 17 and 18 respectively was then combined in FIG. 20 to show when the peak signal window (80-100% relative signal intensity) occurs for each formulation and at what peak intensity strength (normalised to the formulation with the highest signal peak intensity: 60 μM C-7, 4 U Esterase (Assay code 16). The peak signal intensity time window follows a trend where lower signal intensities are correlated to a later and longer sustained bioluminescence generation.

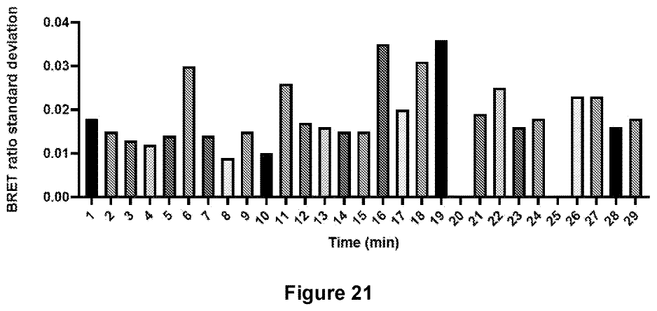

The BRET ratio stability was analysed by calculating the standard deviation of BRET ratios for each formulation within their 80-100% signal peak window, and the results are shown in FIG. 21. BRET ratio standard deviations vary from ~0.01 to 0.035. Most of the standard deviations were below 0.02, which is considered satisfactory for BRET² assay sensitivity.

Thus, for any given BRET² analysis having a known or preferred analysis time (e.g. for optimum interaction between the biosensor and analyte), the results of this experiment can be used to select a suitable caged substrate formulation, i.e. a formulation where (1) the analysis time falls within the 80-100% signal peak window and (2) preferably, the signal intensity is also as high as possible.

### Example 3. Storage-Stable Freeze-Dried Biosensor Formulations

The AprX biosensor was found to be stable in solution at 4° C. in the presence of the antimicrobial agent thymol. However, when a solution of AprX biosensor was stored at room temperature (22° C.), even in the presence of thymol, there was a steady decline in signal intensity and BRET² ratio over 80 days. Storing the biosensor in solution at ambient temperature for use in an on-the-spot assay methodology is therefore not considered viable. A study was thus conducted to determine if the AprX biosensor, together with other components including buffers, cryoprotectants and uncaging esterases, could be freeze-dried for long-term storage.

The required number of QSP™ Natural Screw Cap Micro Tubes 520-GRD (ThermoFisher Scientific) were charged with 200 μL of aqueous solution containing the following components (referred to herein as FD-B4):
250 mM Tris (pH 8.4)
250 mM NaCl
25 mM MgCl₂ 6×H₂O
25 mM CaCl₂ 2×H₂O
0.45% (v/v) fish skin gelatine (*The stock solution contains 45% (w/v) (0.45 g/mL) fish skin gelatine)
12.5 mg/ml trehalose
12.5 mg/ml pullulan
5 U/ml U/ml porcine esterase
3,450 ng/ml AprX biosensor

The uncapped assay tubes were held in a −80° C. freezer for at least 2 hours (although this snap-freezing preliminary step can also be omitted due to the gelatine/trehalose/pullulan components). An Alpha 1-2 LDplus freeze-dryer and attached oil vacuum pump were turned on and warmed up and the freeze-dryer's back plate was pre-chilled to −80° C. The back plate and sample tubes were loaded into the freeze-dryer and it was switched on for 20 hours, after which time the freeze-dryer was switched to standby mode, the vacuum was slowly released and the sample tubes were removed, capped and stored in the dark in a heat-sealed plastic bag containing a 5 g Minipax® absorbent pack.

A number of other freeze-drying protocols, using other equipment, were shown to be successful. These included a commercial-style protocol where the mix was cooled to ≈0° C., but not pre-frozen, prior to freeze-drying.

The freeze-dried compositions were then subjected to accelerated aging tests by storing the sealed assay tubes in the dark at temperatures of 4° C., 22° C., 37° C. and 45° C. for 9 months. There was no change in appearance or evidence of a glass to rubber transition for samples stored at 4° C. Samples stored at higher temperatures than 4° C. turned yellow after 6 months (22° C.), 3 months (37° C.) and 2 months (45° C.).

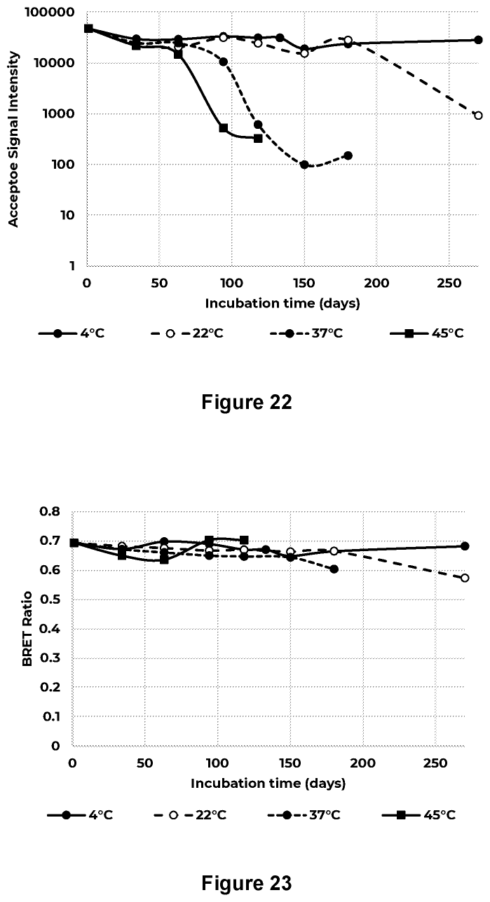

The performance of the freeze-dried biosensor compositions after ageing was then evaluated in BRET² assays on the optoelectronic device. The freeze-dried assay tubes were reconstituted with 1 ml raw milk, supplemented with 28 μL 4.3 mM compound 10 (in dry DMSO), mixed and inserted into the optoelectronic device. The device was set to 37° C. and signals were monitored over 15 min (FIGS. 22 and 23).

Signals were monitored on the alpha device from 0-118 days followed by analyses on the beta device (133-270 days). As the signal output changed in between both devices due to hardware changes, a correction factor of 0.19 for signal intensity values and a correction factor of 1.39 for BRET ratios from days 133-270 was applied to the data.

The freeze-dried biosensor showed some variation in overall intensity but no decline in BRET² ratio for over 9 months at 4° C., while signals remained stable for up to 6 months for samples stored at 22° C. The freeze-dried biosensor was stable at 37° C. for 2 months and at 45° C. for 1 month. BRET ratio stability was correlated to signal intensities, remaining stable until the drop in signal intensity.

### Example 4. Detection of AprX Activity in Milk Samples Using Shelf-Stable Biosensor Reagents

Examples 1-3 demonstrate the path towards the creation of shelf-stable assay reagents that can be used in analyses (e.g. of milk) with an incubation time of 2-20 min. Next, we tested whether these assay reagents can be applied to a variety of milk samples on an optoelectronic device as described herein with reference to FIGS. 1 and 2, which is a bespoke point-of-need capable BRET analysis device. Therefore, a study was conducted producing shelf-stable AprX assay reagents and testing their performance in raw milk, ultra-heat-treated milk and pasteurised milk (referred to as fresh milk).

Freeze-dried AprX biosensor formulations were prepared in assay tubes using the same shelf-stable formulation (5 U/ml porcine esterase) and freeze-drying procedure determined in Example 3, except without the addition of pullulan (which has no effect on the assay other than stabilising the reagents for long-term storage).

The freeze-dried formulation was reconstituted with 1 mL raw milk (providing 1 U/ml porcine esterase in the reconstituted formulation), supplemented with serial dilutions of standardised AprX protease, and followed by the addition of 28 μL compound 10 solution (in dry DMSO, 4.3 mM) to provide an assay concentration of 120 μM compound 10. The tube was closed and inverted 20 times and inserted into the optoelectronic device.

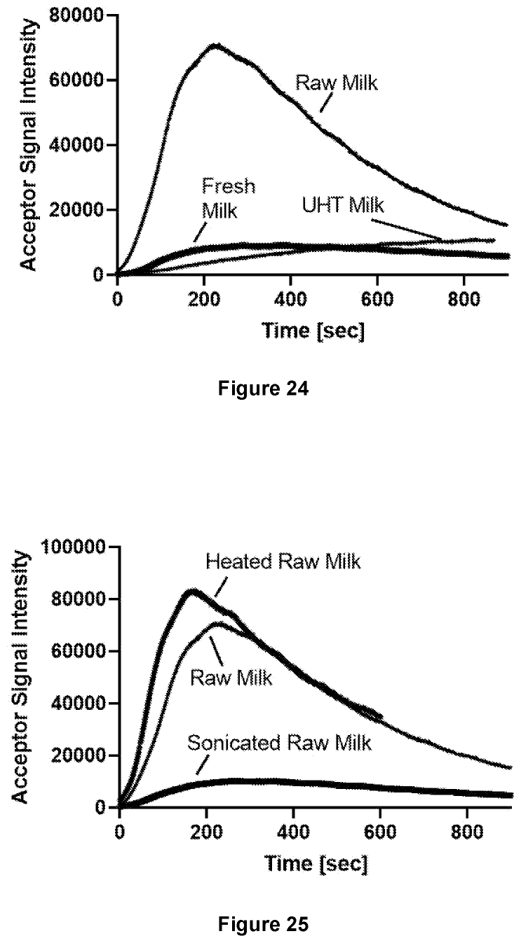

Initial assays (without addition of AprX protease) were performed using the optoelectronic device incubating at 37° C. and the BRET² bioluminescence signals were recorded over 900 sec (15 min). Assays performed in raw milk exhibited a strong signal peaking at around 250 sec followed by a slow signal decay, whereas using fresh milk or UHT milk led to a low and sustained signal generation (FIG. 24).

The increased bioluminescence generation in raw milk compared to fresh and UHT milk was also seen in raw milk samples heated at 95° C. for 5 min but it was not seen in raw milk samples sonicated at an amplitude of 40 for 1 min (QSonica, United States) (FIG. 25). Sonication homogenises raw milk samples. Without wishing to be bound by any theory, it is proposed that the caged substrate 10 is rapidly absorbed into the tiny fat globules present in homogenised milk, reducing the rate of decaging. Raw milk may also contain a thermostable but sonication/homogenisation prone factor affecting the bioluminescence generation of the biosensor with the caged substrate.

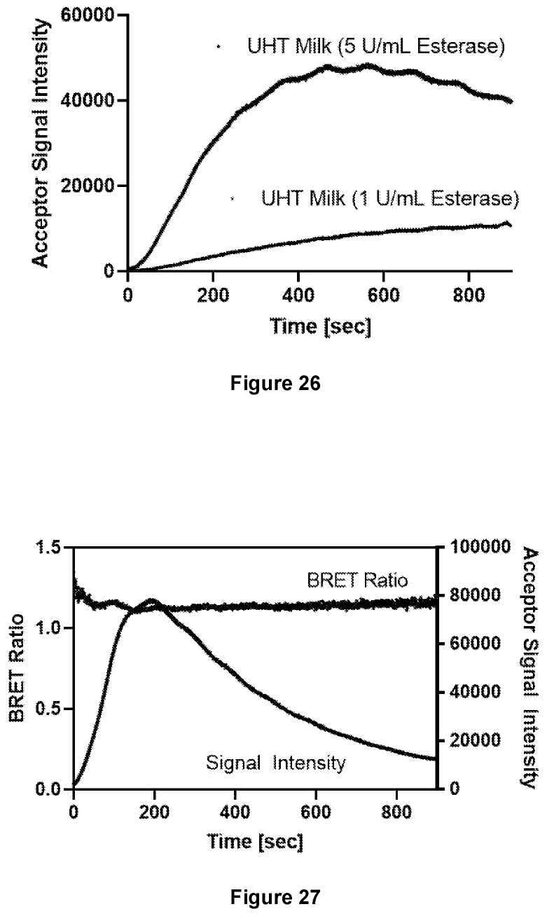

Adjusting the esterase concentration from 1 U/mL to 5 U/mL in the final assay resolves the low signal generation in UHT milk (FIG. 26). This indicates that different formulations are required for different milk sample types.

Unlike earlier observations using a plate reader, the optoelectronic device demonstrates more stable BRET ratios after reaching 100% peak luminescence window (FIG. 27). BRET ratios stabilise after 200 sec and remain stable for at least 15 min. On this device, end-point measurements may be reliably measured outside of the 80-100% peak luminescence window.

### Example 5. Calibration Curve for AprX Activity in Raw Milk Samples Using Shelf-Stable Biosensor Reagents

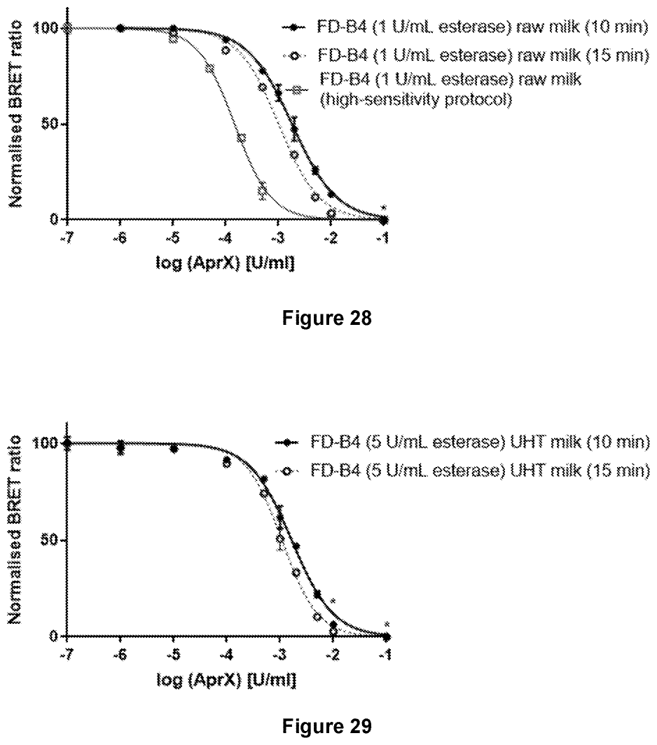

Using the BRET² protocol developed in Example 4, raw milk samples spiked with known amounts of standardised AprX protease were tested using the shelf-stable reagents containing 1 U/mL porcine esterase. Additionally, reagents containing 5 U/mL porcine esterase were tested with AprX-spiked UHT milk samples. Bioluminescence values from 590-600 sec were used to determine BRET ratio values for a 10 min assay and bioluminescence signal values recorded from 890-900 sec were used to determine BRET ratio values for a 15 min assay. The observed BRET ratios decreased with increasing amounts of AprX activity in the raw milk samples. Calibration plots for the 10 and 15 minute assays were fitted with non-linear calibration curves (FIGS. 28 and 29). The AprX activity detection was found to be dependent on the incubation time, where a 15 min assay was more sensitive compared to a 10 min assay. For raw milk, a 10 min assay achieved a limit of detection (LOD) of 83 μU/mL and a half maximal effective dose (EC₅₀) of 1.82 mU/mL, while a 15 min assay achieved an LOD of 67 μU/mL and an EC₅₀ of 1016 μU/mL. For UHT milk, a 10 min assay achieved a LOD of 232 μU/mL and an EC₅₀ of 1.65 mU/mL, while a 15 min assay achieved an LOD of 184 μU/mL and an EC₅₀ of 1084 μU/mL.

The detection sensitivity could be further improved by incubating reconstituted freeze-dried biosensor with spiked raw milk samples on a heating block (Delvo MiniS incubator) set to 34° C. for 1 hour before the addition of compound 10 (FIG. 26) (referred to as ‘high-sensitivity protocol’). Samples were then analysed for 15 min on the optoelectronic device and bioluminescence signal values recorded from 890-900 sec were used to determine BRET ratio values for the high-sensitivity protocol. The LOD for the high-sensitivity protocol was 17 μU/mL and the EC₅₀ was 148 μU/mL.

### Example 6. Use of a Preparation Station to Prepare Milk Analysis Samples

Next, we developed an easy-to-use workflow to prepare samples for the optoelectronic device used in the methods of Examples 3-5 that includes a single pipetting step to add a 1 mL liquid sample such as milk to a 2 mL screw cap tube containing freeze-dried biosensor system followed by applying one drop of a solution containing the caged substrate from a dropper bottle to start the bioluminescence reaction.

To enable operators with minimal training and less developed fine-motor skills to generate highly reproducible results, a preparation station was created. This station is designed to eliminate the final pipetting step and enhance user-friendliness, thereby simplifying the process of analysing samples on the optoelectronic device.

The preparation station, as shown in FIG. 30A, comprises the following components:
A dispensette dispenser designed to extract milk samples from a cup.
A linear rail system that facilitates the placement of an opened reagent tube and allows for the movement of the tube among three different positions.
A cylindrical cavity that accommodates a removable dropper bottle holder containing a dropper bottle filled with substrate solution (FIG. 30B). The bottle holder can be positioned over the tube located within the rail system, enabling the dispensing of a drop of substrate solution into the reagent tube. Once the dropper bottle holder is inserted, the cavity becomes airtight and includes provisions for fitting a desiccant pad.
   We next compared the performance of specific components of the preparation station and the workflow described in examples 3-5.

Firstly, we assessed the performance of the dropper bottle holder by quantifying the dispensed volume (where 1 mg equates to 1 μL) from a dropper bottle containing 2 mL of DMSO. This assessment was conducted through 20 repetitions, both manually and when operated within the dropper bottle holder (FIG. 30C). The dropper bottle holder consistently dispensed 28.9±0.7 μL of DMSO, while manual operation of the dropper bottle resulted in a slightly lower dispensed volume of 27.8±0.9 μL.

Similarly, we compared the performance of the dispensette with that of a fixed-volume 1 mL pipette for the task of drawing milk from a sample cup (FIG. 30D). The fixed-volume pipette dispensed a slightly smaller quantity of milk, with measurements at 975.4±6.8 μL, in contrast to the dispensette, which consistently dispensed 1027.7±2.6 μL.

It is worth noting that both elements of the sample preparation station, namely the dispensette and the dropper bottle holder, exhibited greater reproducibility in their dispensing performance compared to their manual operation counterparts (FIG. 30E).

We then compared the assay performance (Signal intensity and BRET ratio) analysing UHT full cream milk samples with FD-B4 (1 U/assay esterase, 5 U/ml esterase prior to freeze drying) as described in example 5. Samples were either prepared using the preparation station or via the manual workflow (1 mL fixed volume pipette and manual operation of the dropper bottle) (FIG. 30F and FIG. 30G). Signal intensities were slightly lower compared to the manual workflow when the preparation station was used (FIG. 30F). This could be due to the higher amount of dispensed milk influencing the amount of attenuated bioluminescence signal. Importantly, no difference in BRET ratios was observed between both sample preparation methods (FIG. 30G).

The dropper bottle holder enables the user to store the dropper bottle in a light-protected and dry location. We tested whether the substrate (compound-10, 4.3 mM in dry DMSO) remains stable at room temperature within the holder cavity. This was tested using FD-B4 (5 U/assay esterase; 25 U/mL esterase prior to freeze drying) reagents with raw milk samples in combination with a drop from the dropper bottle stored within the preparation station (as described in example 5). Over 16 weeks we did not observe any notable drop in signal intensity (FIG. 30H) or change in BRET ratio (FIG. 30I).

The preparation station offers a simplified assay preparation workflow for operators with minimal training without compromising assay performance. The storage stability of the substrate solution within the preparation station further improves the ease-of-use of the overall system.

The preparation station may also be conveniently used to dilute samples, prior to analysis with a biosensor, for example when using a high sensitivity BRET-based lactose sensor to measure the lactose concentration in milk that has not been treated with lactase enzyme or where the hydrolysis is at an early stage.

### Example 7. Formulation of and Running of a Lactose Detection Test on the Optoelectronic Device

Next, we tested whether the optoelectronic device described herein was compatible with a shelf stable lactose assay. This example describes the development of this assay and the development of shelf-stable two component reagent system.

The BRET² lactose sensor molecule has previously been described (see Australian patent 2018315053; Caron, K. and S. C. Trowell (2018). “Highly Sensitive and Selective Biosensor for a Disaccharide Based on an AraC-Like Transcriptional Regulator Transduced with Bioluminescence Resonance Energy Transfer.” Anal Chem 90 (21): 12986-12993; see also SEQ ID NO: 2 herein). Application of the BRET² lactose sensor molecule in a non-shelf-stable assay has also been described (Briggs, L., et al. (2023). “On-the-spot trace lactose test for flavored and unflavored lactose-free dairy beverages.” Talanta Open 8). The lactose biosensor comprises the native or a modified lactose-binding domain, from the BgaR transcription factor, sandwiched in between RLuc2 and GFP². Binding of lactose results in a conformational change of the lactose-binding domain that in turn alters the distance and/or orientation between RLuc2 and GFP² components, thus altering the energy transfer efficiency between RLuc2 and GFP². The sensor can be used to quantify the lactose content of dairy products.

It had been observed in unpublished experiments that the dynamic range of the change in sensor BRET ratio on lactose binding (determined by comparing the ratio in lactose-free milk with the ratio in lactose-free milk with 5% (w/v) lactose added) decreased over time at 4° C. It was hypothesised that the sensor dimerised over time due to a dimerisation domain located within the lactose-binding domain. Based on the dimerisation surface deduced from the crystal structure (Newman, J., et al. (2019). “Structures of the transcriptional regulator BgaR, a lactose sensor.” Acta Crystallogr D Struct Biol 75 (Pt 7): 639-646) several variants harbouring mutations within the dimerisation domain were constructed and three were selected for further study (LacB6 [GFP²-BgaR(D140A/I141A)-RLuc8], SEQ ID NO: 3, LacB13 [GFP²-BgaR(N12S/D140S/I141A)-RLuc8], SEQ ID NO: 4 and LacB16 [GFP²-BgaR(D140S/1141A/L142A/L144A)-RLuc8], SEQ ID NO: 5). The wild-type and variant lactose sensors were expressed and purified as described previously.

The changes over time in the dynamic range of the BRET ratio for the ‘wild-type’ lactose biosensor and the three variants was measured using a modified plate-reader assay. Briefly, 89 μL of AprX Buffer B3 (Table 8) was mixed with 10 μL of 100 nM lactose biosensor protein in a 96-well plate. The plate was incubated with a setting of 30° C. in a pre-heated Inkubator 1000 (Heidolph Instruments, Germany) for 5 min. The plate was transferred to a CLARIOstar (BMG Labtech) plate-reader set to 30° C. with the settings described in the methods section). 1 μL of CLZ400a substrate (500 μM in dry ethanol), was added, mixed by pipetting and the BRET ratio was measured.

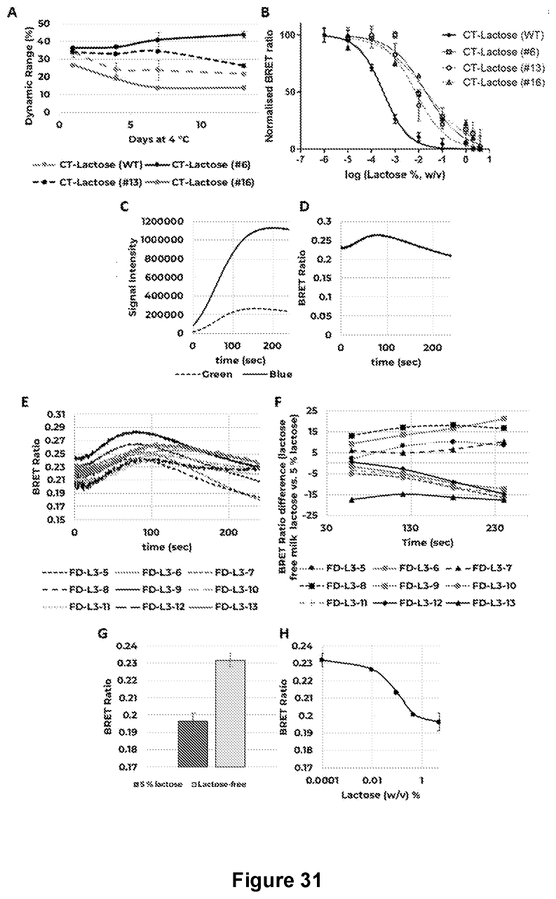

The dynamic range of the wildtype, LacB6 and LacB16 biosensor variants changed over time. In contrast, the LacB13 biosensor had a stable dynamic range for 7 days, followed by a drop in dynamic range (FIG. 31A). Without wishing to be bound by theory, this relatively better stability may be due to a lower rate of dimerization. It is thought that the improved stability enables more reproducible measurements for the lactose-sensing assay on the optoelectronic device.

Calibration curves were performed using the aforementioned protocol by adding serial dilutions of lactose to PBS buffer (FIG. 31B). The wild-type sensor exhibited the highest lactose detection sensitivity (LOD: 0.0000527%, EC50: 0.00032), followed by LacB13 (LOD: 0.00063%, EC50: 0.0074%) and lastly, LacB6 and LacB16 (LOD: 0.0053%, EC50 0.0202% and LOD: 0.0027%, EC50: 0.0208%). Previous reports (Briggs et al., 2023; Caron & Trowell, 2018) showed that the wild-type sensing capacity was saturated in assays containing 50% (v/v) milk, due to the presence of trace levels of lactose and interference from high levels of glucose and galactose. The reduced sensitivity of LacB13 is beneficial for the lactose assay as it permits fewer milk dilution steps and simplifies the assay workflow. The remaining assay development was performed using the LacB13 biosensor.

The LacB13 biosensor was freeze dried as described in Example 3 using the following formulation:
250 mM Tris (pH7.5)
250 mM NaCl
12.5 mg/mL pullulan
12.5 mg/mL trehalose
0.45% (w/v) fish skin gelatine (*The stock solution contains 45% (w/v) (0.45 g/mL) fish skin gelatine.
250 nM LacB13 biosensor
50 U/mL porcine esterase

The formulation included 50 U/mL porcine esterase to achieve faster Compound-10 activation, thereby enabling shorter assay times. The freeze-dried reagent was reconstituted in 1 mL 10% lactose-free milk (Aldi Liddels) diluted in water, supplemented with one drop of C-10 (28 μL, 4.3 mM in dry DMSO) and analysed on the optoelectronic device for 4 min. The device was set to 37° C.

The optoelectronic device detected the bioluminescence generated by the sensor molecule (FIG. 31C). Rising BRET ratios were observed for the first 60 seconds followed by declining ratios (FIG. 31D) over the subsequent 3 minutes. To determine conditions for optimal stability of the BRET ratio within a 4 min assay time frame, we tested a range of various freeze-dried formulations (Table 10), where the concentrations of NaCl, Pullulan, trehalose and biosensor were varied.

TABLE 10
Lactose biosensor freeze-drying formulations

| | Tris | | | | | Lactose | | |
|---|---|---|---|---|---|---|---|---|
| | (pH | | | | Fish Skin | biosensor | | Porcine |
| Formulation | 7.5) | NaCl | Pullulan | Trehalose | Gelatine* | (LacB13) | | esterase |
| FD-L3-5 | 250 | 250 | 12.5 | 12.5 | 0.45% | 250 | nM | 50 U/mL |
| | mM | mM | mg/mL | mg/mL | (w/v) | | | |
| FD-L3-6 | 250 | 100 | 12.5 | 12.5 | 0.45% | 250 | nM | 50 U/mL |
| | mM | mM | mg/mL | mg/mL | (w/v) | | | |
| FD-L3-7 | 250 | 250 | 12.5 | 12.5 | 0.45% | 100 | nM | 50 U/mL |
| | mM | mM | mg/mL | mg/mL | (w/v) | | | |
| FD-L3-8 | 250 | 100 | 12.5 | 12.5 | 0.45% | 100 | nM | 50 U/mL |
| | mM | mM | mg/mL | mg/mL | (w/v) | | | |
| FD-L3-9 | 250 | 100 | 25 | — | 0.45% | 100 | nM | 50 U/mL |
| | mM | mM | mg/mL | | (w/v) | | | |
| FD-L3-10 | 250 | 100 | 25 | — | 0.45% | 50 | nM | 50 U/mL |
| | mM | mM | mg/mL | | (w/v) | | | |
| FD-L3-11 | 250 | 50 | 25 | — | 0.45% | 50 | nM | 50 U/mL |
| | mM | mM | mg/mL | | (w/v) | | | |
| FD-L3-12 | 250 | 100 | 25 | — | 0.45% | 25 | nM | 50 U/mL |
| | mM | mM | mg/mL | | (w/v) | | | |
| FD-L3-13 | 250 | 50 | 25 | — | 0.45% | 25 | nM | 50 U/mL |
| | mM | mM | mg/mL | | (w/v) | | | |

*The stock solution contains 45% (w/v) (0.45 g/mL) fish skin gelatine)

All tested formulations resulted in functional lactose sensors that could be used to analyse milk samples on the optoelectronic device. BRET ratios did not stabilise within 4 min for any of the tested formulations (FIG. 31E). Such stabilisation would be beneficial but is not required for assay utility. When lactose-free milk samples supplemented with 5% (w/v) lactose were analysed, the BRET ratios increased by up to 20% for trehalose-containing formulations, while ratios decreased up to 20% for formulations not containing trehalose (FIG. 31F). The disaccharide trehalose is added as a cryoprotectant. However, it is known to bind to the biosensor (Caron & Trowell, 2018), albeit much less strongly than lactose. We therefore suspected interference of trehalose with the lactose sensing capacity of the sensor as a drop in BRET ratio in the presence of lactose was expected. The highest dynamic range was seen for formulation FD-L3-13 (FIG. 31F), which does not contain any trehalose. While all formulations were capable of discriminating between lactose-free and lactose containing milk, only FD-L3-13 exhibited a stable BRET ratio differential comparing 1, 2, 3 or 4 min end-point measurements.

Next, formulation FD-L3-13 was used for further experimentation where serial dilutions of lactose were added to lactase-treated milk samples (Devondale Full Cream milk treated with 5000 NLU/L lactase for 6 days at 4° C. 1 mL aliquots were incubated at 95° C. for 10 mins to inactivate lactase and aliquots were stored at −80° C.). The lactose-sensing assay using freeze-dried reagents was able to discriminate between lactose-free milk and lactose-free milk supplemented with 5% (w/v) lactose (FIG. 31G) and showed a dose-dependent decrease in BRET ratio with increasing levels of lactose (FIG. 31H).

The method described here enables rapid and accurate determination of the lactose content of milk samples within 4 min on the optoelectronic device, using a shelf-stable two component formulation.

### Example 8. Detection of Plasmin Activity in Milk Samples Using Shelf-Stable Biosensor Reagents

Examples 1 to 5 demonstrate shelf-stable assay reagents that can be used to detect the activity of the protease AprX in milk samples on an optoelectronic device. The following example demonstrates that the methods and formulations illustrated in Examples 1 to 5 can be applied to detect other milk spoilage proteases, such as plasmin, in milk samples. Unlike AprX, it has been found that plasmin can be bound to its substrate casein. While detecting the activity directly in milk samples is possible, its detection sensitivity may be enhanced using a dissociation protocol (e.g. as described in WO2018/085895).

A plasmin BRET² sensor molecule (hereafter plasmin sensor) was constructed as described in WO2013/155553 (specifically Example 10 and FIG. 53 of that application, see also Dacres et al., (2019) A rapid and sensitive biosensor for measuring plasmin activity in milk, Sensors and Actuators B: Chemical, Volume 301, 127141) to generate the pRSET-Plasmin expression plasmid. The plasmin sensor was expressed and purified as described in the methods section. The purified plasmin sensor was then used to prepare two separate freeze-dried formulations as described below.

FD-Plasmin-5 Formulation: This formulation is used for the analysis of milk with an increased detection sensitivity for plasmin. It is intended to be used in conjugation conjunction with a sonication step, which homogenises milk and requires a higher esterase concentration. The formulation contains:
3,450 ng/ml plasmin BRET² biosensor
250 mM Tris (pH 8.4) buffer
250 mM NaCl
125 mM L-Lysine to support optimal plasmin activity
5 mM Aminocaproic acid for casein-plasmin dissociation
400 mM Trisodium acetic acid for casein-plasmin dissociation
0.45% (v/v) fish skin gelatine as carrier protein (*The stock solution contains 45% (w/v) (0.45 g/mL) fish skin gelatine)
12.5 mg/ml trehalose as cryoprotectant
12.5 mg/ml pullulan as cryoprotectant
25 U/ml porcine esterase as uncaging agent.

FD-Plasmin-6 Formulation: This formulation is for the analysis of unhomogenised milk. This formulation contains:
3,450 ng/ml plasmin BRET² biosensor
250 mM Tris (pH 8.4) buffer
250 mM NaCl
125 mM L-Lysine to support optimal plasmin activity
0.45% (v/v) fish skin gelatine as carrier protein (*The stock solution contains 45% (w/v) (0.45 g/mL) fish skin gelatine)
12.5 mg/ml trehalose as cryoprotectant
12.5 mg/ml pullulan as cryoprotectant
5 U/ml porcine esterase as uncaging agent.

The formulations were prepared in assay tubes and were freeze-dried as described in Example 3.

The freeze-dried FD-Plasmin-6 reagents were used in the protease assay described in Example 4. Briefly, The freeze-dried formulation was reconstituted with 1 mL raw milk supplemented with known amounts of standardised human plasmin protease were, and followed by the addition of 28 μL compound 10 solution (in dry DMSO, 4.3 mM) to provide an assay concentration of 120 μM compound 10. The tube was closed and inverted 20 times and inserted into the optoelectronic device. The activity of the human plasmin was standardised as described in the materials and methods section hereinabove.

Protease assays were performed using the optoelectronic device incubating at 37° C. and the BRET² bioluminescence signals were recorded over 600 sec (10 min) or 900 sec (15 min). Bioluminescence values from 590-600 sec were used to determine BRET ratio values for the 10 min assay and bioluminescence signal values recorded from 890-900 sec were used to determine BRET ratio values for the 15 min assay.

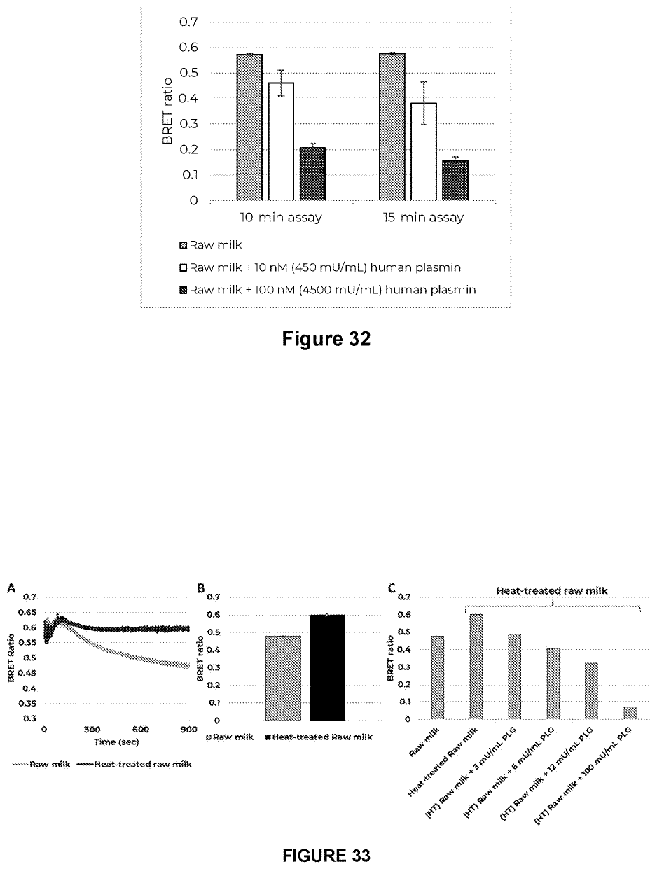

The observed BRET ratios decreased with increasing amounts of plasmin activity in the raw milk samples (FIG. 32). In 10-min assays the BRET ratio dropped from 0.572±0.004 to 0.461±0.050 in presence of 10 nM human plasmin (450 mU/mL) and to 0.206±0.018 in presence of 100 nM human plasmin (4500 mU/mL), respectively. In the 15-min assay BRET ratios dropped from 0.576±0.005 to 0.382±0.083 in presence of 10 nM human plasmin (450 mU/mL) and to 0.157±0.015 in presence of 100 nM human plasmin (4500 mU/mL). The bigger drop in BRET ratio suggests that the 15-min assay was more sensitive compared to a 10-min assay.

### Example 9. Detection of Plasminogen Activity in Milk Samples Using Shelf-Stable Biosensor Reagents

In milk or blood, plasmin is subject to a complex regulatory system, which includes an inactive precursor, plasminogen, and a cascade of plasminogen activators and inhibitors. Plasminogen present in milk samples can be activated to plasmin by temperature treatments that inadvertently change the balance of inhibitors and activators. High initial levels of plasminogen and/or heat-treatment processing faults, activating plasminogen to plasmin may lead to spoilage during long-term storage of long-life or UHT milk. In order to test whether the here-developed protease-sensing system can measure the latent plasmin-activity present as plasminogen in milk, we added the plasminogen activator urokinase to the assay reagent formulation developed in example 8.

The assay reagent formulation (referred to as FD-Plasminogen-1) was prepared as follows:
3,450 ng/ml plasmin BRET² biosensor
250 mM Tris (pH 8.4) buffer
250 mM NaCl
125 mM L-Lysine to support optimal plasmin activity
0.45% (v/v) fish skin gelatine as carrier protein (*The stock solution contains 45% (w/v) (0.45 g/mL) fish skin gelatine)
12.5 mg/ml trehalose as cryoprotectant
12.5 mg/ml pullulan as cryoprotectant
1000 U/mL human urokinase to activate bovine plasminogen
5 U/ml porcine esterase as uncaging agent.

The FD-Plasminogen-1 was freeze-dried (FD) as described in Example 3 and the protease assay protocol developed in Example 4 was used for the analysis of plasminogen in milk samples.

Naturally present plasminogen in raw milk was deactivated by incubating 1 mL sample in a 1.5 mL Eppendorf tube at 90° C. for 20 min. The freeze-dried formulation was reconstituted with 1 mL raw milk (either heat treated or unheated raw milk). This was followed by the addition of 28 μL compound 10 solution (in dry DMSO, 4.3 mM) to provide an assay concentration of 120 UM compound 10. The tube was closed and inverted 20 times and inserted into the optoelectronic device.

Protease assays were performed using the optoelectronic device incubating at 37° C. and the BRET² bioluminescence signals were recorded over 900 sec (15 min). Bioluminescence values from 890-900 sec were used to determine BRET ratio values.

Following initial changes (<100 seconds) in BRET ratio due to generation of free coelenterazine in the system, unheated raw milk samples exhibited a continuous decrease in BRET ratio, while the BRET ratios of heat-treated raw milk samples stabilised (FIG. 33A). This indicates the presence of bovine plasminogen that was activated by human urokinase producing plasmin. The plasmin protease was then detectable by the plasmin sensor. Using a 15-min assay, BRET ratios revealed a 20% decrease in BRET ratio comparing raw milk and heat-treated raw milk samples (FIG. 33B). The protease assay was then run using heat-treated raw milk samples spiked with serial dilutions of human plasminogen. A dose-dependent response with higher levels of plasminogen resulting in lower BRET ratios was observed (FIG. 33C). This example demonstrates that the methods and formulation described herein can be used to measure latent activity levels of bovine plasminogen in raw milk samples.

### Example 10. Detection of Subtilisin (Alcalase) Activity in Milk Samples Using Shelf-Stable Biosensor Reagents and Discrimination Against AprX Activity

Spores of the bacterial spore-forming genus Bacillus occur in milk powder and, following hydration and spore germination, secrete several different proteases in milk. The main protease class, the subtilases (also known as subtilisin or alcalase), are serine proteases that preferentially cleave uncharged amino acids. We hypothesised that the CT-AprX biosensor, which includes neutral amino acids, may be cleaved by subtilases present in reconstituted milk powder with the required sensitivity to be of practical use to the dairy industry.

Freeze-dried AprX biosensor formulations developed in Example 3 were prepared with a 25 U/mL (final working concentration 5 u/mL????) porcine esterase concentration and the BRET² protocol developed in example 4 was used to analyse UHT milk samples spiked with activity-calibrated subtilisin. Subtilisin was activity calibrated following the AprX protease activity calibration protocol described in the methods section.

Protease assays were performed using the optoelectronic device incubating at 37° C. and the BRET² bioluminescence signals were recorded over 600 sec (10 min) or 900 sec (15 min). Bioluminescence values from 590-600 sec were used to determine BRET ratio values for a 10 min assay and bioluminescence signal values recorded from 890-900 sec were used to determine BRET ratio values for a 15 min assay.

In order to discriminate between AprX and subtilisin activity, phenylmethylsulfonyl fluoride (PMSF), a serine-protease inhibitor, was added to milk samples spiked with AprX or subtilisin at a final concentration of 0.8 mM. Following addition of PMSF, the samples were incubated on ice for 30 min.

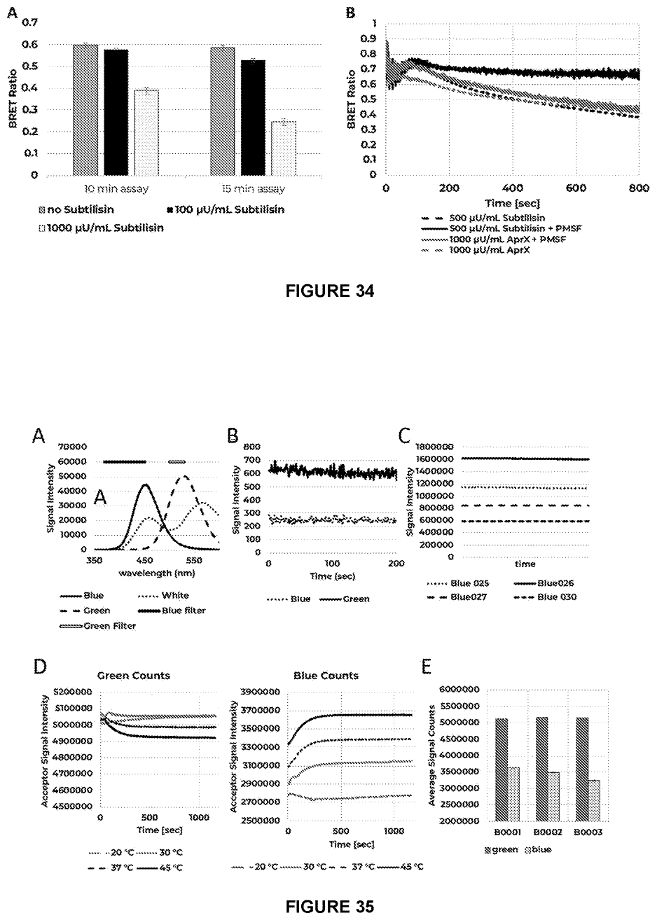

Analysed UHT milk samples exhibited a subtilisin activity dependent decrease in BRET ratios for both 10-min and 15-min assays (FIG. 34A). The 15-min assay exhibited a larger drop in BRET ratios compared to the 10-min assay. BRET ratios decreased over time when milk samples spiked with 500 μU/mL subtilisin or 1000 μU/mL AprX were analysed (FIG. 34B). Samples spiked with 1000 μU/mL AprX and treated with PMSF exhibited a similar drop in BRET ratios over time, showing that AprX was not inhibited by PMSF. In contrast, PMSF treated subtilisin-spiked milk samples led to stable BRET ratios (FIG. 34B).

### Example 11. Calibration Method for Light Detectors in Optoelectronic Device Using a Gaseous Tritium Light Source

The optoelectronic device described herein contains two micro photon multiplier tubes (μPMTs). The photon detection sensitivity of those μPMTs differs between devices and individual μPMT performance will decay over time. To ensure accurate and precise measurements, the PMT signal output should be calibrated and adjusted over time.

To accomplish this, we tested whether gaseous tritium light sources (GTLS) can be used to calibrate the optoelectronic device. Various Trigalight® GTLS sources (all <0.67 GBq, non-radiative) were purchased from SP2 (Self Powered Safety Products Ltd, United Kingdom) and their luminescence properties were tested in response to relevant external factors (temperature and light exposure.

According to the supplier's spec sheet:
“Each MiniGlow Safety Marker incorporates a Trigalight® Gaseous Tritium Light Source (GTLS) which provides a reliable, safe and self-powered source of continuous light output. No battery or charging by ambient light is required during the lifetime of the product. GTLS comprise a sealed borosilicate glass capsule which contains a tiny amount of gaseous tritium, the power supply for the device. Beta-particle radiation (i.e. electrons released by the gaseous tritium as it decays) is harnessed to energise a phosphor coating on the inside surface of the capsule, causing it to glow. As a sealed source, they are non-emissive and of a very low energy level. Every Trigalight® source light is manufactured and tested in accordance with the UK MOD Defence Standard 62/4. GTLS contain no moving parts and, because the unit is continuously energised, an off/on switch is not required. They will remain energised for more than 10 maintenance-free years, even during extended periods of complete darkness. Trigalight® devices can be used in any environmental conditions (including under-water applications) and in temperatures ranging from −40° to 80° C. They are suitable for operation in Zone 0 hazardous areas . . . .
Specification
MiniGlow Markers are moulded in clear grade V0 polycarbonate with stainless steel end-caps and split-ring [that is removed], providing a tough protective enclosure and making them highly shock resistant. Length: 18.0 mm Diameter: 9.0 mm Overall Weight: 3.0 g Clip Diameter: 20.0 mm Maximum Activity: 0.67 GBq

First, in order to select the ideal ‘colour’ for the filter sets installed in the optoelectronic device, we analysed the luminescence spectra of three different colours (UV blue, green and white) of GTLS tubes provided as cylindrically shaped tubes moulded into a hexagonally-shaped transparent polycarbonate casing (“Micro Markers” UV Blue, Green and White). “Luminescence spectra were determined on a CLARIOstar plate reader by placing GTLS sources into a 96-well plate followed by measuring the luminescence from 320-610 nm (gain: 2000 at 30° C.).

Spectral scans showed that photons from the ‘blue’ and ‘white’ GTLS sources would pass through both the green and blue optical filter windows of optoelectronic device, while the ‘green’ source exhibited little luminescence within the ‘blue filter’ window (FIG. 35A). The ‘white’ GTLS source showed two emission peaks, which was considered an unnecessary complication. Therefore, the GTLS with the “UV blue” phosphor was selected for all future experiments

We tested the positioning of the GTLS source within the optoelectronic device tube holder by either temporarily adhering the GTLS source to the screw cap of a 2 mL tube using “Blu Tack” adhesive (FIG. 35B) or by placing the GTLS source at the bottom of a capped 2 mL tube (FIG. 35C). The device temperature was set to 37° C. and luminescent counts of both channels were recorded over 5 min.

The cap-mounted GTLS led to low amounts of light (FIG. 35B; <1000 counts) reaching the PMTs, but GTLS tubes placed at the bottom of the tube holder gave signal counts of 500,000-6,000,000, depending on the type of the GTLS and version of the Device hardware (FIG. 35C). The counts for the latter experiment also showed a high signal variation when the tube containing the GTLS source was repeatably removed and reinserted into the tube holder, likely due a non-homogenous spread of GTLS light emission. This showed that the GTLS source had to be placed at the bottom or within the bottom half of the tube holder and at a fixed angle relative to the detection windows of the μPMTs.

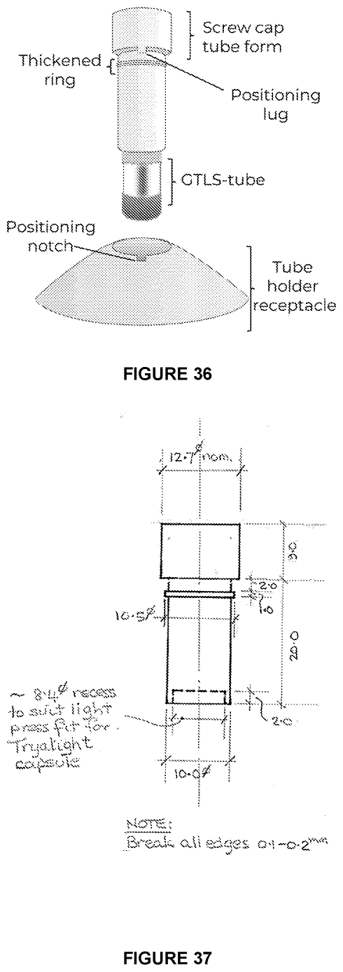

We then purchased and tested two different types of Blue/UV Blue Trigalight® GTLS (“Bivvy Marker” (TD302) or “MiniGlow Smooth” (TD912). The blue colour of these was the closest available match to the “UV Blue” used in the initial experiments. These two types have the benefit of the GTLS source being encased in an approximately cylindrical (rather than a polygonal prism) polycarbonate holder. For ease of fabrication, we selected only the MiniGlow Smooth because it has flat metal caps on both ends that facilitate attachment to the holder. We glued the top end of the Mini Glow Smooth GTLS (which has a transverse hole for attachment to a keyring) to a recessed socket incorporated into the base of a lathe-turned facsimile of the upper half of a 2 mL screw cap tube (Thermo Scientific™ QSP™) (FIG. 36 and FIG. 37). Additionally, the calibration tool was provided with a thickened region, 2 mm below the cap, to ensure a “transition” or push fit of the trigalight in the tube holder of the device, stabilising the tube within the holder.

In a further refinement, and to ensure precisely repeatable rotational alignment of the calibration tool in the device, we machined a positioning notch into the top of the tube-holder of the optoelectronic device and we designed a complementary lug to fit into said notch for the cap of the calibration tool. In order to demonstrate the utility of such an approach (prior to delivery of the handles with lug), we used an engraved and marked dot to visually align the calibration tube with the notch. When assembling and gluing the GTLS into the holder, we used a jig that allows the injection moulding seam of the polycarbonate casing to be aligned with the axis of the lug/dot, thereby ensuring the seam is at right angles to the axis of the PMTs in the device and minimising variance in light transmission to the PMTs due to any inhomogeneity in the optical properties of the polycarbonate at or near the seam.

The calibration source (referred to as CT2) was then tested on the optoelectronic device by monitoring ‘blue’ and ‘green’ channel luminescence over 20 min at 37° C.

‘Green’ channel counts increased over time by ~9% until reaching a stable plateau signal level, while ‘blue’ channel counts decreased by ~0.1% before reaching a stable plateau. The same effect albeit at different plateau levels was observed when CT2 was analysed at other device temperatures (20-45° C.) (FIG. 35D). We speculate that the temperature of the GTLS source itself affects luminescent output, likely due to changes of the efficiency with which Tritium electrons excite the phosphor coating on the inside of the tubes. For precise measurements, therefore, CT2 should be equilibrated to a set device temperature before using signals for calibration purposes.

CT2 was tested repeatedly by removing and reinserting the tube after each finished assay. Three optoelectronic devices were tested. Each assay was analysed for 5 min at 37° C. and photon counts between 200-300 sec were averaged. The first run was not included in the data analysis as it was used to equilibrate CT2 to the device temperature.

CT2 signals were highly reproducible (FIG. 35E) with a coefficient of variation ranging from 0.01 to 0.17% for the ‘green’ channels and 0.13-0.56% of the ‘blue’ channels, comparing all three devices.

Setting a reference datum based on those measurements enabled us to adjust the signal output of newly manufactured optoelectronic devices. Additionally, correcting for the decline in luminance due to tritium decay (λ=12.33 years), reference data sets were calculated to adjust for the reduction in luminance of individual calibration tools over time, thereby isolating any change in photon sensitivity of μPMTs that may occur over time.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present application claims priority from AU2022903116 filed 21 Oct. 2022, the entire contents of which is incorporated herein by reference.

The present application is filed together with a Sequence Listing in electronic form. The entire contents of the Sequence Listing are hereby incorporated by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Future patent applications may be filed in Australia or overseas on the basis of or claiming priority from the present application. It is to be understood that the following provisional claims are provided by way of example only, and are not intended to limit the scope of what may be claimed in any such future application. Features may be added to or omitted from the provisional claims at a later date so as to further define or re-define the invention or inventions.

1. A method of detecting an extracellular analyte in a sample, comprising:
providing a biosensor system selected from (i) an extracellular bioluminescent sensor molecule comprising a donor lumiphore, an analyte-sensitive domain and an acceptor fluorophore; and (ii) a precursor system thereof, the precursor system comprising a pair of molecules adapted to react in solution to form the extracellular bioluminescent sensor molecule;
incubating an assay composition comprising the sample and the biosensor system for an incubation period sufficient for the extracellular analyte, if present in the sample, to interact with the analyte-sensitive domain; and
detecting light emitted from the assay composition after the incubation period, wherein the ratio of luminosity associated with the acceptor fluorophore to luminosity associated with the donor lumiphore indicates an interaction of the extracellular analyte with the analyte-sensitive domain,
wherein at least one caged substrate compound is uncaged in the assay composition during the incubation period to produce an uncaged substrate compound which energises the donor lumiphore for the detecting.

2. The method according to claim 1, wherein the at least one caged substrate compound is present in the assay composition from the beginning of the incubation period.

3. The method according to claim 1 or claim 2, wherein the at least one caged substrate compound is uncaged by an uncaging agent present in the assay composition, preferably from the beginning of the incubation period.

4. The method according to claim 3, wherein the uncaging agent is extraneous to the sample, preferably wherein the uncaging agent is an esterase extraneous to the sample.

5. The method according to claim 3, wherein the uncaging agent is endogenous to the sample, preferably wherein the uncaging agent is an esterase endogenous to the sample.

6. The method according to any one of claims 1 to 5, wherein the incubation period is between 1 minute and 30 minutes.

7. The method according to any one of claims 1 to 6, wherein the incubation period is between 5 and 15 minutes.

8. The method according to any one of claims 1 to 7, wherein the at least one caged substrate compound is uncaged to provide a luminosity associated with the acceptor fluorophore, in the absence of the extracellular analyte, greater than 80% of maximum when detecting the light.

9. The method according to claim 8, wherein the luminosity associated with the acceptor fluorophore, in the absence of the extracellular analyte, is greater than 80% of maximum throughout a detection window of at least 3 minutes, preferably at least 5 minutes, wherein the light is detected during the detection window.

10. The method according to any one of claims 1 to 9, wherein at least two different caged substrate compounds present in the assay composition from the beginning of the incubation period are uncaged during the incubation period, thereby producing the uncaged substrate compound at different reaction rates.

11. The method according to any one of claims 1 to 10, wherein each caged substrate compound is a caged coelenterazine compound comprising a carbonyl protecting group at the 3′-position.

12. The method according to any one of claims 1 to 11, wherein each caged substrate compound has a structure according to Formula 1 or Formula 2: 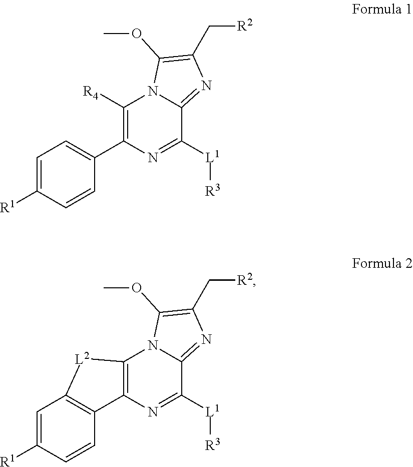
wherein:
each R¹ is independently selected from H, OH, OR^{alk} where R^{alk} is C₁-C₆ alkyl, and NH₂,
each R² is independently selected from H, Ar, and (CH₂)ₙAr, where each Ar is independently a carbocyclic aromatic group optionally substituted with OH, C₁-C₆ alkyl or halogen, and n is 1 or 2,
each L¹ is independently selected from CH₂ or S,
each R³ is independently selected from C₆H₅, CH₂C₆H₅, C₁-C₆ alkyl, and C₁-C₆ cycloalkyl,
R⁴ is independently selected from H and CH₃,
L² is independently selected from (CH₂)ₙ where n is 1-3 and CH═CH, and
each X is independently a carbonyl protecting group.

13. The method according to claim 12, where each X has a structure according to Formula 3, Formula 4 or Formula 5: 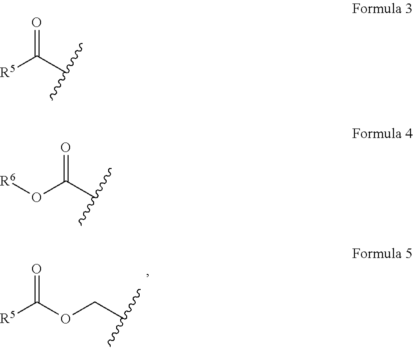
wherein R⁵, R⁶ and R⁷ are each independently optionally substituted C₁-C₁₂ alkyl, C₁-C₁₂ cycloalkyl or C₁-C₁₂ aryl.

14. The method according to any one of claims 1 to 13, wherein the uncaged substrate compound is a coelenterazine compound, preferably bisdeoxycoelenterazine.

15. The method according to any one of claims 1 to 14, wherein the at least one caged substrate compound is introduced to the assay composition in a room temperature-stable substrate solution, preferably by dispensing the substrate solution drop-wise into the assay composition.

16. The method according to claim 15, wherein the substrate solution is a non-aqueous solution comprising a water-miscible aprotic solvent, preferably DMSO.

17. The method according to claim 15 or claim 16, further comprising storing the substrate solution for at least one week, preferably at least one month, more preferably at least six months, before introducing the substrate solution to the assay composition.

18. The method according to any one of claims 1 to 17, comprising preparing the assay composition by a method comprising dispersing a solid composition comprising the biosensor system in a liquid, preferably wherein the solid composition is a freeze-dried solid composition and preferably wherein the liquid comprises the sample.

19. The method according to claim 18, wherein the solid composition further comprises an uncaging agent for uncaging the at least one caged substrate compound in the assay composition.

20. The method according to claim 18 or claim 19, wherein the assay composition is prepared and incubated in an assay vessel in which the solid composition was previously freeze-dried.

21. The method according to any one of claims 1 to 20, wherein the assay composition is prepared and incubated in an assay vessel and the light emitted from the assay composition is detected through the walls of the assay vessel.

22. The method according to any one of claims 1 to 21, wherein the extracellular bioluminescent sensor molecule is a BRET² biosensor.

23. The method according to any one of claims 1 to 22, wherein the extracellular analyte is selected from the group consisting of an enzyme, a zymogen and a carbohydrate, and is preferably a protease.

24. The method according to any one of claims 1 to 23, wherein the sample is a food or beverage composition, preferably a dairy composition.

25. The method according to any one of claims 1 to 24, comprising:
providing the sample, as a liquid, for analysis;
providing an assay vessel containing a solid composition, the solid composition comprising the extracellular bioluminescent sensor molecule and optionally an uncaging agent;
providing a substrate storage container containing a room temperature-storable substrate solution, the room temperature-storable substrate solution comprising the at least one caged substrate compound;
adding an aliquot of the sample to the assay vessel;
thereafter, transferring an aliquot of the room temperature-storable substrate solution from the substrate storage container into the assay vessel, and mixing the assay composition contained therein;
incubating the assay composition in the assay vessel for the incubation period; and
detecting the light emitted from the assay composition through the walls of the assay vessel with an optoelectronic device.

26. A system for detecting an extracellular analyte in a sample, comprising:
a biosensor system selected from (i) an extracellular bioluminescent sensor molecule comprising a donor lumiphore, an analyte-sensitive domain and an acceptor fluorophore; and (ii) a precursor system thereof, the precursor system comprising a pair of molecules adapted to react in solution to form the extracellular bioluminescent sensor molecule, the biosensor system contained in an assay vessel configured to receive the sample for combination with the biosensor system in an assay composition;
a room temperature-stable substrate solution comprising at least one caged substrate compound, for dispensing into the assay composition in the assay vessel, the substrate solution contained in a substrate storage vessel; and
an optoelectronic device configured to detect light emitted from the assay composition through the walls of the assay vessel, wherein the ratio of luminosity associated with the acceptor fluorophore to luminosity associated with the donor lumiphore indicates an interaction of the extracellular analyte with the analyte-sensitive domain,
wherein the at least one caged substrate compound is adapted to uncage in the assay composition, thereby producing an uncaged substrate compound which energises the donor lumiphore.

27. The system according to claim 26, wherein the optoelectronic device is configured to receive the assay vessel and to incubate the assay composition contained therein for an incubation period sufficient for the extracellular analyte, if present in the sample, to interact with the analyte-sensitive domain, preferably wherein the optoelectronic device is configured to incubate the assay composition at a predetermined incubation temperature.

28. The system according to claim 26 or claim 27, wherein the biosensor system is present in a solid composition, preferably a freeze-dried solid composition.

29. The system according to claim 28, wherein the solid composition further comprises an uncaging agent for uncaging the at least one caged substrate compound in the assay composition.

30. The system according to any one of claims 26 to 29, wherein the substrate solution comprises at least two different caged substrate compounds adapted to uncage in the assay composition to produce the uncaged substrate compound at different reaction rates.

31. The system according to any one of claims 26 to 30, wherein each caged substrate compound is a caged coelenterazine compound comprising a carbonyl protecting group at the 3′-position.

32. The system according to any one of claims 26 to 31, wherein each caged substrate compound has a structure according to Formula 1 or Formula 2: 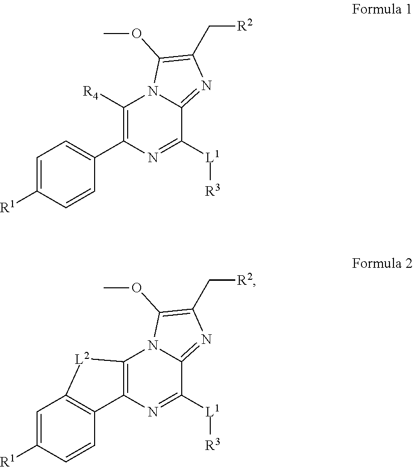
wherein:
each R¹ is independently selected from H, OH, OR^{alk} where R^{alk} is C₁-C₆ alkyl, and NH₂,
each R² is independently selected from H, Ar, and (CH₂)ₙAr, where each Ar is independently a carbocyclic aromatic group optionally substituted with OH, C₁-C₆ alkyl or halogen, and n is 1 or 2,
each L¹ is independently selected from CH₂ or S,
each R³ is independently selected from C₆H₅, CH₂C₆H₅, C₁-C₆ alkyl, and C₁-C₆ cycloalkyl,
R⁴ is independently selected from H and CH₃,
L² is independently selected from (CH₂)ₙ where n is 1-3 and CH═CH, and
each X is independently a carbonyl protecting group.

33. The system according to claim 32, where each X has a structure according to Formula 3, Formula 4 or Formula 5: 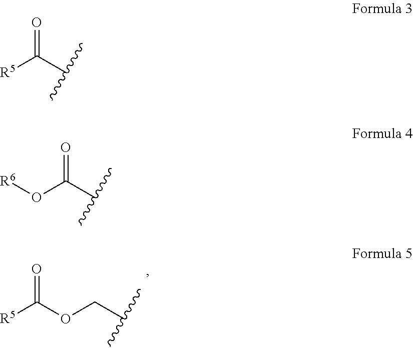
wherein R⁵, R⁶ and R⁷ are each independently optionally substituted C₁-C₁₂ alkyl, C₁-C₁₂ cycloalkyl or C₁-C₁₂ aryl.

34. The system according to any one of claims 26 to 33, wherein the uncaged substrate compound is a coelenterazine compound, preferably bisdeoxycoelenterazine.

35. The system according to any one of claims 26 to 34, wherein the substrate solution is a non-aqueous solution comprising a water-miscible aprotic solvent, preferably DMSO.

36. The system according to any one of claims 26 to 35, wherein the substrate storage vessel is configured to dispense an aliquot of the substrate solution into the assay vessel, and is preferably configured to dispense the substrate solution drop-wise into the assay vessel.

37. The system according to any one of claims 26 to 36, wherein the extracellular bioluminescent sensor molecule is a BRET² biosensor.

38. The system according to any one of claims 26 to 37, wherein the optoelectronic device comprises a first light detector comprising a micro-photon multiplier tube fitted with a band pass filter to selectively permit transmission of light wavelengths associated with the acceptor fluorophore and a second light detector comprising a micro-photon multiplier tube fitted with a band pass filter to selectively permit transmission of light wavelengths associated with the donor lumiphore.

39. The system according to any one of claims 26 to 38, wherein the optoelectronic device is configured to incubate the assay composition at a predetermined incubation temperature.

40. A BRET reagent system for combination with a sample to produce a bioluminescent assay composition having a BRET ratio, after incubation of the assay composition, that is indicative of an extracellular analyte in the sample, the BRET reagent system comprising:
(a) a solid composition comprising a biosensor system selected from (i) an extracellular bioluminescent sensor molecule comprising a donor lumiphore, an analyte-sensitive domain susceptible to interaction with the extracellular analyte, and an acceptor fluorophore; and (ii) a precursor system thereof, the precursor system comprising a pair of molecules adapted to react in solution to form the extracellular bioluminescent sensor molecule, wherein the solid composition is dispersible in a liquid comprising the sample to produce the assay composition; and
(b) a room temperature-stable substrate solution comprising at least one caged substrate compound, for dispensing into the assay composition,
wherein the at least one caged substrate compound is adapted to uncage in in the assay composition to produce an uncaged substrate compound which energises the donor lumiphore.

41. The BRET reagent system according to claim 40, wherein the solid composition is contained in an assay vessel configured to receive the liquid comprising the sample and to permit transmission of light from the assay composition through the assay vessel walls for detection.

42. The BRET reagent system according to claim 41, wherein the solid composition is a freeze-dried composition prepared by freeze-drying an aqueous precursor composition in the assay vessel.

43. The BRET reagent system according to any one of claims 40 to 42, wherein the solid composition further comprises an uncaging agent for uncaging the at least one caged substrate compound in the assay composition.

44. The BRET reagent system according to any one of claims 40 to 43, wherein the room temperature-stable substrate solution is contained in a substrate storage vessel, the substrate storage vessel configured to dispense an aliquot of the substrate solution into the assay composition, and preferably configured to dispense the substrate solution drop-wise into the assay composition.

45. The BRET reagent system according to any one of claims 40 to 44, wherein the room temperature-stable substrate solution comprises at least two different caged substrate compounds adapted to uncage in the assay composition to produce the uncaged substrate compound at different reaction rates.

46. The BRET reagent system according to any one of claims 40 to 45, in the form of a kit comprising instructions for use of the BRET reagent system in a method according to any one of claims 1 to 25.

47. A solid biosensor composition for BRET detection of an extracellular analyte in a sample, the solid biosensor composition comprising:
(a) a biosensor system selected from (i) an extracellular bioluminescent sensor molecule comprising a donor lumiphore, an analyte-sensitive domain susceptible to interaction with the extracellular analyte, and an acceptor fluorophore; and (ii) a precursor system thereof, the precursor system comprising a pair of molecules adapted to react in solution to form the extracellular bioluminescent sensor molecule; and
(b) an uncaging agent,
wherein the solid biosensor composition is dispersible in a liquid comprising the sample to produce an assay composition, and
wherein the uncaging agent is adapted to uncage at least one caged substrate compound in the assay composition, thereby producing an uncaged substrate compound which energises the donor lumiphore.

48. A substrate dispensing system for BRET detection of an extracellular analyte in a sample, the substrate dispensing system comprising a room temperature-stable substrate solution comprising at least one caged substrate compound, wherein the substrate solution is contained in a substrate storage vessel configured to dispense a pre-determined aliquot of the substrate solution into an assay composition comprising the sample and a BRET bioluminescent sensor molecule.

49. The substrate dispensing system according to claim 48, wherein the room temperature-stable substrate solution comprises at least two different caged substrate compounds adapted to uncage at different reaction rates.

50. A substrate composition for BRET detection of an extracellular analyte in a sample, the substrate composition comprising at least two different caged substrate compounds adapted to uncage in an assay composition comprising the sample and a BRET bioluminescent sensor molecule, thereby producing an uncaged substrate compound at different reaction rates.
